(19) United States
(12) Patent Application Publication
(10) Pub. No.: US 2026/0365661 A1
(43) Pub. Date: Nov. 19, 2026

(54) METHODS OF MANUFACTURING KINASE INHIBITORS

(71) Applicant: Insilico Medicine IP Limited, Hong Kong (CN)

(72) Inventors: Xing LIANG, Shanghai (CN); Hua CAO, Shanghai (CN); Xin CHENG, Shanghai (CN); Luoheng QIN, Shanghai (CN)

(21) Appl. No.: 19/103,784

(22) PCT Filed: Aug. 23, 2023
(86) PCT No.: PCT/CN2023/114387

§ 371 Date: Feb. 13, 2025

(30) Foreign Application Priority Data
Aug. 24, 2022 (WO) PCT/CN2022/114447

(51) Int. Cl. C07D 487/10; C07D 401/04; C07D 403/04; C07D 403/14; C07D 405/04

(52) U.S. Cl. CPC C07D 487/10; C07D 401/04; C07D 403/04; C07D 403/14; C07D 405/04

(57) ABSTRACT

In one aspect, the disclosure provides methods of synthesizing TNIK and/or MAP4K4 kinases inhibitors for the treatment of disease. In one aspect, the disclosure provides intermediates used in the synthesis methods described herein.

### CROSS REFERENCE TO RELATED APPLICATIONS

This international patent application claims the benefit of International Application No. PCT/CN2022/114447, filed Aug. 24, 2022, which is incorporated herein by reference in its entirety.

### BACKGROUND

A biologically active enzyme known as Traf2- and Nck-interacting protein kinase is an enzyme commonly known as the TNIK in humans, and which is encoded by the TNIK gene. TNIK as a serine/threonine kinase is involved in various biological processes. There is a need for new drug candidates that can target TNIK.

### SUMMARY

In one aspect, described herein are methods of making kinase inhibitors (e.g., TNIK inhibitors) and intermediates used in the methods. TNIK is a serine/threonine kinase that is involved in various biological processes including acting as an essential regulatory component of the Wnt signaling pathway. TNIK can directly bind TCF4 and b-catenin and phosphorylates TCF4. Additionally, TNIK can play an activator of Wnt target gene expression and modulates the actin cytoskeleton and activates the c-Jun N-terminal kinase pathway, which is responsive to stress. It is also part of a signaling complex composed of NEDD4, RAP2A, and TNIK, which regulates neuronal dendrite extension and arbonization during development. More generally, TNIK may play a role in cytoskeletal rearrangement and regulate cell spreading. TNIK also causes weak Smad1 T322 phosphorylation, involved in TGF-b1 signaling transduction.

TNIK is considered to be a germinal center kinase (GCK), which can be characterized by an N-terminal kinase domain and a C-terminal GCK domain that serves a regulatory function.

TNIK activation of Wnt signaling can play important roles in carcinogenesis and embryonic development. Mutations in this gene are associated with an autosomal recessive form of cognitive disability.

Additionally, TNIK is linked to cancer, including for example, colorectal cancer. As such, TNIK has been identified as an attractive candidate for drugs targeting certain cancers.

The current data may imply TNIK is a potential target for the generation of small molecule inhibitors to specifically block the Wnt pathway in disease states such as colorectal cancer or the autosomal recessive form of cognitive disability.

Also, therapeutic targets associated with EMT, such as TNIK being target for inhibition, can be used for therapies for treating and/or preventing EMT-based disorders, such as cancer metastasis and fibrosis.

Accordingly, it would be advantageous to have a TNIK inhibitor that can inhibit the kinase activity of TNIK, as a member of the Ste20 family of MAP kinase kinase kinase (MAP4K).

In one aspect, provided herein is a method of producing a compound represented by Formula (I), or a pharmaceutically acceptable salt thereof,

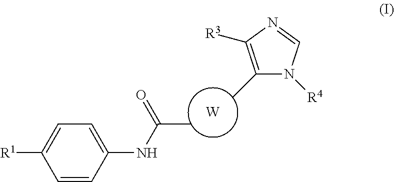

wherein:
R¹ is selected from:
   N(R⁵)₂, wherein each R⁵ is independently selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle; and
   optionally substituted 3 to 8-membered heterocycle; wherein the 3 to 8-membered heterocycle is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, and —NO₂;
R³ is selected from optionally substituted C₁-C₆ alkyl, optionally substituted 3- to 10-membered heterocycle and optionally substituted C₃₋₁₀ carbocycle, wherein each of the alkyl, heterocycle and carbocycle is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R⁴ is selected from:
   hydrogen;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
   optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
Ring W is an optionally substituted 5 or 6-membered heteroaryl, wherein the 5 or 6-membered heteroaryl is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle,
wherein the method comprises subjecting a compound represented by Formula (A), or a pharmaceutically acceptable salt thereof,

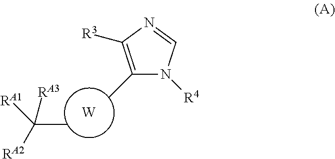

wherein Ring W, R³ and R⁴ are defined above, and
each of R^{A1}, R^{A2} and R^{A3} is independently selected from a halogen and —O—C₁₋₆alkyl;
through a hydrolysis step to produce a compound represented by Formula (B), or a pharmaceutically acceptable salt thereof,

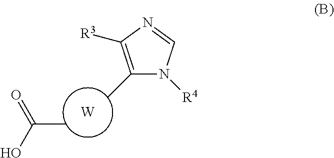

wherein Ring W, R³ and R⁴ are defined above.

In one aspect, provided herein is a method of producing a compound represented by Formula (I), or a pharmaceutically acceptable salt thereof,

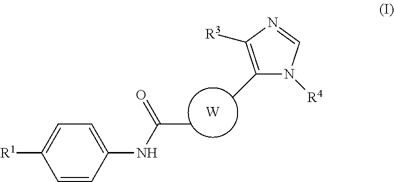

wherein:
R¹ is selected from:
   N(R⁵)₂, wherein each R⁵ is independently selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₁₀ alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle; and
   optionally substituted 3 to 8-membered heterocycle; wherein the 3 to 8-membered heterocycle is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, and —NO₂;
R³ is selected from optionally substituted C₁-C₆ alkyl, optionally substituted 3- to 10-membered heterocycle and optionally substituted C₃₋₁₀ carbocycle, wherein each of the alkyl, heterocycle and carbocycle is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₂₀ carbocycle, and 3- to 12-membered heterocycle;
R⁴ is selected from:
   hydrogen;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
   optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
Ring W is an optionally substituted 5 or 6-membered heteroaryl, wherein the 5 or 6-membered heteroaryl is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle,
wherein the method comprises reacting a compound represented by Formula (E), or a pharmaceutically acceptable salt thereof,

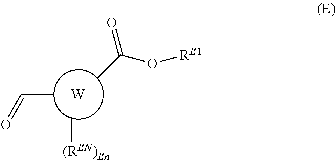

wherein,
   Ring W is defined above,
   En is 0 or 1,
   R^{EN} is a protecting group, and
   R^{E1} is C₁-C₆ alkyl (e.g., ethyl), C₁-C₆haloalkyl, C₁-C₆hydroxyalkyl, C₁-C₆aminoalkyl, C₁-C₆heteroalkyl, substituted or unsubstituted aryl, or substituted or unsubstituted heteroaryl;
with a compound represented by Formula (F), or a pharmaceutically acceptable salt thereof,

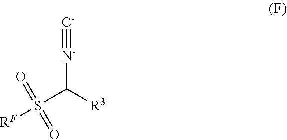

wherein R³ is defined above, and R^{F} is a substituted or unsubstituted phenyl,
in the presence of a compound of Formula (K), or a pharmaceutically acceptable salt thereof,

R⁴—NH₂ (K),
thereby producing a compound represented by Formula (G), or a pharmaceutically acceptable salt thereof,

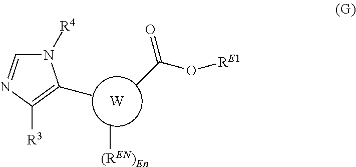

wherein R³, R⁴, Ring W, En, R^{EN} and R^{E1} are defined above.

In one aspect, provided herein is a method of producing a compound represented by Formula (I), or a pharmaceutically acceptable salt thereof,

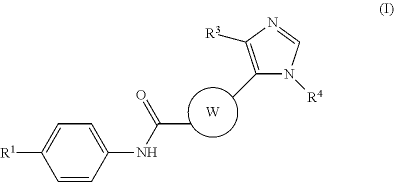

wherein:
R¹ is selected from:
   N(R₅)₂, wherein each R⁵ is independently selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₁₀ alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle; and optionally substituted 3 to 8-membered heterocycle; wherein the 3 to 8-membered heterocycle is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is
   optionally substituted with one or more substituents independently selected at each occurrence from hydroxy, halogen, oxo, —C₁₋₁₀haloalkyl, —NH₂, —CN, and —NO₂;
R³ is selected from optionally substituted C₁-C₆ alkyl, optionally substituted 3- to 10-membered heterocycle and optionally substituted C₃₋₁₀ carbocycle, wherein each of the alkyl, heterocycle and carbocycle is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
R⁴ is selected from:
   hydrogen;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
   optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
Ring W is an optionally substituted 5 or 6-membered heteroaryl, wherein the 5 or 6-membered heteroaryl is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle,
wherein the method comprises reacting a compound represented by Formula (E), or a pharmaceutically acceptable salt thereof,

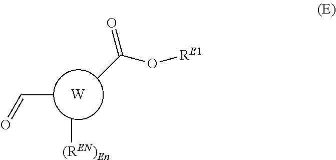

wherein,
   Ring W is defined above,
   En is 0,
   R^{EN} is a protecting group, and
   R^{E1} is C₁-C₆ alkyl (e.g., ethyl), C₁-C₆haloalkyl, C₁-C₆hydroxyalkyl, C₁-C₆aminoalkyl, C₁-C₆heteroalkyl, substituted or unsubstituted aryl, or substituted or unsubstituted heteroaryl;
with a compound represented by Formula (F), or a pharmaceutically acceptable salt thereof,

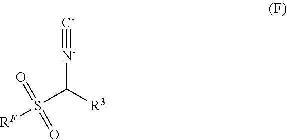

wherein R³ is defined above, and
R^{F} is a substituted or unsubstituted phenyl,
thereby producing a compound of Formula (G-a), or a pharmaceutically acceptable salt thereof,

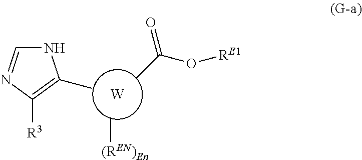

wherein R³, Ring W, En, R^{EN} and R^{E1} are defined above, and wherein the method further comprises reacting the compound of Formula (G-a), or a pharmaceutically acceptable salt thereof, with a compound of Formula (J), or a pharmaceutically acceptable salt thereof,

R⁴—R^{J} (J)

wherein R is a leaving group and R⁴ is defined above, thereby producing a compound represented by Formula (G), or a pharmaceutically acceptable salt thereof,

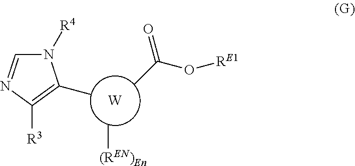

wherein R³, R⁴, R^{EN}, En, and R^{E1} are defined above.

In one aspect, provided herein is a method of producing a compound represented by Formula (Ia), or a pharmaceutically acceptable salt thereof,

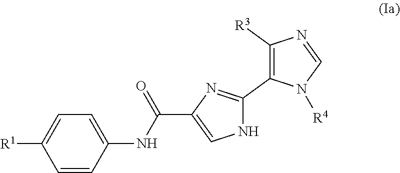

wherein:
R¹ is piperazine, wherein the piperazine is optionally substituted with one or more substituents independently selected at each occurrence from oxo, —S(O₂)NH₂, and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is unsubstituted C₁-C₆ alkyl;
wherein the method comprises reacting a compound represented by Formula (E-1), or a pharmaceutically acceptable salt thereof,

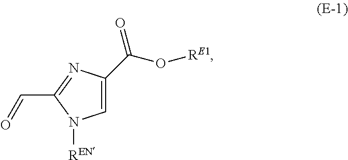

wherein,
   R^{EN′} is hydrogen or R^{EN}, and wherein R^{EN} is a protecting group;
   R^{E1} is C₁-C₆ alkyl (e.g., ethyl), C₁-C₆haloalkyl, C₁-C₆hydroxyalkyl, C₁-C₆aminoalkyl, C₁-C₆heteroalkyl, substituted or unsubstituted aryl, or substituted or unsubstituted heteroaryl;
with a compound represented by Formula (F), or a pharmaceutically acceptable salt thereof,

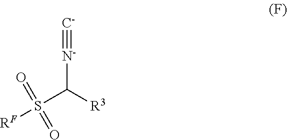

wherein R³ is defined above, and
R^{F} is a substituted or unsubstituted phenyl,
thereby producing a compound of Formula (G-1a), or a pharmaceutically acceptable salt thereof,

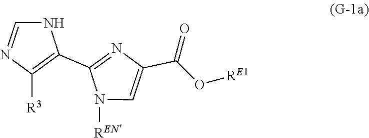

wherein R³, R^{EN} and R^{E1} are defined above,
   and wherein the method further comprises reacting the compound of Formula (G-1a), or a pharmaceutically acceptable salt thereof, with a compound of Formula (J), or a pharmaceutically acceptable salt thereof,

R⁴—R^{J} (J)

wherein R¹ is a leaving group,
thereby producing a compound represented by Formula (G-1), or a pharmaceutically acceptable salt thereof,

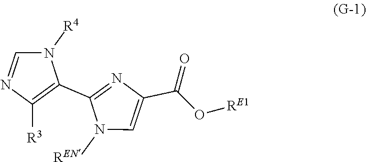

wherein R³, R⁴, R^{EN′} and R^{E1} are defined above.

In one aspect, provided herein is a compound represented by Formula (A), or a pharmaceutically acceptable salt thereof,

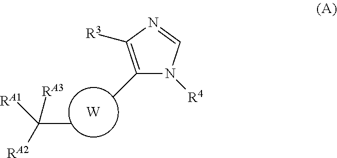

wherein
R³ is selected from optionally substituted C₁-C₆ alkyl, optionally substituted 3- to 10-membered heterocycle and optionally substituted C₃₋₁₀ carbocycle, wherein each of the alkyl, heterocycle and carbocycle is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R⁴ is selected from:
   hydrogen;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
   optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
Ring W is an optionally substituted 5 or 6-membered heteroaryl, wherein the 5 or 6-membered heteroaryl is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
each of R^{A1}, R^{A2} and R^{A3} is independently selected from a halogen and —O—C₁₋₆alkyl.

In certain embodiments, the disclosure provides a method of synthesizing a compound represented by Formula (I):

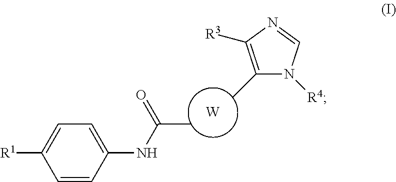

or a pharmaceutically acceptable salt thereof, wherein:
R¹ is selected from:
   —N(R⁵)₂, wherein R⁵ is selected from hydrogen, and optionally substituted C₁-C₆ alkyl, wherein the optional substituents on C₁-C₆ alkyl are independently selected at each occurrence from one or more halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
   substituted C₁-C₆ alkyl, wherein the substituents on C₁-C₆ alkyl are independently selected at each occurrence from one or more halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, 3- to 12-membered heterocycle;
   optionally substituted 3 to 8-membered heterocycle; wherein the optional substituents on the 3 to 8-membered heterocycle are independently selected at each occurrence from one or more halogen, —OH, —CN, —NO₂, —NH₂, oxo, ═S, —S(O₂)NH₂, —C₁₋₁₀ haloalkyl, —O—C₁₋₁₀ alkyl, C₂₋₁₀alkenyl, C₂₋₁₀ alkynyl, C₃₋₁₂ carbocycle, 3- to 12-membered heterocycle, and optionally substituted C₁₋₁₀ alkyl, wherein the optional substituents on the C₁₋₁₀ alkyl are independently selected at each occurrence from one or more hydroxy, halogen, oxo, —C₁₋₄₀ haloalkyl, —NH₂, —CN, and —NO₂;
R³ is selected from optionally substituted C₁-C₆ alkyl, optionally substituted 3- to 10-membered heterocycle and optionally substituted C₃₋₁₀ carbocycle, wherein the optional substituents on each are independently selected at each occurrence from one or more halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
R⁴ is selected from:
   hydrogen;
   optionally substituted C₁-C₆ alkyl, wherein the optional substituents on C₁-C₆ alkyl are independently selected at each occurrence from one or more halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
   optionally substituted C₃₋₁₀ carbocycle, wherein the optional substituents on C₃₋₁₀ carbocycle are independently selected at each occurrence from one or more halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle; and
W is selected from optionally substituted 5- to 6-membered heteroaryl, wherein the substituents on the optionally substituted 5- to 8-membered heteroaryl are independently selected at each occurrence from one or more halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle.

In some aspects, the disclosure provides a method of treating or preventing disease comprising administering a compound synthesized by a method described herein (e.g., a compound of Formula (Ia) or Formula (I) or a salt thereof) and a pharmaceutically acceptable excipient to a subject in need thereof. In some aspects, the disease is a cancer. In some cases, the cancer is selected from colorectal cancer, gastric cancer, breast cancer, lung cancer, pancreatic cancer, prostate cancer, multiple myeloma, chronic myelogenous leukemia, cancer metastasis, fibrosis and psychiatric disorders. In some cases, the pharmaceutical composition can be used as an inhibitor of tumor immunosuppression in combination with chemotherapy or immune checkpoint inhibitor therapy for cancer. In some cases, the pharmaceutical composition can be used to treat a fibrotic disease or condition including but not limited to chronic kidney fibrosis (“CKD”), liver cirrhosis, pulmonary fibrosis, renal interstitial fibrosis, myocardial infarction, skin fibrosis, systemic sclerosis (“SSc”), and graft-versus-host disease (“GVHD”). In some cases, the pharmaceutical composition can be used to treat kidney fibrosis. In some cases, the pharmaceutical composition can be used to treat skin fibrosis. In some cases, the pharmaceutical composition can be used to treat idiopathic pulmonary fibrosis (IPF). In some cases, the pharmaceutical composition can be used to treat a disease is associated with TNIK kinase.

In some aspects, the disclosure provides a method of inhibiting TNIK kinase comprising administering a compound synthesized by a method described herein (e.g., a compound of Formula (Ia) or Formula (I) or a salt thereof) and a pharmaceutically acceptable excipient to a subject in need thereof.

In some aspects, the disclosure provides a method of inhibiting MAP4K4 kinase comprising administering a compound synthesized by a method described herein (e.g., a compound of Formula (Ia) or Formula (I) or a salt thereof) and a pharmaceutically acceptable excipient to a subject in need thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

### A. DEFINITIONS

Unless defined otherwise, all technical and scientific terms used herein have the same meaning as is commonly understood by one of skill in the art to which this disclosure belongs. All patents and publications referred to herein are incorporated by reference.

“Alkyl” refers to a straight or branched hydrocarbon chain radical consisting solely of carbon and hydrogen atoms, containing no unsaturation, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, -COOMe, —OH, -OMe, —NH₂, or —NO₂. In some embodiments, the alkyl is optionally substituted with halogen, —CN, —OH, or -OMe. In some embodiments, the alkyl is optionally substituted with halogen.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to an alkyl group that may consist of 1 carbon atom, 2 carbon atoms, 3 carbon atoms, 4 carbon atoms, 5 carbon atoms or 6 carbon atoms, including straight-chain alkyl and branched-chain alkyl groups.

“Alkoxy” refers to a radical bonded through an oxygen atom of the formula —O-alkyl, where alkyl is an alkyl chain as defined above. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₅ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively. The term —C_{x-y}alkenylene- refers to a substituted or unsubstituted alkenylene chain with from x to y carbons in the alkenylene chain. For example, —C₂-(alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkenylene chain. For example, —C₂₋₆alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” or “alkylene chain” refers to a straight or branched divalent hydrocarbon chain linking the rest of the molecule to a radical group, consisting solely of carbon and hydrogen, containing no unsaturation, and preferably having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group may be through any two carbons within the chain. In certain embodiments, an alkylene comprises one to ten carbon atoms (i.e., C₁-C₈ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). The term —C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

“Alkenylene” or “alkenylene chain” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group may be through any two carbons within the chain. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group may be through any two carbons within the chain. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

“Aryl” refers to a radical derived from an aromatic monocyclic or aromatic multicyclic hydrocarbon ring system by removing a hydrogen atom from a ring carbon atom, wherein the ring system contains at least one aromatic ring. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hükel theory. The ring system from which aryl groups are derived include, but are not limited to, groups such as benzene, fluorene, indane, indene, tetralin and naphthalene. The aryl radical may be a monocyclic, bicyclic, tricyclic or tetracyclic ring system, which may include fused or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Aralkyl” refers to a radical of the formula —R^{c}-aryl where R^{c} is an alkylene chain as defined above, for example, methylene, ethylene, and the like.

“Aralkenyl” refers to a radical of the formula —R^{d}-aryl where R^{d} is an alkenylene chain as defined above. “Aralkynyl” refers to a radical of the formula —R^{e}-aryl, where R^{e} is an alkynylene chain as defined above.

“Carbocycle” refers to a saturated, unsaturated or aromatic ring system in which each ring atom of the ring system is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. An aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. In some embodiments, the carbocycle is an aryl. In some embodiments, the carbocycle is a cycloalkyl. In some embodiments, the carbocycle is a cycloalkenyl. In some embodiments, the carbocycle contains a triple bond. Unless stated otherwise specifically in the specification, a carbocycle can be optionally substituted.

“Cycloalkyl” refers to a fully saturated monocyclic or polycyclic hydrocarbon radical consisting solely of carbon and hydrogen atoms, which includes fused or bridged ring systems, and preferably having from three to twelve carbon atoms. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Heterocycloalkyl” refers to a cycloalkyl group, as defined above, wherein one or more ring carbons are replaced with one or more heteroatoms, such as N, O, P, and S. A heterocycloalkyl may be optionally substituted.

“Cycloalkenyl” refers to an unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond. In certain embodiments, a cycloalkenyl comprises one double bond. In certain embodiments, a cycloalkenyl comprises more than one double bond. In certain embodiments, a cycloalkenyl comprises three to ten carbon atoms. In other embodiments, a cycloalkenyl comprises five to seven carbon atoms. The cycloalkenyl may be attached to the rest of the molecule by a single bond. Examples of monocyclic cycloalkenyls includes, e.g., cyclopentenyl, cyclohexenyl, cycloheptenyl, and cyclooctenyl.

“Heterocycloalkenyl” refers to a cycloalkenyl group, as defined above, wherein one or more ring carbons are replaced with one or more heteroatoms, such as N, O, P, and S. A heterocycloalkenyl may be optionally substituted.

“Cycloalkylalkyl” refers to a radical of the formula —R^{c}-cycloalkyl where R^{c} is an alkylene chain as described above.

“Cycloalkylalkoxy” refers to a radical bonded through an oxygen atom of the formula —O—R^{c}-cycloalkyl where R^{c} is an alkylene chain as described above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc.). When an alkyl group is substituted with more than one halogen radicals, each halogen may be independently selected e.g., 1-chloro,2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Heterocycle” refers to a saturated, unsaturated or aromatic ring comprising one or more heteroatoms. Exemplary heteroatoms include N, O, Si, P, B, and S atoms. Heterocycles include e.g., 3- to 10-membered monocyclic rings, 6- to 12-membered bicyclic rings, and 6- to 12-membered bridged rings. Each ring of a bicyclic heterocycle may be selected from saturated, unsaturated, and aromatic rings. “Heterocyclene” refers to a divalent heterocycle linking the rest of the molecule to a radical group. Unless stated otherwise specifically in the specification, a heterocycle is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycle is optionally substituted with halogen, methyl, ethyl, —CN, —CF₃, —OH, or —OMe. In some embodiments, the heterocycloalkyl is optionally substituted with halogen. In some embodiments, a heterocycle is a heteroaryl. In some embodiments, a heterocycle is a heterocycloalkyl. In some embodiments, a heterocycle is a heterocycloalkenyl. In some embodiments, a heterocycle contains one or more triple bonds.

In some embodiments, the heterocycle comprises one to three heteroatoms selected from the group consisting of nitrogen, oxygen, and sulfur. In some embodiments, the heterocycle comprises one to three heteroatoms selected from the group consisting of nitrogen and oxygen. In some embodiments, the heterocycle comprises one to three nitrogens. In some embodiments, the heterocycle comprises one or two nitrogens. In some embodiments, the heterocycle comprises one nitrogen. In some embodiments, the heterocycle comprises one nitrogen and one oxygen. Unless stated otherwise specifically in the specification, the heterocycle radical may be a monocyclic, bicyclic, tricyclic or tetracyclic ring system, which may include fused, spiro, or bridged ring systems; and the nitrogen, carbon, or sulfur atoms in the heterocycle radical may be optionally oxidized; the nitrogen atom may be optionally quaternized. Representative heterocycles include, the heteroaryl groups described below. Representative heterocycle also include, but are not limited to, heterocycles having from two to fifteen carbon atoms (C₂-C₁₅ heterocycloalkyl or C₂-C₁₅ heterocycloalkenyl), from two to ten carbon atoms (C₂-C₁₀ heterocycloalkyl or C₂-C₁₀ heterocycloalkenyl), from two to eight carbon atoms (C₂-C₈ heterocycloalkyl or C₂-C₈ heterocycloalkenyl), from two to seven carbon atoms (C₂-C₇ heterocycloalkyl or C₂-C₇ heterocycloalkenyl), from two to six carbon atoms (C₂-C₆ heterocycloalkyl or C₂-C₇ heterocycloalkenyl), from two to five carbon atoms (C₂-C₅ heterocycloalkyl or C₂-C₅ heterocycloalkenyl), or two to four carbon atoms (C₂-C₄ heterocycloalkyl or C₂-C₄ heterocycloalkenyl). Examples of such heterocycle radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofumyl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycle also includes all ring forms of the carbohydrates, including but not limited to the monosaccharides, the disaccharides and the oligosaccharides. In some embodiments, heterocycles have from 2 to 10 carbons in the ring. It is understood that when referring to the number of carbon atoms in a heterocycle, the number of carbon atoms in the heterocycle is not the same as the total number of atoms (including the heteroatoms) that make up the heterocycle (i.e. skeletal atoms of the heterocycle ring). In some embodiments, the heterocycle is a 3- to 8-membered. In some embodiments, the heterocycle is a 3- to 7-membered. In some embodiments, the heterocycle is a 3- to 6-membered. In some embodiments, the heterocycle is a 4- to 6-membered. In some embodiments, the heterocycle is a 5- to 6-membered.

“Heteroaryl” or “aromatic heterocycle” refers to a radical derived from a heteroaromatic ring radical that comprises one to thirteen carbon atoms, at least one heteroatom wherein each heteroatom may be selected from N, O, and S, and at least one aromatic ring. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the HG-kel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, pyridine, pyrimidine, oxazole, furan, thiophene, benzthiazole, and imdazopyridine. An “X-membered heteroaryl” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a},
—R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N (R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, and heterocycle, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a},
—R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N (R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, and heterocycle, wherein each R, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R—N(R^{a})₂, —R^{b}—C(O)R^{a}, —R^{b}—C(O)O R^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

As used in the specification and claims, the singular form “a”, “an” and “the” includes plural references unless the context clearly dictates otherwise.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

The terms “treat,” “treating” or “treatment,” as used herein, may include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### B. METHODS OF SYNTHESIS AND COMPOUNDS

In one aspect, provided herein is a method of producing a compound represented by Formula (I), or a pharmaceutically acceptable salt thereof,

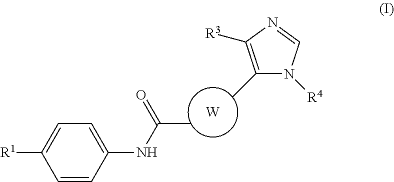

wherein:
R¹ is selected from:
N(R⁵)₂, wherein each R⁵ is independently selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle; and
optionally substituted 3 to 8-membered heterocycle; wherein the 3 to 8-membered heterocycle is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, and —NO₂;
R³ is selected from optionally substituted C₁-C₆ alkyl, optionally substituted 3- to 10-membered heterocycle and optionally substituted C₃₋₁₀ carbocycle, wherein each of the alkyl, heterocycle and carbocycle is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R⁴ is selected from:
hydrogen;
optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
Ring W is an optionally substituted 5 or 6-membered heteroaryl, wherein the 5 or 6-membered heteroaryl is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle,
wherein the method comprises subjecting a compound represented by Formula (A), or a pharmaceutically acceptable salt thereof,

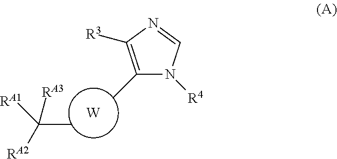

wherein Ring W, R³ and R⁴ are defined above, and
each of R^{A}, R^{A2} and R^{A3} is independently selected from a halogen and —O—C₁₋₆alkyl;
through a hydrolysis step to produce a compound represented by Formula (B), or a pharmaceutically acceptable salt thereof,

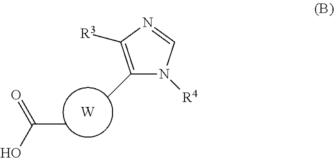

wherein Ring W, R³ and R⁴ are defined above.

In one aspect, provided herein is a method of producing a compound represented by Formula (B), or a pharmaceutically acceptable salt thereof,

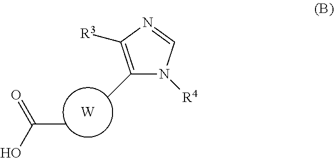

wherein:
R³ is selected from optionally substituted C₁-C₆ alkyl, optionally substituted 3- to 10-membered heterocycle and optionally substituted C₃₋₁₀ carbocycle, wherein each of the alkyl, heterocycle and carbocycle is optionally substituted with one or more substituents independently selected at each occurrence from halogen, —OH, —CN, —NO₂, —NH₂, oxo, ═S, C₁₋₆ alkyl, -Coo haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R⁴ is selected from:
   hydrogen;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
   optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
Ring W is an optionally substituted 5 or 6-membered heteroaryl, wherein the 5 or 6-membered heteroaryl is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle,
wherein the method comprises subjecting a compound represented by Formula (A), or a pharmaceutically acceptable salt thereof,

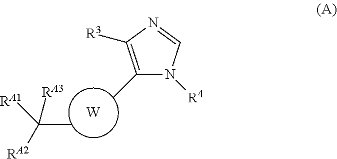

wherein Ring W, R³ and R⁴ are defined above, and
each of R^{A1}, R^{A2} and R^{A3} is independently selected from a halogen and —O—C₁₋₆alkyl;
through a hydrolysis step, thereby producing the compound represented by Formula (B), or a pharmaceutically acceptable salt thereof.

In some embodiments of Formula (A), each of R^{A1}, R^{A2} and R^{A3} is independently selected from halogen and —O—C₁₋₃alkyl. In some embodiments, each of R^{A1}, R^{A2} and R^{A3} is independently selected from a halogen. In some embodiments, each of R^{A1}, R^{A2} and R^{A3} is independently selected from a —O—C₁₋₆alkyl. In some embodiments, each of R^{A1}, R^{A2} and R^{A3} is independently selected from F, Cl, Br, OMe, and OEt. In some embodiments,

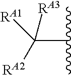

is CBr₃, CCl₃, CF₃, CF₂Cl, CFCl₂, CF₂Br, CFBr₂, C(OMe)₃, or C(OEt)₃. In some embodiments,

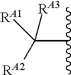

is CF₃. In some embodiments, the hydrolysis step is performed in a solvent selected from ether, alcohol, water, and a mixture thereof. (e.g., MeOH/Water). In some embodiments, the hydrolysis step is performed in a solvent selected from 1,4-dioxane, MeOH, EtOH, i-PrOH, n-PrOH, THF, water, or a mixture thereof. In some embodiments, the hydrolysis step is performed in a mixture of methanol and water. In some embodiments, the hydrolysis step is performed in an alkaline condition. In some embodiments, the hydrolysis step is performed in the presence of a base, wherein the base is an alkali, an alkaline salt, or a mixture thereof. In some embodiments, the alkaline salt is a carbonate salt, bicarbonate salt, or phosphate salt (e.g., phosphate, hydrogenphosphate and dihydrogenphosphate). In some embodiments, the base is selected from NaOH, KOH, LiOH, Ca(OH)₂, K₂CO₃, KHCO₃, Na₂CO₃, NaHCO₃, K₃PO₄, K₂HPO₄, KH₂PO₄, Na₃PO₄, Na₂HPO₄, NaH₂PO₄, or a combination thereof. In some embodiments, the base is NaOH.

In some embodiments, the method further comprises subjecting the compound represented by Formula (B), or a pharmaceutically acceptable salt thereof, to a condensation reaction with a compound of Formula (C), or a pharmaceutically acceptable salt thereof,

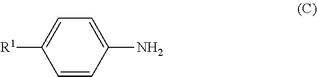

wherein R¹ is in Formula (I). In some embodiments, the condensation step is performed in the presence of a base (e.g., DIEA), a coupling agent (HATU), or both. In some embodiments, the condensation reaction is conducted in a polar solvent. In some embodiments, the condensation reaction is conducted in either. In some embodiments, the condensation reaction is conducted in THF. In some embodiments, the condensation reaction is conducted at a temperature at 15-25 °C. In some embodiments, the condensation reaction is conducted at a temperature at 0-40 °C.

In some embodiments, the compound represented by Formula (I) has a structure of Formula (Ia),

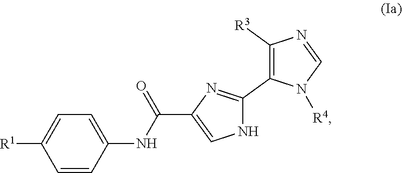

wherein:
R¹ is piperazine, wherein the piperazine is optionally substituted with one or more substituents independently selected at each occurrence from oxo and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is substituted C₁-C₆ alkyl, wherein the C₁-C₆ alkyl is substituted with one or more halogen.

In some embodiments, the compound represented by Formula (I) has a structure of Formula (Ia),

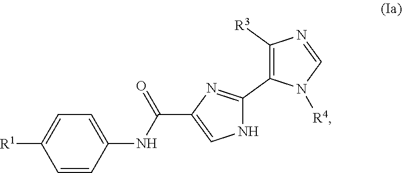

wherein,
R¹ is piperazine, wherein the piperazine is optionally substituted with one or more substituents independently selected at each occurrence from oxo, —S(O₂)NH₂, and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is unsubstituted C₁-C₆ alkyl.

In one aspect, provided herein is a method of producing a compound represented by Formula (I), or a pharmaceutically acceptable salt thereof,

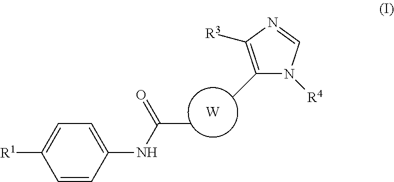

wherein:
R¹ is selected from:
   N(R⁵)₂, wherein each R⁵ is independently selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle; and
   optionally substituted 3 to 8-membered heterocycle; wherein the 3 to 8-membered heterocycle is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, and —NO₂;
R³ is selected from optionally substituted C₁-C₆ alkyl, optionally substituted 3- to 10-membered heterocycle and optionally substituted C₃₋₁₀ carbocycle, wherein each of the alkyl, heterocycle and carbocycle is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R⁴ is selected from:
   hydrogen;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
   optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
Ring W is an optionally substituted 5 or 6-membered heteroaryl, wherein the 5 or 6-membered heteroaryl is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle,
wherein the method comprises reacting a compound represented by Formula (E), or a pharmaceutically acceptable salt thereof,

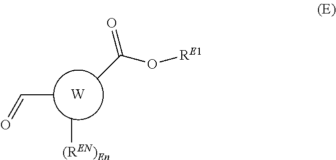

wherein,
   Ring W is defined above,
   En is 0 or 1,
   R^{EN} is a protecting group, and
   R^{E1} is C₁-C₆ alkyl (e.g., ethyl), C₁-C₆haloalkyl, C₁-C₆hydroxyalkyl, C₁-C₆aminoalkyl, C₁-C₆heteroalkyl, substituted or unsubstituted aryl, or substituted or unsubstituted heteroaryl;
with a compound represented by Formula (F), or a pharmaceutically acceptable salt thereof,

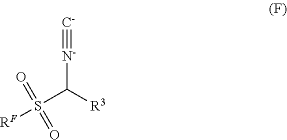

wherein R³ is defined above, and
R^{F} is a substituted or unsubstituted phenyl,
   in the presence of a compound of Formula (K), or a pharmaceutically acceptable salt thereof,

R⁴—NH₂ (K),

thereby producing a compound represented by Formula (G), or a pharmaceutically acceptable salt thereof,

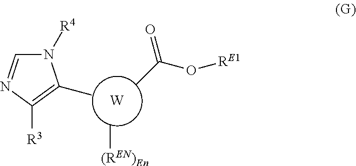

wherein R³, R⁴, Ring W, En, R^{EN} and R^{E1} are defined above.

In one aspect, provided herein is a method of producing a compound represented by Formula (G), or a pharmaceutically acceptable salt thereof,

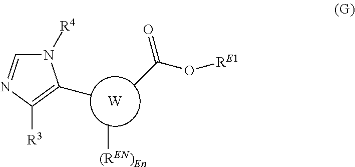

wherein:
R³ is selected from optionally substituted C₁-C₆ alkyl, optionally substituted 3- to 10-membered heterocycle and optionally substituted C₃₋₁₀ carbocycle, wherein each of the alkyl, heterocycle and carbocycle is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R⁴ is selected from:
   hydrogen;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
   optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and Ring W is an optionally substituted 5 or 6-membered heteroaryl, wherein the 5 or 6-membered heteroaryl is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
En is 0 or 1;
R^{EN} is a protecting group; and
R^{E1} is C₁-C₆ alkyl (e.g., ethyl), C₁-C₆haloalkyl, C₁-C₆hydroxyalkyl, C₁-C₆aminoalkyl, C₁-C₆heteroalkyl, substituted or unsubstituted aryl, or substituted or unsubstituted heteroaryl;
wherein the method comprises reacting a compound represented by Formula (E), or a pharmaceutically acceptable salt thereof,

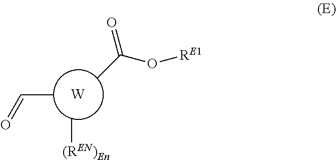

wherein,
   Ring W, En, R^{EN}, and R^{E1} are defined above,
with a compound represented by Formula (F), or a pharmaceutically acceptable salt thereof,

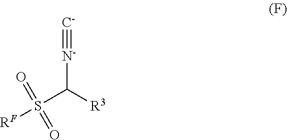

wherein R³ is defined above, and
R^{F} is a substituted or unsubstituted phenyl,
   in the presence of a compound of Formula (K), or a pharmaceutically acceptable salt thereof,

R⁴—NH₂ (K),
thereby producing a compound represented by Formula (G), or a pharmaceutically acceptable salt thereof.

In some embodiments of Formulas (E), (E-1), (G), (G-a), (G-1), and (G-1a), R^{E1} is methyl, ethyl, n-propyl, i-propyl, n-butyl, sec-butyl, i-butyl, or t-butyl. In some embodiments, R^{E1} is ethyl. In some embodiments, R^{E1} is phenyl, benzyl, or p-methoxybenzyl. In some embodiments, R^{E1} is substituted or unsubstituted phenyl. In some embodiments, R^{E1} is substituted or unsubstituted heteroaryl. In some embodiments, R^{E1} is optionally substituted with one or more substituents selected from halogen, —NH₂, CN, NO₂, —OH, —SH, SF₅, C₁-C₆alkyl, —OC₁-C₆alkyl, C₁-C₆haloalkyl, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆hydroxyalkyl, C₁-C₆aminoalkyl, and C₁-C₆heteroalkyl. In some embodiments, R^{E1} is optionally substituted with one or more substituents selected from halogen, —NH₂, CN, NO₂, —OH, —C(═O)OH, —SH, SF₅, C₁-C₅alkyl, —OC₁-C₆alkyl, C₁-C₆haloalkyl, and —OC₁-C₆alkyl.

In some embodiments, En is 0. In some embodiments, En is 1.

In some embodiments, the compound of Formula (E) has a structure of Formula (E-1),

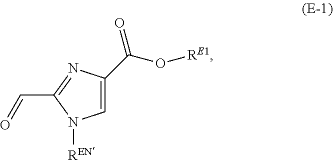

wherein R^{EN′} is hydrogen or R^{EN}. In some embodiments, the compound of Formula (E-1) has a structure of

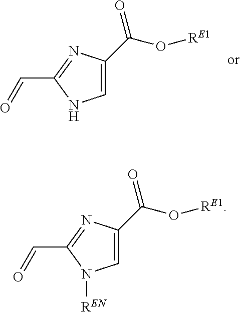

In some embodiments, the compound of Formula (E-1) has a structure of

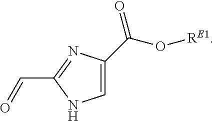

In some embodiments, the compound of Formula (E-1) has a structure of

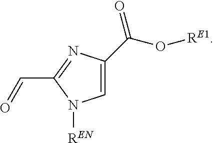

In some embodiments of Formulas (E), (G), (G-a), and (H), R^{EN} is selected from (trimethyl silicon)ethoxymethyl (SEM), methyloxycarbonyl, ethyloxycarbonyl, benzyloxycarbonyl (CBz), tert-butoxycarbonyl (Boc), 9-fluorenylmethyloxycarbonyl (Fmoc), allyloxycarbonyl (Alloc), 2-(trimethylsilyl)ethyloxycarbonyl (Teoc), 2,2,2-trichloroethoxycarbonyl (Troc), para-toluenesulfonyl (Tos), 2,2,2-trifluoroacetyl (Tfa), trityl (Trt), 2,4-dimethocybenzyl (Dmb), p-Methoxybenzyl (Pmb), and benzyl (Bn). In some embodiments, R^{EN} is SEM.

In some embodiments of Formula (F), R^{F} is optionally substituted with one or more R^{F1}, and each R^{F1} is independent selected from halogen, —NH₂, CN, NO₂, —OH, —SH, SF₅, C₁-C₆alkyl, —OC₁-C₆alkyl, C₁-C₆haloalkyl, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₅alkyl, C₁-C₆hydroxyalkyl, C₁-C₆aminoalkyl, and C₁-C₆heteroalkyl.

In some embodiments of Formula (F), R^{F} is optionally substituted with one or more R^{F1}, and each R^{F1} is independent selected from F, Cl, Br, methyl, and methoxy.

In some embodiments, the compound of Formula (F) has a structure of Formula (F-1)

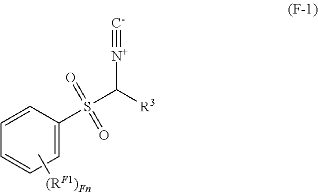

wherein Fn is 0, 1, 2, 3, 4, or 5, and each R^{F1} is independent selected from halogen, —NH₂, CN, NO₂, —OH, —SH, SF₅, C₁-C₆alkyl, —OC₁-C₆alkyl, C₁-C₆haloalkyl, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆hydroxyalkyl, C₁-C₆aminoalkyl, and C₁-C₆heteroalkyl. In some embodiments, the compound of Formula (F) is

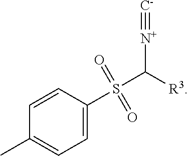

In some embodiments, the compound represented by Formula (E), Formula (E-1) or a salt thereof, and the compound represented by Formula (F) or a salt thereof is reacted in a commercially available solvent. In some embodiments, the commercially available solvent is THF, 2-MeTHF, toluene, 1,4-dioxane, 1,2-DCE, DCM, DMF, DMAc, NMP, DMSO, acetone, acetonitrile, EtOH, MeOH, i-PrOH, t-BuOH, etc or their combination.

In some embodiments, the compound represented by Formula (G) has a structure of Formula (G-1)

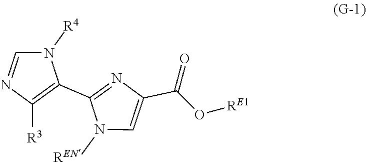

wherein R^{EN′} is hydrogen or R^{EN}.

In some embodiments, the compound represented by Formula (G-1) has a structure of

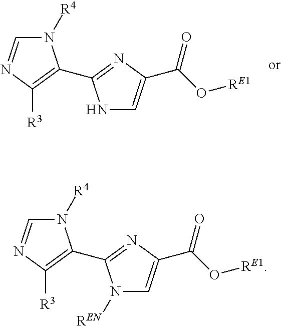

In some embodiments, the compound represented by Formula (G-1) has a structure of

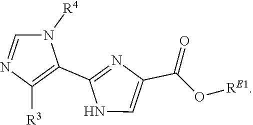

In some embodiments, the compound represented by Formula (G-1) has a structure of

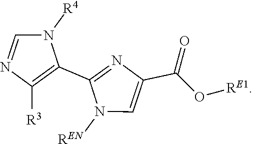

In some embodiments, the method further comprises subjecting the compound represented by Formula (G) or a pharmaceutically acceptable salt thereof, to a hydrolysis reaction, thereby producing a compound of Formula (H), or a pharmaceutically acceptable salt thereof,

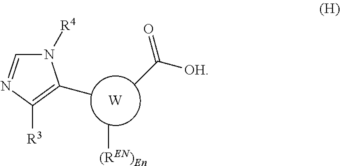

In some embodiments, the compound represented by Formula (H) has a structure of Formula (H-1),

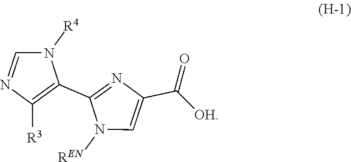

In some embodiments, the compound represented by Formula (H-1) has a structure of

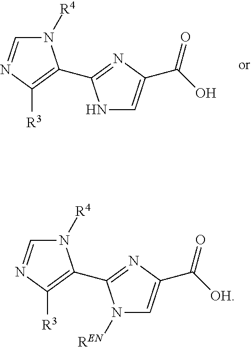

In some embodiments, the method further comprises subjecting the compound represented by Formula (H) or a pharmaceutically acceptable salt thereof, to a condensation reaction with a compound of Formula (C), or a pharmaceutically acceptable salt thereof,

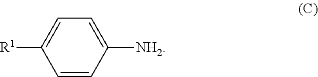

In some embodiments, further comprising a deprotecting reaction. In some embodiments, the deprotecting reaction comprises replacing R^{EN} with a hydrogen. In some embodiments, the deprotecting reaction occurs after the condensation reaction. In some embodiments, the deprotecting reaction occurs before the condensation reaction

In some embodiments, the compound represented by Formula (I) has a structure of Formula (Ta),

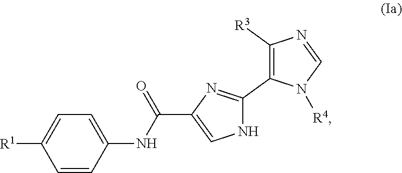

wherein:
R¹ is piperazine, wherein the piperazine is optionally substituted with one or more substituents independently selected at each occurrence from oxo and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is substituted C₁-C₆ alkyl, wherein the C₁-C₆ alkyl is substituted with one or more halogen.

In some embodiments, the compound represented by Formula (I) has a structure of Formula (Ta),

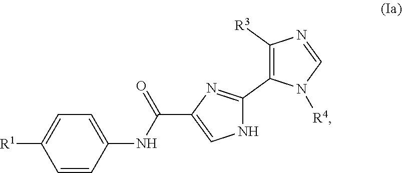

wherein,
R¹ is piperazine, wherein the piperazine is optionally substituted with one or more substituents independently selected at each occurrence from oxo, —S(O₂)NH₂, and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is unsubstituted C₁-C₆ alkyl.

In one aspect, provided herein is a method of producing a compound represented by Formula (I), or a pharmaceutically acceptable salt thereof,

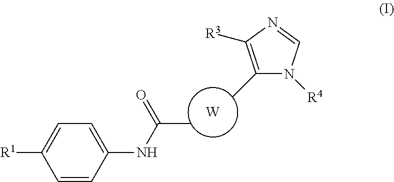

wherein:
R¹ is selected from:
   N(R⁵)₂, wherein each R⁵ is independently selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle; and
   optionally substituted 3 to 8-membered heterocycle; wherein the 3 to 8-membered heterocycle is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀haloalkyl, —NH₂, —CN, and —NO₂;
R³ is selected from optionally substituted C₁-C₆ alkyl, optionally substituted 3- to 10-membered heterocycle and optionally substituted C₃₋₁₀ carbocycle, wherein each of the alkyl, heterocycle and carbocycle is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
R⁴ is selected from:
   hydrogen;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
   optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀alkenyl, C₂₋₁₀ alkynyl, C₃₋₁₂ carbocycle, and 3- to 12-membered heterocycle;
Ring W is an optionally substituted 5 or 6-membered heteroaryl, wherein the 5 or 6-membered heteroaryl is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle,
wherein the method comprises reacting a compound represented by Formula (E), or a pharmaceutically acceptable salt thereof,

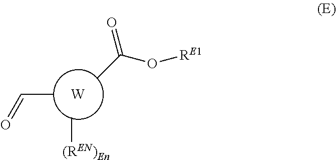

wherein,
   Ring W is defined above,
   En is 0,
   R^{EN} is a protecting group, and
   R^{E1} is C₁-C₆ alkyl (e.g., ethyl), C₁-C₆haloalkyl, C₁-C₆hydroxyalkyl, C₁-C₆aminoalkyl, C₁-C₆heteroalkyl, substituted or unsubstituted aryl, or substituted or unsubstituted heteroaryl;
with a compound represented by Formula (F), or a pharmaceutically acceptable salt thereof,

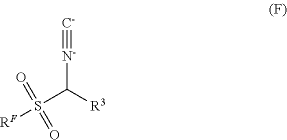

wherein R³ is defined above, and
R^{F} is a substituted or unsubstituted phenyl,
thereby producing a compound of Formula (G-a), or a pharmaceutically acceptable salt thereof,

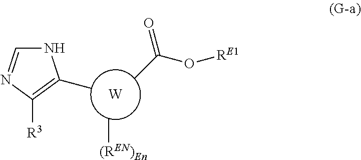

wherein R³, Ring W, En, R^{EN} and R^{E1} are defined above,
and wherein the method further comprises reacting the compound of Formula (G-a), or a pharmaceutically acceptable salt thereof, with a compound of Formula (J), or a pharmaceutically acceptable salt thereof,

R⁴—R^{J} (J)
wherein R^{J} is a leaving group and R⁴ is defined above,
thereby producing a compound represented by Formula (G), or a pharmaceutically acceptable salt thereof,

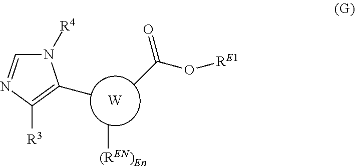

wherein R³, R⁴, R^{EN}, En, and R^{E1} are defined above.

In some embodiments, the compound represented by Formula (E), or a pharmaceutically acceptable salt thereof and the compound represented by Formula (F), or a pharmaceutically acceptable salt thereof are reacted in the presence of NH₃ gas or NH₃ in solvent, e.g., NH₃·H₂O, NH₃ in MeOH, NH₃ in EtOH and other NH₃ solution to produce the compound of Formula (G-a), or a pharmaceutically acceptable salt thereof. In some embodiments, the NH₃ is present in a solvent such as alcohol and water.

In some embodiments, the compound of Formula (E) has a structure of

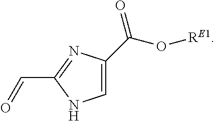

In some embodiments, the compound of Formula (G-a) has a structure of

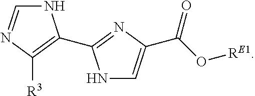

In some embodiments, the compound of Formula (G) has a structure of

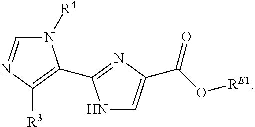

In some embodiments of Formula (J), R^{J} is a halogen (e.g., Br, Cl, I). In some embodiments, R^{J} is alkylsulfonate (e.g., trifluoromethanesulfonate (triflate or TfO⁻)) or arylsulfonate (e.g., 4-methylbenzenesulfonate). In some embodiments, R^{J} is Br. In some embodiments, R^{J} is Cl. In some embodiments, R^{J} is I. In some embodiments, R^{J} is triflate. In some embodiments, R^{J} is 4-methylbenzenesulfonate.

In some embodiments, R⁴—R^{J} is 2-bromopropane. In some embodiments, R⁴—R^{J} is 2-chloropropane. In some embodiments, R⁴—R^{J} is 2-iodopropane. In some embodiments, R⁴—R^{J} is isopropyl trifluoromethanesulfonate. In some embodiments, R⁴—R^{J} is isopropyl 4-methylbenzenesulfonate.

In some embodiments, R⁴—R^{J} is 2-chlorine-1,1,1-trifluoroethane. In some embodiments, R⁴—R^{J} is 2-Bromo-1,1,1-trifluoroethane. In some embodiments, R⁴—R^{J} is 2-iodo-1,1,1-trifluoroethane. In some embodiments, R⁴—R^{J} is 2,2,2-Trifluoroethyl p-toluenesulfonate.

In some embodiments, the method further comprises a deprotecting reaction. In some embodiments, the deprotecting reaction occurs before the reacting of the compound represented by Formula (E) with the compound represented by Formula (F). In some embodiments, the compound represented by Formula (E) is deprotected.

In some embodiments, the method further comprises subjecting the compound represented by Formula (G) or a pharmaceutically acceptable salt thereof, to a hydrolysis reaction, thereby producing a compound of Formula (H), or a pharmaceutically acceptable salt thereof,

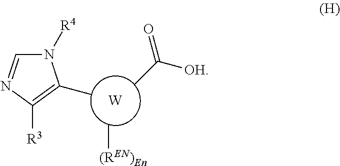

In some embodiments, the compound of Formula (H) has a structure of

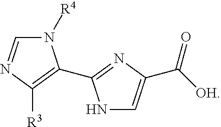

In some embodiments, the method further comprises subjecting the compound represented by Formula (H) or a pharmaceutically acceptable salt thereof, to a condensation reaction with a compound of Formula (C), or a pharmaceutically acceptable salt thereof,

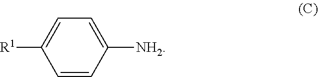

In some embodiments, the compound represented by Formula (I) has a structure of Formula (Ia),

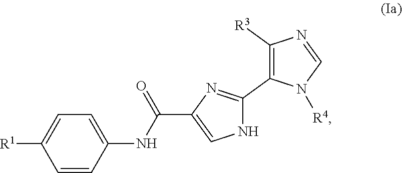

wherein:
R¹ is piperazine, wherein the piperazine is optionally substituted with one or more substituents independently selected at each occurrence from oxo and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is substituted C₁-C₆ alkyl, wherein the C₁-C₆ alkyl is substituted with one or more halogen.

In some embodiments, the compound represented by Formula (I) has a structure of Formula (Ia),

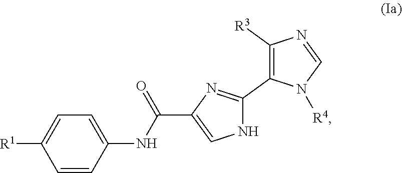

wherein,
R¹ is piperazine, wherein the piperazine is optionally substituted with one or more substituents independently selected at each occurrence from oxo, —S(O₂)NH₂, and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is unsubstituted C₁-C₆ alkyl.

In one aspect, provided herein is a method of producing a compound represented by Formula (Ta), or a pharmaceutically acceptable salt thereof,

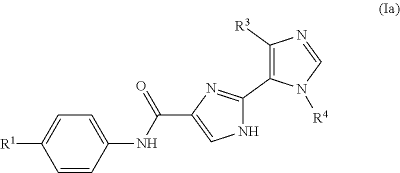

wherein:
R¹ is piperazine, wherein the piperazine is optionally substituted with one or more substituents independently selected at each occurrence from oxo, —S(O₂)NH₂, and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is unsubstituted C₁-C₆ alkyl;
wherein the method comprises reacting a compound represented by Formula (E-1), or a pharmaceutically acceptable salt thereof,

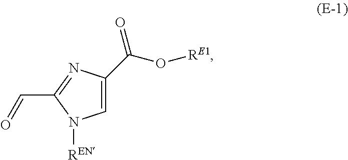

wherein,
   R^{EN′} is hydrogen or R^{EN}, and wherein R^{EN} is a protecting group;
R^{E1} is C₁-C₆ alkyl (e.g., ethyl), C₁-C₆haloalkyl, C₁-C₆hydroxyalkyl, C₁-C₆aminoalkyl, C₁-C₆heteroalkyl, substituted or unsubstituted aryl, or substituted or unsubstituted heteroaryl;
with a compound represented by Formula (F), or a pharmaceutically acceptable salt thereof,

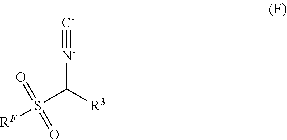

wherein R³ is defined above, and
R^{F} is a substituted or unsubstituted phenyl,
thereby producing a compound of Formula (G-1a), or a pharmaceutically acceptable salt thereof,

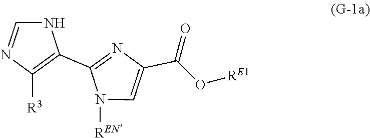

wherein R³, R^{EN′} and R^{E1} are defined above,
   and wherein the method further comprises reacting the compound of Formula (G-1a), or a pharmaceutically acceptable salt thereof, with a compound of Formula (J), or a pharmaceutically acceptable salt thereof,

R⁴—R^{J} (J)

wherein R is a leaving group,
thereby producing a compound represented by Formula (G-1), or a pharmaceutically acceptable salt thereof,

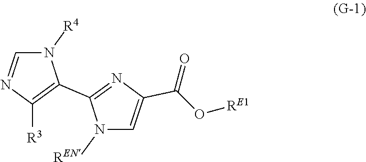

wherein R³, R⁴, R^{EN} and R^{E1} are defined above.

In some embodiments, the compound represented by Formula (E-1), or a pharmaceutically acceptable salt thereof and the compound represented by Formula (F), or a pharmaceutically acceptable salt thereof are reacted in the presence of NH₃ gas or NH₃ in solvent, e.g., NH₃·H₂O, NH₃ in MeOH, NH₃ in EtOH and other NH₃ solution to produce the compound of Formula (G-1a), or a pharmaceutically acceptable salt thereof. In some embodiments, the NH₃ is present in a solvent such as alcohol and water.

In some embodiments, the method further comprises subjecting the compound represented by Formula (G-1) or a pharmaceutically acceptable salt thereof, to a hydrolysis reaction, thereby producing a compound of Formula (H-1), or a pharmaceutically acceptable salt thereof,

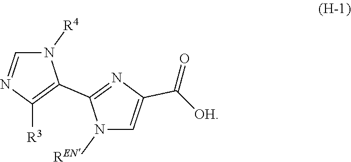

In some embodiments, the method further comprises subjecting the compound represented by Formula (H-1) or a pharmaceutically acceptable salt thereof, to a condensation reaction with a compound of Formula (C), or a pharmaceutically acceptable salt thereof,

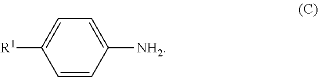

In some embodiments, R^{EN′} is hydrogen. In some embodiments, R^{EN′} is R^{EN}. In some embodiments, R^{EN′} is selected from (trimethyl silicon)ethoxymethyl (SEM), methyloxycarbonyl, ethyloxycarbonyl, benzyloxycarbonyl (CBz), tert-butoxycarbonyl (Boc), 9-fluorenylmethyloxycarbonyl (Fmoc), allyloxycarbonyl (Alloc), 2-(trimethylsilyl)ethyloxycarbonyl (Teoc), 2,2,2-trichloroethoxycarbonyl (Troc), para-toluenesulfonyl (Tos), 2,2,2-trifluoroacetyl (Tfa), trityl (Trt), 2,4-dimethocybenzyl (Dmb), p-Methoxybenzyl (Pmb), and benzyl (Bn). In some embodiments, R^{EN′} is SEM.

In some embodiments, the method further comprises a deprotecting reaction. In some embodiments, the deprotecting reaction occurs after the condensation reaction, and wherein the deprotecting reaction comprises replacing R^{EN} with a hydrogen. In some embodiments, the deprotecting reaction occurs before the reacting of the compound represented by Formula (E-1) with the compound represented by Formula (F). In some embodiments, the compound represented by Formula (E-1) is deprotected. In some embodiments, the compound of Formula (C), or a pharmaceutically acceptable salt thereof, has a structure of

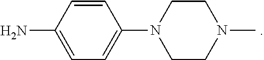

In one aspect, provided herein is a compound represented by Formula (A), or a pharmaceutically acceptable salt thereof,

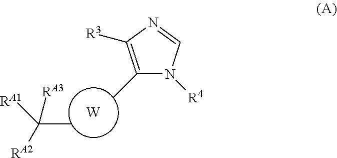

wherein
R³ is selected from optionally substituted C₁-C₆ alkyl, optionally substituted 3- to 10-membered heterocycle and optionally substituted C₃₋₁₀ carbocycle, wherein each of the alkyl, heterocycle and carbocycle is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R⁴ is selected from:
   hydrogen;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
   optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
Ring W is an optionally substituted 5 or 6-membered heteroaryl, wherein the 5 or 6-membered heteroaryl is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
each of R^{A1}, R^{A2} and R^{A3} is independently selected from a halogen and —O—C₁₋₆alkyl.

In some embodiments, the compound of Formula (A) has a structure of

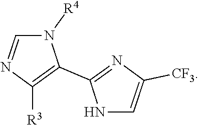

In certain embodiments, the disclosure provides a method of synthesizing a compound represented by Formula (I):

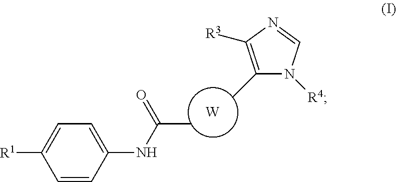

or a pharmaceutically acceptable salt thereof, wherein:
R¹ is selected from:
—N(R⁵)₂, wherein R⁵ is selected from hydrogen, and optionally substituted C₁-C₆ alkyl, wherein the optional substituents on C₁-C₆ alkyl are independently selected at each occurrence from one or more halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
substituted C₁-C₆ alkyl, wherein the substituents on C₁-C₆ alkyl are independently selected at each occurrence from one or more halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, 3- to 12-membered heterocycle;
optionally substituted 3 to 8-membered heterocycle; wherein the optional substituents on the 3 to 8-membered heterocycle are independently selected at each occurrence from one or more halogen, —OH, —CN, —NO₂, —NH₂, oxo, ═S, —S(O₂)NH₂, —C₁₋₁₀ haloalkyl, —O—C₁₋₁₀ alkyl, C₂₋₁₀alkenyl, C₂₋₁₀ alkynyl, C₃₋₁₂ carbocycle, 3- to 12-membered heterocycle, and optionally substituted C₁₋₁₀ alkyl, wherein the optional substituents on the C₁₋₁₀ alkyl are independently selected at each occurrence from one or more hydroxy, halogen, oxo, —C₁₋₁₀haloalkyl, —NH₂, —CN, and —NO₂;
   R³ is selected from optionally substituted C₁-C₆ alkyl, optionally substituted 3- to 10-membered heterocycle and optionally substituted C₃₋₁₀ carbocycle, wherein the optional substituents on each are independently selected at each occurrence from one or more halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
   R⁴ is selected from:
hydrogen;
optionally substituted C₁-C₆ alkyl, wherein the optional substituents on C₁-C₆ alkyl are independently selected at each occurrence from one or more halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
optionally substituted C₃₋₁₀ carbocycle, wherein the optional substituents on C₃- to carbocycle are independently selected at each occurrence from one or more halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle; and Ring W is selected from optionally substituted 5- to 8-membered (e.g., 5 or 6 membered) heteroaryl, wherein the substituents on the optionally substituted 5- to 8-membered heteroaryl are independently selected at each occurrence from one or more halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle.

In certain embodiments, the disclosure provides a method of synthesizing a compound represented by Formula (I):

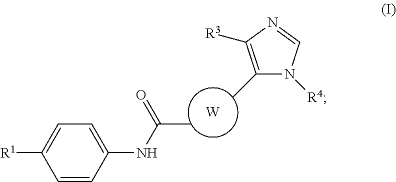

or a pharmaceutically acceptable salt thereof, wherein:
R¹ is selected from:
—N(R⁵)₂, wherein R⁵ is selected from hydrogen and optionally substituted C₁-C₆ alkyl; substituted C₁-C₆ alkyl; and optionally substituted 3 to 14-membered heterocycle (such as 5-6 membered heterocycloalkyl);
   R³ is selected from optionally substituted C₁-C₆ alkyl, optionally substituted 3- to 10-membered heterocycle and optionally substituted C₃₋₁₀ carbocycle;
   R⁴ is selected from:
hydrogen;
optionally substituted C₁-C₆ alkyl or optionally substituted C₁-C₆ heteroalkyl;
optionally substituted C₃₋₁₀ carbocycle or optionally substituted 3- to 12-membered heterocycle; and
   Ring W is selected from optionally substituted 5- to 6-membered heteroaryl.

In some embodiments of Formulas (I), (Ia), (A), (B), (G), (G-1), (H), (H-1), (K) and (J), R⁴ is optionally substituted C₁-C₆ alkyl. In some embodiments, R⁴ is optionally substituted C₃₋₁₀ carbocycle.

In some embodiments, for a compound or salt of Formula (X), (X*) or (I), when R¹ is methylpiperazine and W is pyridine, R⁴ is not methyl. In some cases, when R¹ is

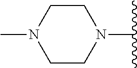 [SMILES: *N1CCN(C)CC1]

and W is pyridine, R⁴ is not methyl.

In some embodiments, for a compound or salt of Formula (X), (X*), or (I), when W is furan, R⁴ is not ethan-1-one. In some cases, when W is furan and R⁴ is cyclopentyl or cyclohexyl, R¹ is not ethan-1-one. In some cases, when W is furan and R⁴ is cyclopentyl or cyclohexyl, R¹ is not ethan-1-one. In some cases, R¹ is not

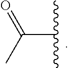 [SMILES: *C(C)=O]

In some embodiments, for a compound or salt of Formula (X), (X*), (A), (B), (E), (G), (G-a), (H), or (I), W is selected from optionally substituted 5- to 6-membered heterocycle. In some cases, the heterocycle of W is a 5- to 6-membered heteroaryl. In some cases, the heterocycle of W is an unsubstituted 5- to 6-membered heteroaryl. In some cases, the heterocycle of W is an unsubstituted 5-membered heteroaryl. In some cases, the heterocycle of W has at least 2 heteroatoms. In some cases, the heterocycle of W has at most 2 heteroatoms. In some cases, the heterocycle of W has only 2 heteroatoms. In some cases, the heterocycle of W is unsubstituted. In some cases, the heterocycle of W has 2 heteroatoms selected from nitrogen, sulfur, and oxygen. In some cases, the heterocycle of W has at least 2 different heteroatoms. In some cases, the heterocycle of W has 2 nitrogen atoms. In some cases, the heterocycle of W has 1 nitrogen atom and I oxygen atom.

In some embodiments, for a compound or salt of Formula (X), (X*), (I), (Ia), (C), or (IIB), R¹ is optionally substituted. In some embodiments, R¹ is optionally substituted with 1 to 4 substituents. In some embodiments, R¹ is optionally substituted with 1 to 3 substituents. In some embodiments, R¹ is optionally substituted with 1 to 2 substituents. In some embodiments, R¹ is optionally substituted with 1 substituent. In some embodiments, R¹ is optionally substituted with 2 substituents. In some embodiments, R¹ is optionally substituted with 3 substituents. In some embodiments, R¹ is monocyclic. In some embodiments, R¹ is bicyclic. In some embodiments, R¹ is a bridged ring. In some embodiments, R¹ is a fused ring. In some embodiments, R¹ is a spiro ring. In some embodiments, R¹ is optionally substituted 3-12 membered ring. In some embodiments, R¹ is optionally substituted 5-8 membered ring.

In some embodiments, for a compound or salt of Formula (X), (X*), (I), (Ia), (C), or (IIB), R¹ is optionally substituted with an oxide.

In some embodiments, for a compound or salt of Formula (X), (X*), (I), (Ia), (C), or (IIB), R¹ is selected from substituted C₁-C₆ alkyl, wherein the substituents on C₁-C₆ alkyl are independently selected at each occurrence from one or more halogen, —OH, —CN, —NO₂, —NH₂, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle. In some embodiments, for a compound or salt of Formula (X), (X*), (I), (Ia), (C), or (IIB), R¹ is selected from substituted C₁-C₆ alkyl, wherein the substituents on C₁-C₆ alkyl are independently selected at each occurrence from one or more halogen, —OH, —CN, —NO₂, —NH₂, ═S, —C₁₋₆ haloalkyl, —O—C₁₋₆ alkyl, —O—C₁₋₆alkyl-O—C(O)(O—C₁₋₆alkyl), C₂₋₆ alkenyl, C₂₋₆ alkynyl, C₃₋₆ carbocycle, and 3- to 12-membered heterocycle. In some embodiments, R¹ is optionally substituted C₁-C₃ alkyl. In some embodiments, R¹ is optionally substituted with one or more substituents selected from oxo, halogen, —O—C₁₋₁₀ alkyl, —C₁₋₁₀ haloalkyl, and —OH.

In some embodiments, for a compound or salt of Formula (X), (X*), (I), (Ia), (C) or (IIB), R¹ is optionally substituted C₁-C₁₀ heteroalkyl. In some embodiments, for a compound or salt of Formula (X), (X*), (I), (Ia), (C), or (IIB), R¹ is optionally substituted C₁-C₆ heteroalkyl wherein the substituents on C₁-C₆ heteroalkyl are independently selected at each occurrence from one or more halogen, —OH, —CN, —NO₂, —NH₂, ═S, —C₁₋₁₀ haloalkyl, —C₂₋₁₀ alkenyl, C₂₋₁₀ alkynyl, C₃₋₁₀ carbocycle, and 3- to 12-membered heterocycle. In some embodiments, for a compound or salt of Formula (X), (X*), (I), (Ia), (C), or (IIB), R¹ is optionally substituted C₁-C₆ heteroalkyl wherein the substituents on C₁-C₆ heteroalkyl are independently selected at each occurrence from one or more halogen, —OH, —CN, —NO₂, —NH₂, ═S, —C₁₋₃ haloalkyl, —C₂₋₆ alkenyl, C₂₋₆ alkynyl, C₃₋₆ carbocycle, and 3- to 12-membered heterocycle.

In some cases, when W is furan, R¹ is selected from substituted C₁-C₆ alkyl, wherein the substituents on C₁-C₆ alkyl are independently selected at each occurrence from one or more halogen, —OH, —CN, —NO₂, —NH₂, oxo, ═S, —C₁₋₁₀haloalkyl, —O—C₁₋₁₀ alkyl, —O—C₁₋₆ alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle.

In some embodiments, for a compound or salt of Formula (X), (X*), (I), (Ia), (C), or (IIB), R¹ is selected from —N(R⁵)₂, In some embodiments, for a compound or salt of Formula (X), (X*), (I), (Ia), (C), or (IIB), R¹ is selected from —N(R⁵)₂, wherein R⁵ is selected from optionally substituted C₁-C₆ alkyl, wherein the optional substituents on C₁-C₆ alkyl are selected from hydroxy.

In some embodiments, for a compound or salt of Formula (X), (X*), (I), (Ia), (C), or (IIB), R¹ is selected from substituted C₁-C₆ alkyl and optionally substituted 3 to 8-membered heterocycle.

In some embodiments, for a compound or salt of Formula (X), (X*), (I), (Ia), (C), or (IIB), R¹ is substituted C₁-C₆ alkyl. In some embodiments, for a compound or salt of Formula (X), (X*), (I), (Ia), (C), or (IIB), R¹ is selected from substituted C₁-C₆ alkyl, wherein the substituents are selected from hydroxy, oxo, and —O—C₁₋₁₀ alkyl.

In some embodiments, for a compound or salt of Formula (X), (X*), (I), (Ia), (C), or (IIB), R¹ is optionally substituted 3 to 8-membered heterocycle. In some embodiments, for a compound or salt of Formula (X), (X*), (I), (Ia), (C), or (IIB), R¹ is selected from optionally substituted 5 to 6-membered heterocycle. In some embodiments, R¹ is monocyclic. In some embodiments, R¹ is bicyclic. In some embodiments, R¹ is a fused bicyclic group. In some embodiments, R¹ is a bridged bicyclic group. In some embodiments, R¹ is optionally substituted 5 membered heterocycle. In some embodiments, R¹ is optionally substituted heteroaryl. In some embodiments, R¹ is optionally substituted heterocycloalkyl. In some embodiments, R¹ contains 0-3 nitrogen and 0-1 oxygen atoms on the ring. In some embodiments, R¹ contains 1-2 nitrogen and 0-1 oxygen atoms on the ring. In some embodiments, R¹ contains 1-2 ring nitrogen atoms. In some embodiments, R¹ contains 2 ring nitrogen atoms. In some embodiments, R¹ contains 1 ring nitrogen atom.

In some embodiments, for a compound or salt of Formula (X), (X*), (I), (Ia), (C), or (IIB), R¹ is an optionally substituted 6-membered heterocycle.

In some embodiments, for a compound or salt of Formula (X), (X*), (I), (Ia), (C), or (IIB), R¹ is an optionally substituted piperazine. In some embodiments, R¹ is an optionally substituted piperazine, wherein the piperazine is attached to the rest of the compound (e.g., attached to the phenyl) via a nitrogen. In some embodiments, for a compound or salt of Formula (X), (X*), (I), (Ia), (C), or (IIB), R¹ is piperazine optionally substituted with one or more ₁₋₆ alkyl. In some embodiments, for a compound or salt of Formula (X), (X*), (I), (Ia), (C), or (IIB), R¹ is piperazine optionally substituted with one or more substituents selected from methyl, ethyl and propyl. In some embodiments, for a compound or salt of Formula (X), (X*), (I), (Ia), (C), or (IIB), R¹ is piperazine optionally substituted with one or more methyl. In some embodiments, for a compound or salt of Formula (X), (X*), (I), (Ia), (C), or (IIB), R¹ is piperazine optionally substituted with one or more ₁₋₆ alkyl, wherein the alkyl is optionally substituted with hydroxy, halogen, oxo, and —NH₂. In some embodiments, for a compound or salt of Formula (X), (X*), (I), (Ia), (C), or (IIB), R¹ is piperazine optionally substituted with an oxide.

In some embodiments, for a compound or salt of Formula (X), (X*), (I), (Ia), (C), or (IIB), the optional substituents on the optionally substituted piperazine of R¹ are selected from oxo, —S(O₂)NH₂, and optionally substituted C₁₋₁₀ alkyl, wherein the optional substituents on the C₁₋₁₀ alkyl are independently selected at each occurrence from one or more hydroxy, halogen, oxo, and —NH₂. In some embodiments, for a compound or salt of Formula (X), (X*), (I), (Ia), (C), or (IIB), the optional substituents on the optionally substituted piperazine of R¹ are selected from oxo, —S(O₂)NH₂, —S(O₂)N(C₁₋₆ alkyl)₂, —S(O₂)NH(C₁₋₆ alkyl), and optionally substituted C₁₋₆ alkyl. In some embodiments, for a compound or salt of Formula (X), (X*), (I), (Ia), (C), or (IIB), R¹ is optionally substituted with a heteroalkyl. In some embodiments, R¹ is optionally substituted with one or more substituents selected from halogen, —CN, —OH, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆alkoxy, C₁-C₆haloalkyl, C₁-C₆hydroxyalkyl, C₁-C₆aminoalkyl, C₁-C₆heteroalkyl, and C₃-C₆cycloalkyl.

In some embodiments, for a compound or salt of Formula (X), (X*), (I), (Ia), (C), or (IIB), R¹ is an optionally substituted 3 to 10-membered heterocycle. In some cases, R¹ is an optionally substituted 4- to 8-membered heterocycle. In some cases, R¹ is an optionally substituted 4-membered heterocycle. In some cases, R¹ is an optionally substituted 6-membered heterocycle. In some cases, when R¹ is piperazine, the piperazine is substituted. In some cases, R¹ is not unsubstituted piperazine. In some cases, R¹ is a substituted 3 to 10-membered heterocycle.

In some embodiments, for a compound or salt of Formula (X), (X*), (I), (Ia), (C), or (IIB), for R¹, the optional substituents of the heterocycle are independently selected at each occurrence from one or more substituents selected from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, and optionally substituted C₁₋₁₀ alkyl, wherein the optional substituents on the C₁₋₁₀ alkyl are independently selected at each occurrence from one or more hydroxy, halogen, oxo, —C₁₋₁₀ haloalkyl, —NH₂, —CN, —O—C₁₋₁₀ alkyl, and —NO₂.

In some embodiments, for a compound or salt of Formula (X), (X*), (I), (Ia), (C), or (IIB), the optional substituents of R¹ are independently selected at each occurrence from one or more substituents selected from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl.

In some embodiments, for a compound or salt of Formula (X), (X*), (I), (Ia), (C), or (IIB), the optional substituents of Rare independently selected at each occurrence from one or more substituents selected from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl.

In some embodiments, for a compound or salt of Formula (X), (X*), (I), (Ia), (C), or (IIB), for R¹, the heterocycle has at least one nitrogen atom, phosphorous atom, or oxygen atom. In some cases, for R¹, the heterocycle has at least one nitrogen atom. In some cases, for R¹, the heterocycle has at least two nitrogen atoms. In some cases, for R¹, the heterocycle has at most two nitrogen atoms. In some cases, for R¹, the heterocycle has at most one nitrogen atom. In some cases, for R¹, the heterocycle has two nitrogen atoms. In some cases, for R¹, the heterocycle is a spiro-heterocycle. In some cases, for R¹, the heterocycle is a bridged heterocycle. In some cases, for R¹, the heterocycle is unsaturated. In some cases, for R¹, the heterocycle is saturated.

In some embodiments, for a compound or salt of Formula (X), (X*), (I), (Ia), (C), or (IIB), R¹ is selected from

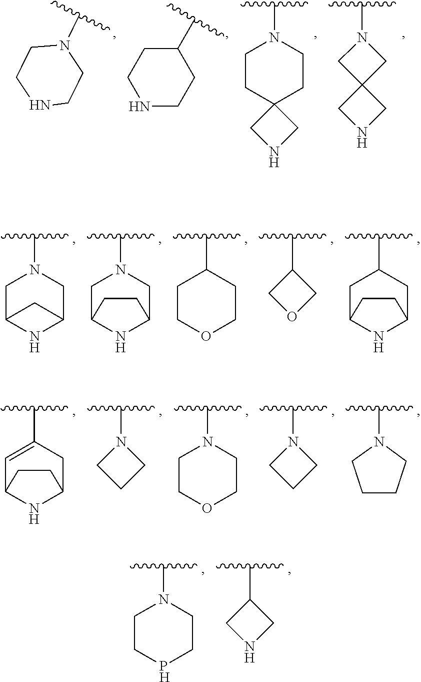

any of which are optionally substituted.

In some embodiments, for a compound or salt of Formula (X), (X*), (I), (Ia), (C), or (IIB), R¹ is selected from

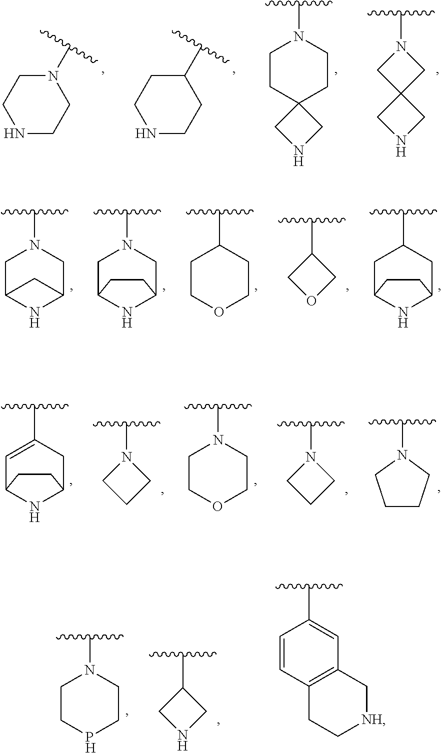

H any of which are optionally substituted with one or more substituents selected from —NH₂, —N(H)C₁-C₆ alkyl, —N(C₁-C₆ alkyl)₂, oxo, and optionally substituted C₁₋₁₀alkyl, wherein the optional substituents on the C₁₋₁₀alkyl are independently selected at each occurrence from one or more oxo and —O—C₁₋₁₀ alkyl. In some embodiments, R¹ is optionally substituted with one or more substituents selected from —NH₂, —N(H)C₁-C₆ alkyl, —N(C₁-C₆ alkyl)₂, oxo, optionally substituted C₁₋₁₀heteroalkyl, and optionally substituted C₁₋₁₀alkyl.

In some embodiments, for a compound or salt of Formula (X), (X*), (I), (Ia), (C), or (IIB), R¹ is selected from

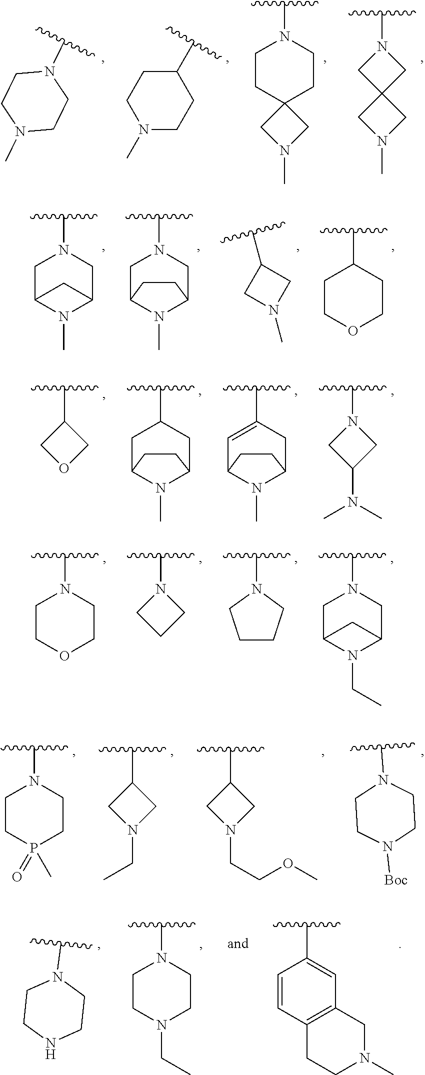

In some embodiments, for a compound or salt of Formula (X), (X*), (I), (Ia), (C), or (IIB), R¹ is selected from

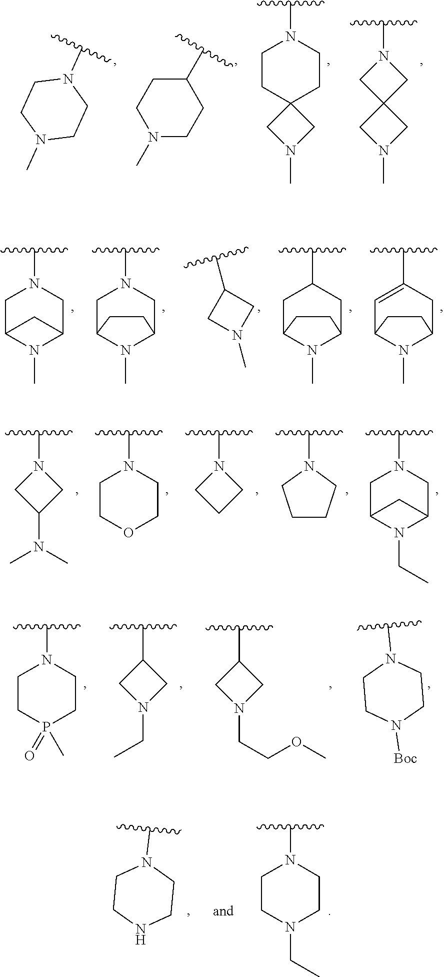

In some embodiments, for a compound or salt of Formula (X), (X*), (I), (Ia), (C), or (IIB), R¹ is selected from

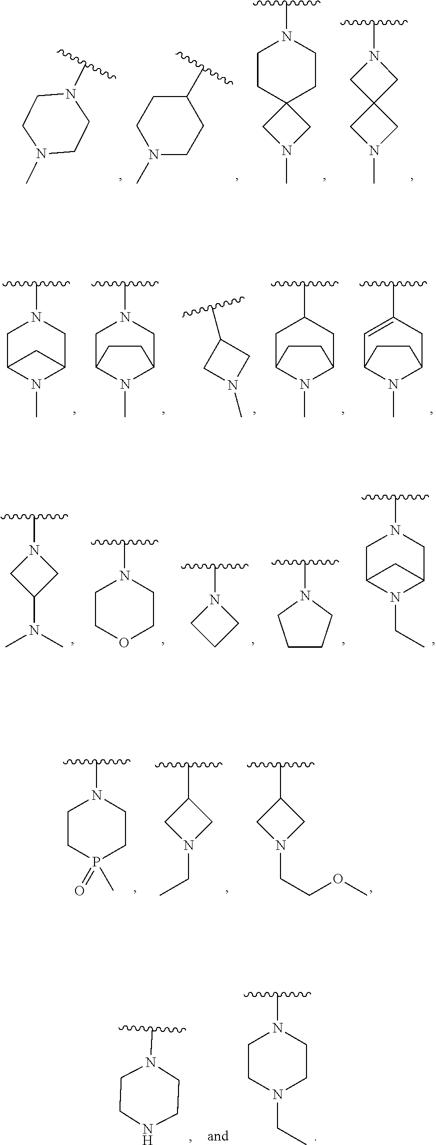

In some embodiments, for a compound or salt of Formula (X), (X*), (I), (Ia), (C), or (IIB), R¹

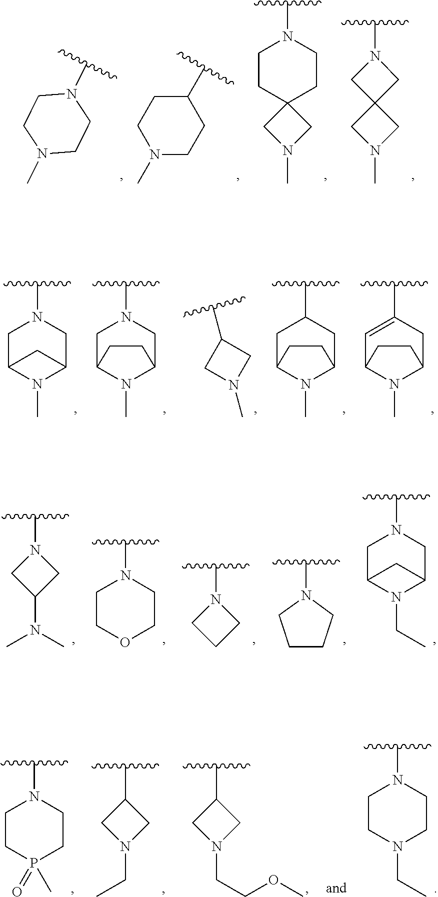

In some embodiments, for a compound or salt of Formula (X), (X*), (I), (Ia), (C), or (IIB), R¹ is selected from

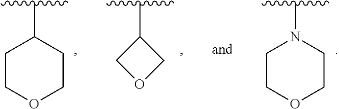 [SMILES: *C1CCOCC1 ; *C1COC1 ; *N1CCOCC1]

In some embodiments, some embodiments, for a compound or salt of Formula (X), (X*), (I), (Ia), (C), or (IIB), R¹ is an optionally substituted 6- to 10-membered heterocycloalkyl. In some cases, the optional substituents of the optionally substituted 6- to 10-membered heterocycloalkyl for R¹ are selected from C₁₋₆ alkyl. In some cases, the 6- to 10-membered heterocycloalkyl is a spiro heterocycloalkyl. In some cases, R¹ is selected from optionally substituted piperazine, optionally substituted diazabicyclo [3.2.1]octane, optionally substituted diazabicyclo[3.1.1]heptane, optionally substituted diazaspiro[3.5]nonane, and optionally substituted diazaspiro[3.3]heptane. In some cases, the optional are selected from C₁₋₆ alkyl.

In some embodiments, for a compound or salt of Formula (X), (X*), (I), (Ia), (A), (B), (F), (F-1), (G), (G-a), (G-1), (G-Ia), (H), (H-1), or (IIB), R³ is optionally substituted. In some embodiments, R³ is optionally substituted with 1 to 4 substituents. In some embodiments, R³ is optionally substituted with 1 to 3 substituents. In some embodiments, R³ is optionally substituted with 1 to 2 substituents. In some embodiments, R³ is optionally substituted with 1 substituent. In some embodiments, R³ is optionally substituted with 2 substituents. In some embodiments, R³ is optionally substituted with 3 substituents.

In some embodiments, for a compound or salt of Formula (X), (X*), (I), (Ia), (A), (B), (F), (F-1), (G), (G-a), (G-1), (G-Ia), (H), (H-1), or (IIB), R³ is selected from optionally substituted C₃₋₆ carbocycle. In some embodiments, for a compound or salt of Formula (X), (X*), (I), (Ia), (A), (B), (F), (F-1), (G), (G-a), (G-1), (G-1a), (H), (H-1), or (IIB), R³ is selected from optionally substituted C₃₋₆ cycloalkyl.

In some embodiments, for a compound or salt of Formula (X), (X*), (I), (Ia), or (IIB), R³ is optionally substituted phenyl. In some embodiments, for a compound or salt of Formula (X), (X*), (I), (Ia), (A), (B), (F), (F-1), (G), (G-a), (G-1), (G-1a), (H), (H-1), or (IIB), R³ is a phenyl optionally substituted with one or more halogen. In some embodiments, for a compound or salt of Formula (X), (X*), (I), (Ia), (A), (B), (F), (F-1), (G), (G-a), (G-1), (G-Ia), (H), (H-1), or (IIB), R³ is a phenyl optionally substituted with 1-3 halogen. In some embodiments, for a compound or salt of Formula (X), (X*), (I), (Ia), (A), (B), (F), (F-1), (G), (G-a), (G-1), (G-1a), (H), (H-1), or (IIB), R³ is a phenyl optionally substituted with 1-2 halogen. In some embodiments, for a compound or salt of Formula (X), (X*), (I), (Ia), (A), (B), (F), (F-1), (G), (G-a), (G-1), (G-1a), (H), (H-1), or (IIB), R³ is a phenyl optionally substituted with one halogen.

In some embodiments, for a compound or salt of Formula (X), (X*), (I), (Ia), (A), (B), (F), (F-1), (G), (G-a), (G-1), (G-Ia), (H), (H-1), or (IIB), the optional substituents of phenyl of R³ are selected from halogen and —C₁₋₁₀ haloalkyl. In some embodiments, for a compound or salt of Formula (X), (X*), (I), (Ia), (A), (B), (F), (F-1), (G), (G-a), (G-1), (G-1a), (H), (H-1), or (IIB), the optional substituents of phenyl of R³ are selected from halogen and —C₁₋₃ haloalkyl.

In some embodiments, for a compound or salt of Formula (X), (X*), (I), (Ia), (A), (B), (G), (G-1), (H), (H-1), (J), (K), or (IIB), R⁴ is unsubstituted. In some embodiments, for a compound or salt of Formula (X), (X*), (I), (Ia), (A), (B), (G), (G-1), (H), (H-1), (J), (K), or (IIB), R⁴ is substituted. In some embodiments, for a compound or salt of Formula (X), (X*), (I), (Ia), (A), (B), (G), (G-1), (H), (H-1), (J), (K), or (IIB), R⁴ is substituted with one or more substituents selected from halogen, —OH, —CN, —NO₂, —NH₂, oxo, ═S, —O—C₁₋₁₀ alkyl, —C₁₋₁₀haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some embodiments, R⁴ is substituted with one or more substituents selected from halogen, —OH, —CN, —NO₂, —NH₂, oxo, ═S, —O—C₁₋₆ alkyl, —C₁₋₆ haloalkyl, —O—C₁-alkyl, C₂₋₆ alkenyl, C₂₋₆ alkynyl, C₃₋₆ carbocycle, and 3- to 12-membered heterocycle. In some embodiments, R⁴ is substituted with one or more substituents selected from halogen, —OH, —CN, —NO₂, —NH₂, oxo, ═S, —O—C₁₋₆ alkyl, —C₁₋₆ haloalkyl, and —O—C₁₋₆ alkyl. In some embodiments, R⁴ is substituted with one or more substituents selected from halogen, —OH, —NO₂, —NH₂, oxo, —C₁₋₆ haloalkyl, and —O—C₁₋₆ alkyl. In some embodiments, R is substituted with one or more halogen. In some embodiments, R⁴ is substituted with 1 halogen. In some embodiments, R⁴ is substituted with 2 halogens. In some embodiments, R⁴ is substituted with 3 halogens.

In some embodiments, for a compound or salt of Formula (X), (X*), (I), (Ia), (A), (B), (G), (G-1), (H), (H-1), (J), (K), or (IIB), R⁴ is hydrogen.

In some embodiments, for a compound or salt of Formula (X), (X*), (I), (Ia), (A), (B), (G), (G-1), (H), (H-1), (J), (K), or (IIB), R is selected from optionally substituted C₁-C₆ alkyl and optionally substituted C₃₋₆ carbocycle. In some embodiments, for a compound or salt of Formula (X), (X*), (I), (Ia), (A), (B), (G), (G-1), (H), (H-1), (J), (K), or (IIB), R⁴ is optionally substituted cycloalkyl. In some embodiments, for a compound or salt of Formula (X), (X*), (I), (Ia), (A), (B), (G), (G-1), (H), (H-1), (J), (K), or (IIB), R⁴ is optionally substituted aryl.

In some embodiments, for a compound or salt of Formula (X), (X*), (I), (Ia), (A), (B), (G), (G-1), (H), (H-1), (J), (K), or (IIB), the optional substituents of C₁-C₆ alkyl of R⁴ are selected from halogen.

In some embodiments, for a compound or salt of Formula (X), (X*), (I), (Ia), (A), (B), (G), (G-1), (H), (H-1), (J), (K), or (IIB), the optional substituents of C₃-C₆ carbocycle of R are selected from hydroxy.

In some embodiments, for a compound or salt of Formula (X), (X*), (A), (B), (E), (G), (G-a), (H), or (I), W is selected from 5- to 6-membered heteroaryl.

In some embodiments, for a compound or salt of Formula (X), (X*), (A), (B), (E), (G), (G-a), (H), or (I), the 5- to 6-membered heteroaryl of W are selected from imidazole, furan, thiophene, oxazole, isoxazole, thiazole, oxadiazole, thiadiazole, pyridine, pyridazine, pyrimidine, and pyrazine.

In some embodiments, for a compound or salt of Formula (X), (X*), (A), (B), (E), (G), (G-a), (H), or (I), the 5- to 6-membered heteroaryl of W are selected from imidazole, furan, and pyridine.

In some embodiments, for a compound or salt of Formula (X), (X*), (A), (B), (E), (G), (G-a), (H), or (I), W is imidazole. In some embodiments, for a compound or salt of Formula (X), (X*), (A), (B), (E), (G), (G-a), (H), or (I), W is pyridine.

In some embodiments, for a compound or salt of Formula (X), (X*), (A), (B), (E), (G), (G-a), (H), or (I), W is selected from optionally substituted 5- to 6-membered heteroaryl.

In some embodiments, for a compound or salt of Formula (X), (X*), (A), (B), (E), (G), (G-a), (H), or (I), W is selected from pyridine, imidazole, thiazole, and furan.

In some embodiments, for a compound or salt of Formula (X), (X*), (A), (B), (E), (G), (G-a), (H), or (I), W is selected from pyridine and imidazole.

In some aspects, the compound or salt of Formula (I) is represented by formula (Ia):

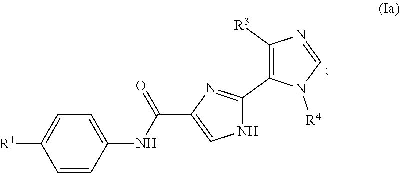

or a pharmaceutically acceptable salt thereof.

In some aspects, the compound or salt of Formula (I) is represented by formula (IIB):

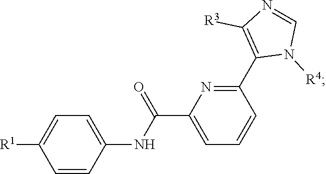

or a pharmaceutically acceptable salt thereof.

In some embodiments, for a compound or salt of Formula (X), (X*), (I), (Ia), (A), (B), (F), (F-1), (G), (G-a), (G-1), (G-1a), (H), (H-1), or (IIB), R³ is selected from optionally substituted C₁-C₆ alkyl and optionally substituted C₆ carbocycle.

In some embodiments, for a compound or salt of Formula (X), (X*), (I), (Ia), (A), (B), (F), (F-1), (G), (G-a), (G-1), (G-1a), (H), (H-1), or (IIB), for a compound or salt of Formula (X), (X*), (I), (Ia), (A), (B), (F), (F-1), (G), (G-a), (G-1), (G-1a), (H), (H-1), or (IIB), R³ is selected from C₁-C₆ alkyl and wherein the C₆ carbocycle is substituted with one or more substituents selected from halogen and —C₁₋₁₀ haloalkyl.

In some embodiments, for a compound or salt of Formula (X), (X*), (I), (Ia), (A), (B), (F), (F-1), (G), (G-a), (G-1), (G-1a), (H), (H-1), or (IIB), R³ is selected from

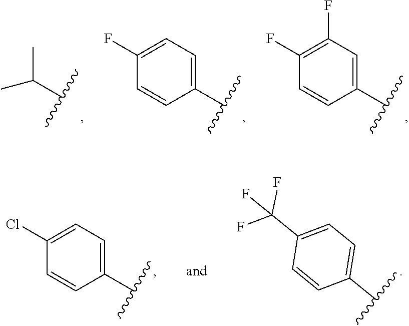

In some embodiments, for a compound or salt of Formula (X), (X*), (I), (Ia), (A), (B), (F), (F-1), (G), (G-a), (G-1), (G-1a), (H), (H-1), or (IIB), R³ is Cₑ carbocycle substituted with one or more substituents selected from halogen.

In some embodiments, for a compound or salt of Formula (X), (X*), (I), (Ia), (A), (B), (F), (F-1), (G), (G-a), (G-1), (G-1a), (H), (H-1), or (IIB), R³ is phenyl, wherein the phenyl is optionally substituted with one or more halogen.

In some embodiments, for a compound or salt of Formula (X), (X*), (I), (Ia), (A), (B), (F), (F-1), (G), (G-a), (G-1), (G-1a), (H), (H-1), or (IIB), R³ is selected from

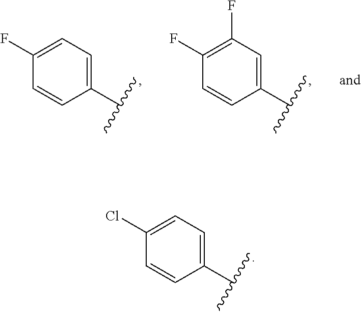 [SMILES: *c1ccc(F)cc1 ; *c1ccc(F)c(F)c1 ; *c1ccc(Cl)cc1]

In some embodiments, for a compound or salt of Formula (X), (X*), (I), (Ia), (A), (B), (F), (F-1), (G), (G-a), (G-1), (G-1a), (H), (H-1), or (IIB), R³ is

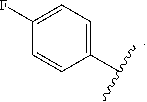 [SMILES: *c1ccc(F)cc1]

In some embodiments, for a compound or salt of Formula (X), (X*), (I), (Ia), (A), (B), (G), (G-1), (H), (H-1), (J), (K), or (IIB), R⁴ is selected from hydrogen, C₁-C₆ alkyl optionally substituted with one or more substituents selected from halogen, and C₅₋₆ carbocycle optionally substituted with one or more substituents selected from hydroxy and amine.

In some embodiments, for a compound or salt of Formula (X), (X*), (I), (Ia), (A), (B), (G), (G-1), (H), (H-1), (J), (K), or (IIB), R⁴ is unsubstituted C₁-C₆ alkyl.

In some embodiments, for a compound or salt of Formula (X), (X*), (I), (Ta), (A), (B), (G), (G-1), (H), (H-1), (J), (K), or (IIB), R⁴ is substituted C₁-C₆ alkyl, wherein the C₁-C₆ alkyl is substituted with one or more halogen.

In some embodiments, for a compound or salt of Formula (X), (X*), (I), (Ia), or (A), (B), (G), (G-1), (H), (H-1), (J), (K), (IIB), R⁴ is selected from

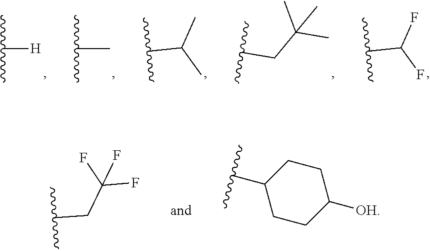

In some embodiments, for a compound or salt of Formula (X), (X*), (I), (Ta), (A), (B), (G), (G-1), (H), (H-1), (J), (K), or (IIB), R⁴ is selected from C₁-C₆ alkyl optionally substituted with one or more substituents selected from fluorine, and C₆ cycloalkyl substituted with hydroxy.

In some embodiments, for a compound or salt of Formula (X), (X*), (I), (Ia), (A), (B), (G), (G-1), (H), (H-1), (J), (K), or (IIB), R⁴ is selected from

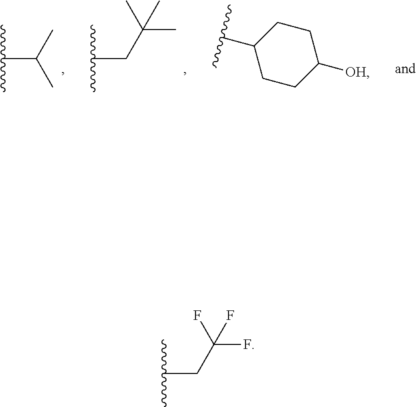

In some embodiments, for a compound or salt of Formula (X), (X*), (I), (Ta), (A), (B), (G), (G-1), (H), (H-1), (J), (K), or (IIB), R⁴ is selected from

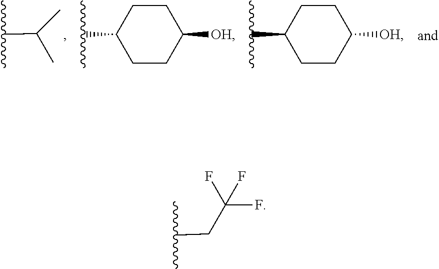

In certain embodiments, R⁴ is

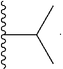 [SMILES: *C(C)C]

In some embodiments, for a compound or salt of Formula (X), (X*), (I), (Ia), (A), (B), (G), (G-1), (H), (H-1), (J), (K), or (IIB), R⁴ is selected from

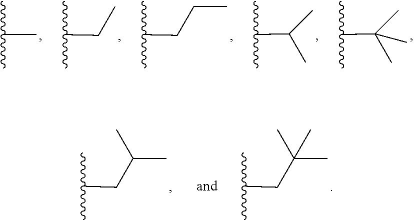

In some embodiments, for a compound or salt of Formula (X), (X*), (I), (Ta), (A), (B), (G), (G-1), (H), (H-1), (J), (K), or (IIB), R⁴ is selected from

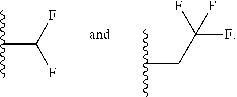

In some cases, R⁴ is selected from unsubstituted C₁₋₁₀ alkyl, unsubstituted 3- to 6-membered heterocycle, and optionally substituted C₃-C₆ carbocycle.

In some embodiments, for a compound or salt of Formula (X), (X*), (I), (Ia), (A), (B), (G), (G-1), (H), (H-1), (J), (K), or (IIB), each R⁴ is selected at each occurrence from C₁₋₁₀ alkyl, C₃₋₁₂ carbocycle, and 3- to 12-membered heterocycle, wherein the C₁₋₁₀ alkyl, C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl.

In some embodiments, for a compound or salt of Formula (X), (X*), (I), (Ia), (A), (B), (G), (G-1), (H), (H-1), (J), (K), or (IIB), R⁴ is selected from unsubstituted C₁₋₁₀ alkyl, unsubstituted 3- to 6-membered heterocycle, and optionally substituted C₃-C₆ carbocycle, wherein the optional substituents are independently selected from one or more halogen —C₁₋₁₀ haloalkyl.

In some embodiments, for a compound or salt of Formula (X), (X*), (I), (Ia), (A), (B), (G), (G-1), (H), (H-1), (J), (K), or (IIB), each R⁴ is selected at each occurrence from C₁₋₁₀ alkyl, unsubstituted 4-membered heterocycle, and optionally substituted C₃-C₅ carbocycle, wherein the optional substituents are independently selected from one or more halogen —C₁₋₁₀ haloalkyl. In some cases, R⁴ is selected from a C₁₋₁₀ alkyl. In some cases, R⁴ is selected from a 4-membered heterocycle. In some cases, R⁴ is a 4-membered heterocycle. In some cases, R⁴ is a saturated 4-membered heterocycle.

In some embodiments, for a compound or salt of Formula (X), (X*), (I), (Ia), (A), (B), (G), (G-1), (H), (H-1), (J), (K), or (IIB), R⁴ is selected from

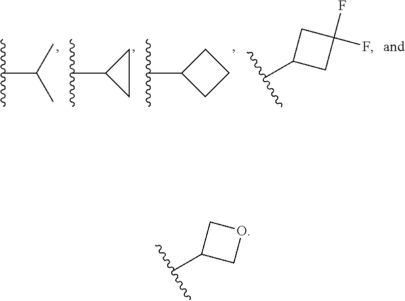

In some cases, R⁴ is selected from

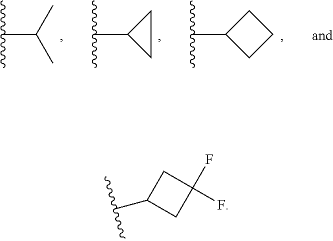

In some cases, R⁴ is selected from

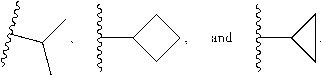

In some embodiments, for a compound or salt of Formula (X), (X*), (I), (Ia), (C), or (IIB), R¹ is selected from:
—N(R⁵)₂, wherein R⁵ is selected from optionally substituted C₁-C₆ alkyl, wherein the substituents on C₁-C₆ alkyl are independently selected at each occurrence from one or more halogen, —OH, —CN, —NO₂, —NH₂, oxo, —C₁₋₁₀haloalkyl, —O—C₁₋₁₀ alkyl,
substituted C₁-C₆ alkyl, wherein the substituents on C₁-C₆ alkyl are independently selected at each occurrence from one or more halogen, —OH, —NH₂, oxo, —C₁₋₁₀haloalkyl, —O—C₁₋₁₀ alkyl, —O—C₁₋₆alkyl-O—C(O)(O—C₁₋₁₀ alkyl);
optionally substituted 6 to 8-membered heterocycle; wherein the optional substituents on the 6 to 8-membered heterocycle are independently selected at each occurrence from one or more oxo, —S(O₂)NH₂, —NH₂, —C₁₋₁₀ haloalkyl, —O—C₁₋₁₀ alkyl, and optionally substituted C₁₋₁₀ alkyl, wherein the optional substituents on the C₁₋₁₀ alkyl are independently selected at each occurrence from one or more hydroxy, halogen, oxo, —C₁₋₁₀ haloalkyl, —NH₂, —CN, and —NO₂.

In some embodiments, for a compound or salt of Formula (X), (X*), (I), (Ia), (C), or (IIB), R¹ is selected from

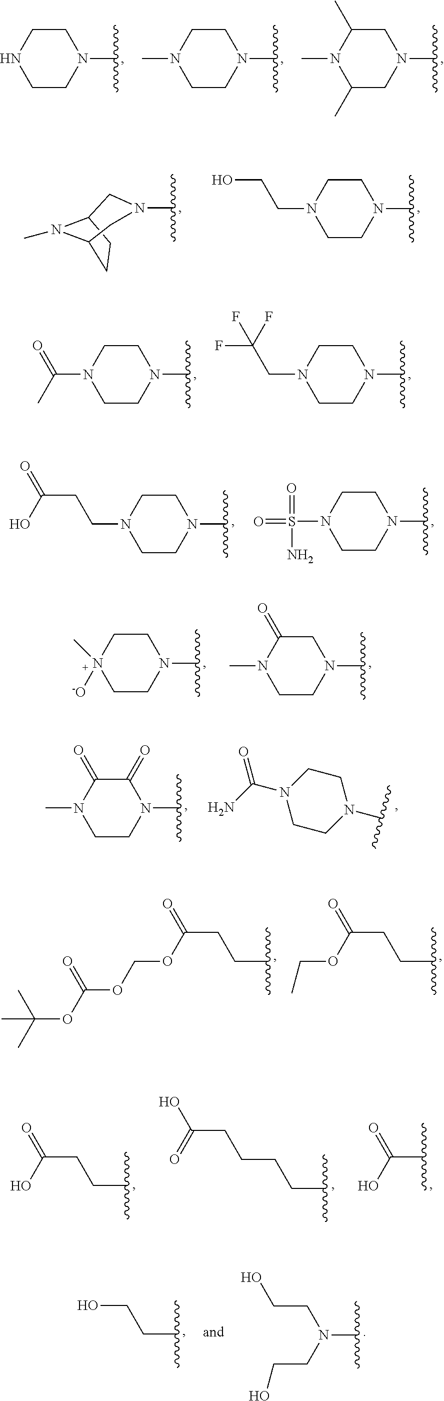

In some embodiments, for a compound or salt of Formula (X), (X*), (I), (Ia), (C), or (IIB), R¹ is selected from

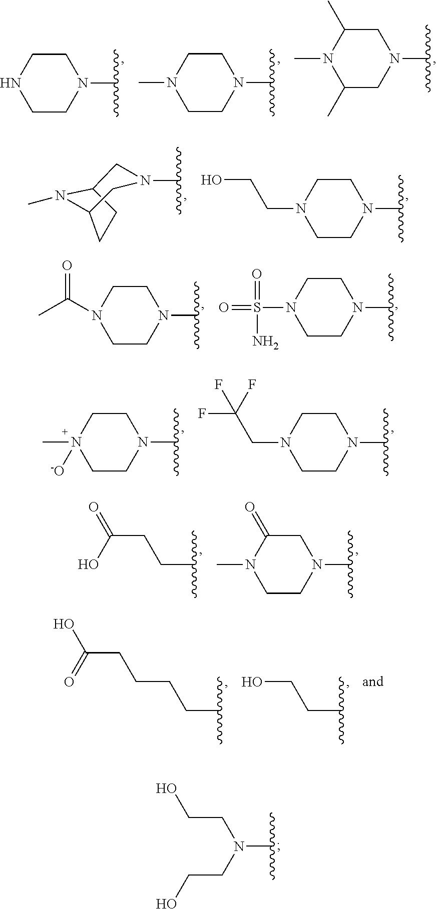

In some embodiments, for a compound or salt of Formula (X), (X*), (I), (Ia), (C), or (IIB), R¹ is selected from:

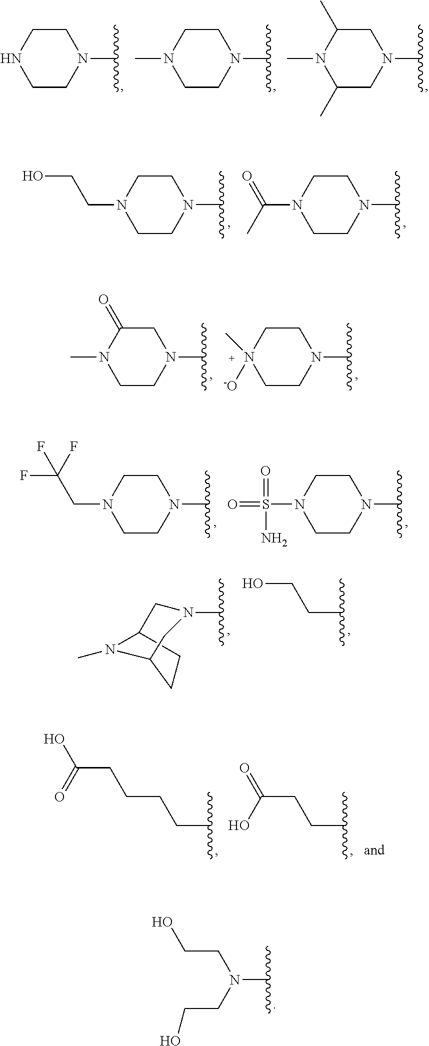

In some embodiments, for a compound or salt of Formula (X), (X*), (I), (Ia), (C), or (IIB), R¹ is selected from:
substituted C₁-C₆ alkyl, wherein the substituents on C₁-C₆ alkyl are independently selected at each occurrence from one or more halogen, —OH, oxo, —C₁₋₁₀ haloalkyl, and —O—C₁₋₁₀ alkyl;
optionally substituted 6 to 8-membered saturated heterocycle; wherein the optional substituents are independently selected at each occurrence from one or more —S(O₂)NH₂, and optionally substituted C₁₋₁₀ alkyl, wherein the optional substituents on the C₁₋₁₀ alkyl are independently selected at each occurrence from one or more hydroxy, halogen, oxo, —C₁₋₁₀ haloalkyl, and —NH₂.

In some embodiments, for a compound or salt of Formula (X), (X*), (I), (Ia), (C), or (IIB), R¹ is selected from

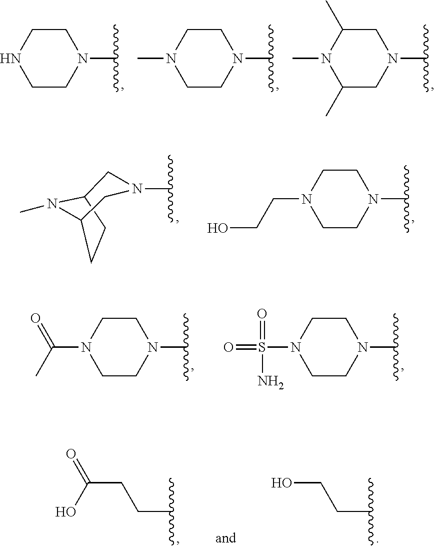

In some embodiments, for a compound or salt of Formula (X), (X*), (I), (Ia), (C), or (IIB), R¹ is selected from optionally substituted 6 to 8-membered saturated heterocycle; wherein the optional substituents are independently selected at each occurrence from one or more —S(O₂)NH₂, and optionally substituted C₁₋₁₀ alkyl, wherein the optional substituents on the C₁₋₁₀ alkyl are independently selected at each occurrence from one or more hydroxy, halogen, oxo, —C₁₋₁₀ haloalkyl, and —NH₂.

In some embodiments, for a compound or salt of Formula (X), (X*), (I), (Ia), (C), or (IIB), R¹ is selected from

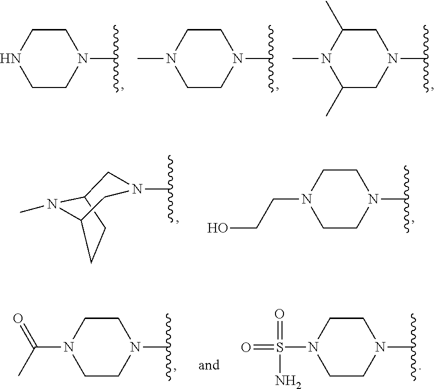

In some embodiments, for a compound or salt of Formula (X), (X*), (I), (Ia), (C), or (IIB), R¹ is selected from substituted C₁-C₆ alkyl, wherein the substituents on C₁-C₆ alkyl are independently selected at each occurrence from one or more halogen, —OH, oxo, —C₁₋₁₀ haloalkyl, and —O—C₁₋₁₀ alkyl.

In some embodiments, for a compound or salt of Formula (X), (X*), (I), (Ia), (C), or (IIB), R¹ is selected from

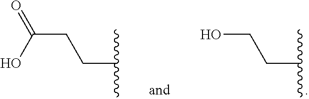

In some embodiments, for a compound or salt of Formula (X), (X*), (I), (Ia), (C), or (IIB), R¹ is selected from:
substituted C₁-C₆ alkyl, wherein the substituents on C₁-C₆ alkyl are independently selected at each occurrence from one or more —OH, oxo, and —O—C₁₋₁₀ alkyl;
optionally substituted 6 to 8-membered saturated heterocycle; wherein the optional substituents are independently selected at each occurrence from one or more optionally substituted C₁₋₁₀ alkyl, wherein the optional substituents on the C₁₋₁₀ alkyl are independently selected at each occurrence from one or more hydroxy, oxo, —C₁₋₁₀ haloalkyl, and —NH₂.

In some embodiments, for a compound or salt of Formula (X), (X*), (I), (Ta), (C), or (IIB), R¹ is selected from:

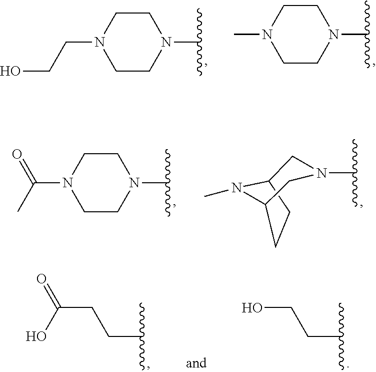

In some embodiments, for a compound or salt of Formula (X), (X*), (I), (Ia), (C), or (IIB), R¹ is piperazine, wherein the piperazine is optionally substituted with one or more substituents independently selected at each occurrence from oxo, —S(O₂)NH₂, and C₁₋₁₀ alkyl, wherein the C₁₋₁₀ alkyl is optionally substituted with one or more substituents independently selected at each occurrence from hydroxy, halogen, oxo, and —NH₂. In some embodiments, R¹ is piperazine substituted with one or more C₁₋₃ alkyl, wherein the C₁₋₃ alkyl is optionally substituted with one or more substituents independently selected at each occurrence from hydroxy, halogen, oxo, and —NH₂.

In some embodiments, for a compound or salt of Formula (X), (X*), (I), (Ia), (C), or (IIB), R¹ is selected from:

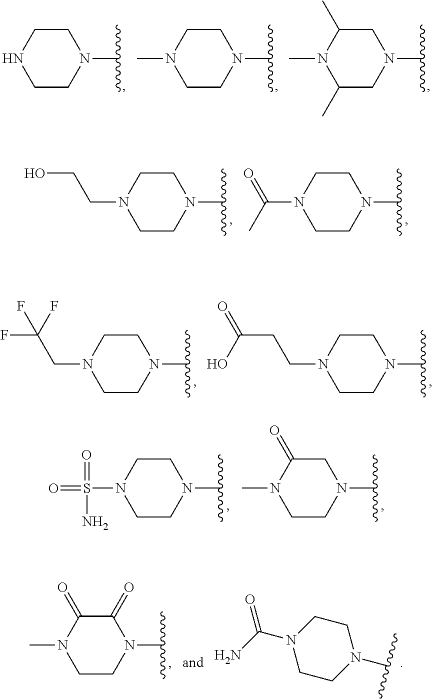

In some embodiments, for a compound or salt of Formula (X), (X*), (C), or (I), the compound is not

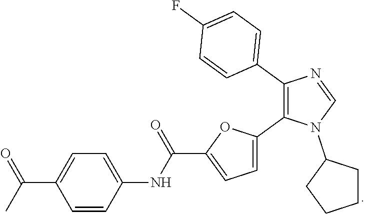 [SMILES: CC(=O)c1ccc(NC(=O)c2ccc(-c3c(-c4ccc(F)cc4)ncn3C3CCCC3)o2)cc1]

In some cases, the compound is not

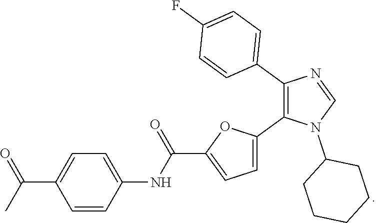 [SMILES: CC(=O)c1ccc(NC(=O)c2ccc(-c3c(-c4ccc(F)cc4)ncn3C3CCCCC3)o2)cc1]

In some embodiments, for a compound or salt of Formula (I),
R¹ is selected from —N(R⁵)₂, wherein R⁵ is selected from optionally substituted C₁-C₆ alkyl, wherein the optional substituents on C₁-C₆ alkyl are selected from hydroxy;
substituted C₁-C₆ alkyl wherein the substituents are selected from hydroxy, oxo, and —O—C₁₋₁₀ alkyl; and
optionally substituted 5 to 6-membered heterocycle wherein the optional substituents are selected from oxo, —S(O₂)NH₂, and optionally substituted C₁₋₁₀ alkyl, wherein the optional substituents on the C₁₋₁₀ alkyl are independently selected at each occurrence from one or more hydroxy, halogen, oxo, and —NH₂;
R³ is optionally substituted phenyl wherein the optional substituents of phenyl of R³ are selected from halogen and —C₁₋₁₀ haloalkyl;
R⁴ is selected from optionally substituted C₁-C₆ alkyl and optionally substituted C₃₋₆ carbocycle wherein the optional substituents of C₁-C₆ alkyl of R⁴ are selected from halogen and wherein the optional substituents of C₃-C₆ carbocycle of R⁴ are selected from hydroxy; and
W is selected from imidazole, furan, and pyridine.

In some embodiments, for a compound or salt of Formula (I),
R¹ is selected from —N(R⁵)₂, wherein R⁵ is selected from optionally substituted C₁-C₆ alkyl, wherein the optional substituents on C₁-C₆ alkyl are selected from hydroxy;
substituted C₁-C₆ alkyl wherein the substituents are selected from hydroxy, oxo, and —O—C₁₋₁₀ alkyl; and
optionally substituted 5 to 6-membered heterocycle wherein the optional substituents are selected from oxo, —S(O₂)NH₂, and optionally substituted C₁₋₁₀ alkyl, wherein the optional substituents on the C₁₋₁₀, alkyl are independently selected at each occurrence from one or more hydroxy, halogen, oxo, and —NH₂; R³ is optionally substituted phenyl wherein the optional substituents of phenyl of R³ are selected from halogen and —C₁₋₁₀ haloalkyl;
R⁴ is selected from optionally substituted C₁-C₆ alkyl and optionally substituted C₃₋₆ carbocycle wherein the optional substituents of C₁-C₆ alkyl of R⁴ are selected from halogen and wherein the optional substituents of C₃-C₆ carbocycle of R⁴ are selected from hydroxy; and
W are selected from imidazole.

In some embodiments, for a compound or salt of Formula (X), (X*), (Ta), (IIB), or (I), R¹ is selected from

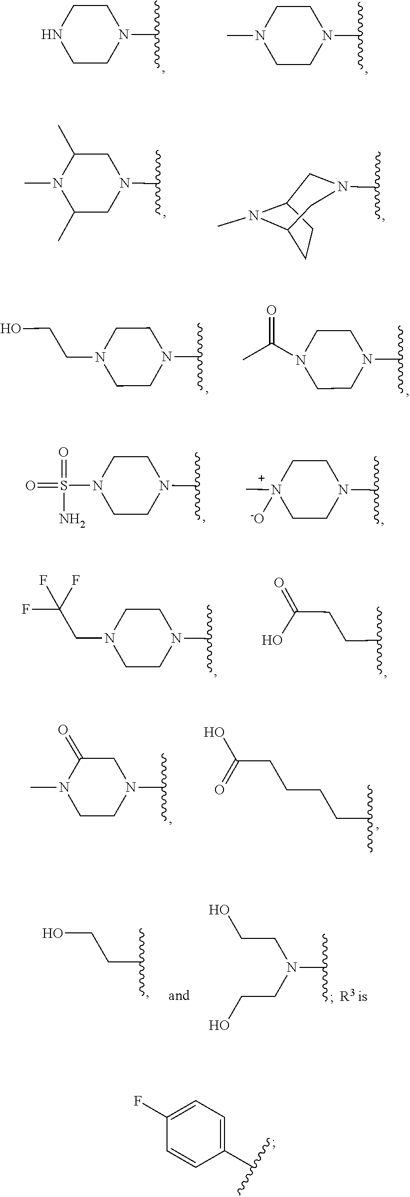

and R₄ is selected from

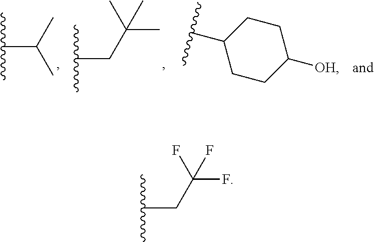

In one aspect, provided herein is a method of synthesizing a compound represented by Formula (Ia):

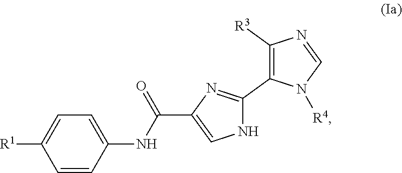

or a pharmaceutically acceptable salt thereof, wherein:
R¹ is piperazine, wherein the piperazine is optionally substituted with one or more substituents independently selected at each occurrence from oxo, —S(O₂)NH₂, and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is unsubstituted C₁-C₆ alkyl.

In some embodiments of Formulas (I), (A), (B), (Ia), (K), (G), (H), (G-a), (J), (G-1), (H-1) and (K), R⁴ is selected from

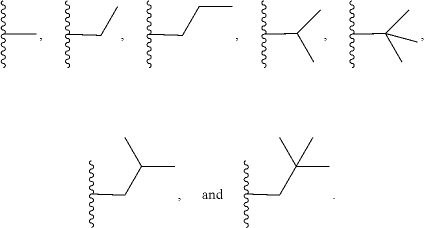

In some embodiments of Formulas (I), (A), (B), (Ia), (F), (G), (H), (G-a), (G), (H), (G-1a), (G-1), and (H-1), R³ is selected from

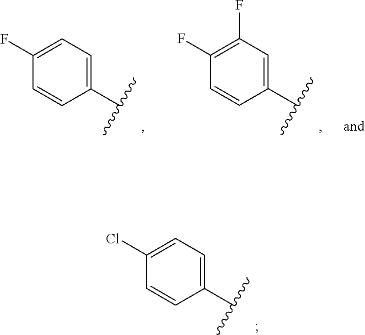 [SMILES: *c1ccc(F)cc1 ; *c1ccc(F)c(F)c1 ; *c1ccc(Cl)cc1]

In some embodiments of Formulas (I), (Ia), and (C), R¹ is piperazine substituted with one or more C₁₋₃ alkyl, wherein the C₁₋₃ alkyl is optionally substituted with one or more substituents independently selected at each occurrence from hydroxy, halogen, oxo, and —NH₂.

In some embodiments of Formulas (I), (Ia), and (C), R¹ is selected from

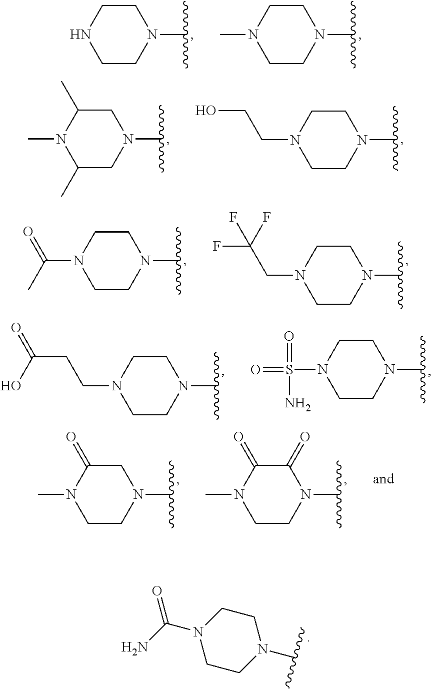

In some embodiments of Formulas (I), (Ia), and (C),
R¹ is selected from

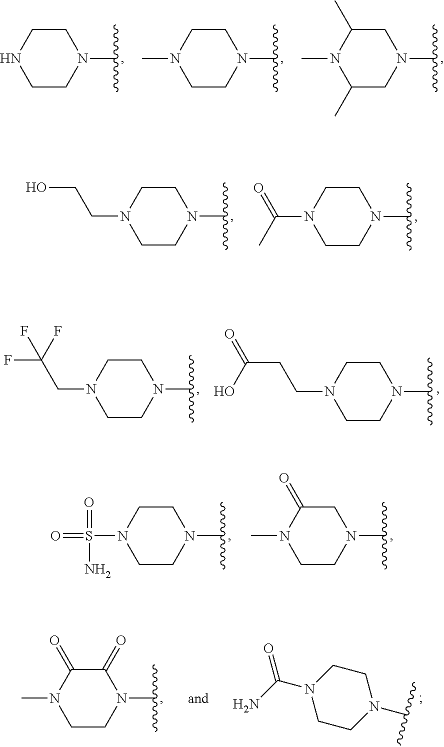

R³ is selected from

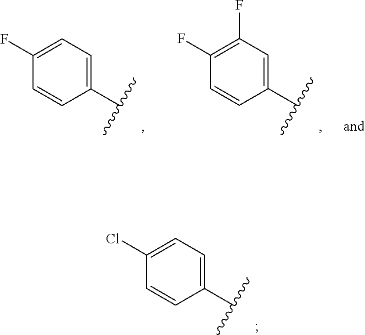 [SMILES: *c1ccc(F)cc1 ; *c1ccc(F)c(F)c1 ; *c1ccc(Cl)cc1]

and
R⁴ is selected from

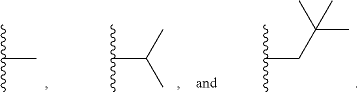

In some embodiments, provided herein is a method of synthesizing a compound, wherein the compound is

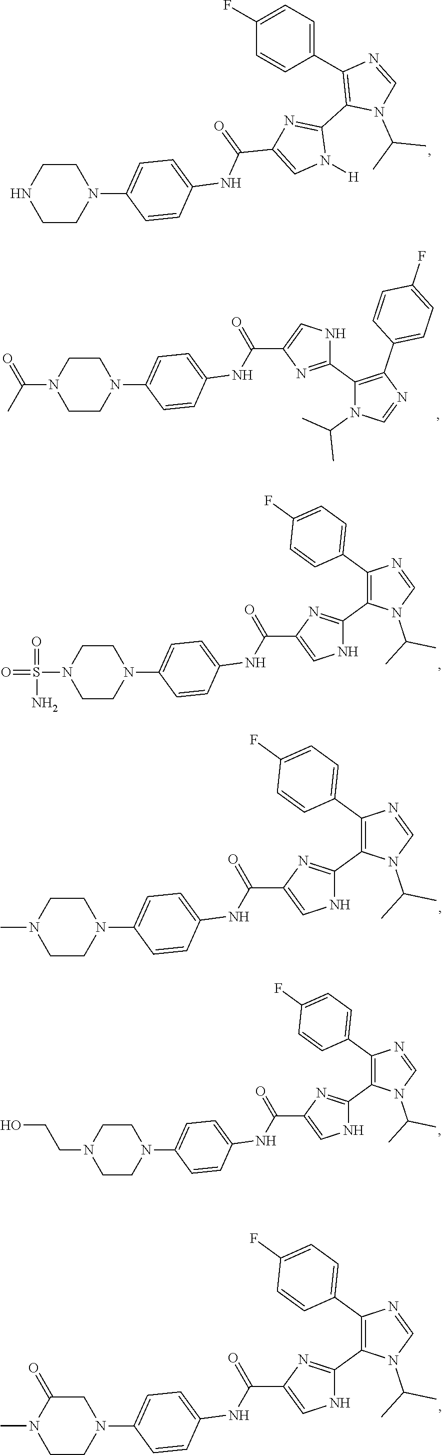

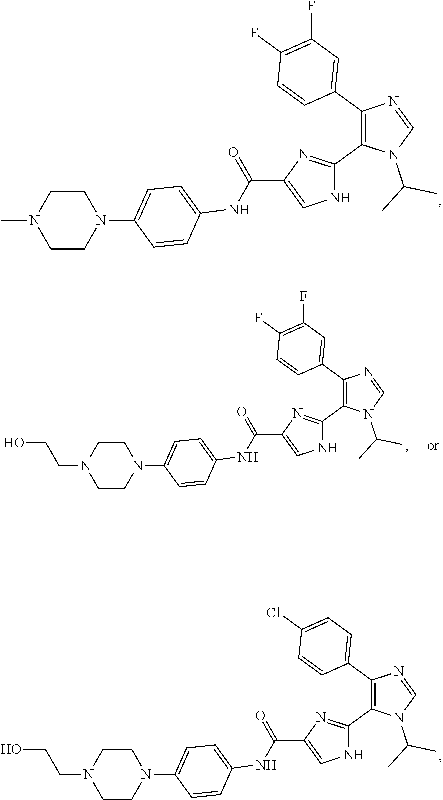 [SMILES: CC(C)n1cnc(-c2ccc(F)c(F)c2)c1-c1nc(C(=O)Nc2ccc(N3CCN(C)CC3)cc2)c[nH]1 ; CC(C)n1cnc(-c2ccc(F)c(F)c2)c1-c1nc(C(=O)Nc2ccc(N3CCN(CCO)CC3)cc2)c[nH]1 ; CC(C)n1cnc(-c2ccc(Cl)cc2)c1-c1nc(C(=O)Nc2ccc(N3CCN(CCO)CC3)cc2)c[nH]1]

or a pharmaceutically acceptable salt thereof. In some embodiments, the compound is

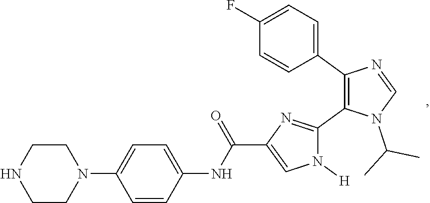 [SMILES: CC(C)n1cnc(-c2ccc(F)cc2)c1-c1nc(C(=O)Nc2ccc(N3CCNCC3)cc2)c[nH]1]

or a pharmaceutically acceptable salt thereof. In some embodiments, the compound is

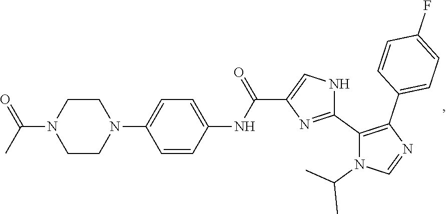 [SMILES: CC(=O)N1CCN(c2ccc(NC(=O)c3c[nH]c(-c4c(-c5ccc(F)cc5)ncn4C(C)C)n3)cc2)CC1]

or a pharmaceutically acceptable salt thereof. In some embodiments, the compound is

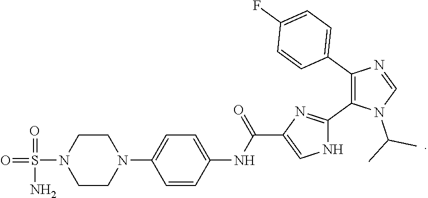 [SMILES: CC(C)n1cnc(-c2ccc(F)cc2)c1-c1nc(C(=O)Nc2ccc(N3CCN(S(N)(=O)=O)CC3)cc2)c[nH]1]

or a pharmaceutically acceptable salt thereof. In some embodiments, the compound is

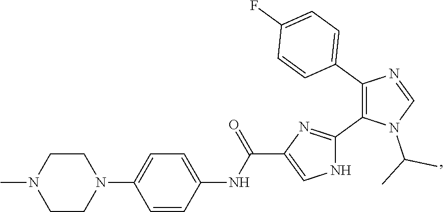 [SMILES: CC(C)n1cnc(-c2ccc(F)cc2)c1-c1nc(C(=O)Nc2ccc(N3CCN(C)CC3)cc2)c[nH]1]

or a pharmaceutically acceptable salt thereof. In some embodiments, the compound is

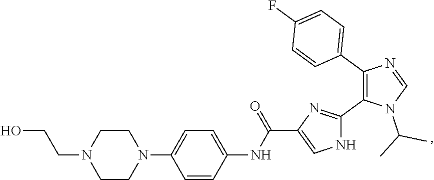 [SMILES: CC(C)n1cnc(-c2ccc(F)cc2)c1-c1nc(C(=O)Nc2ccc(N3CCN(CCO)CC3)cc2)c[nH]1]

or a pharmaceutically acceptable salt thereof. In some embodiments, the compound is

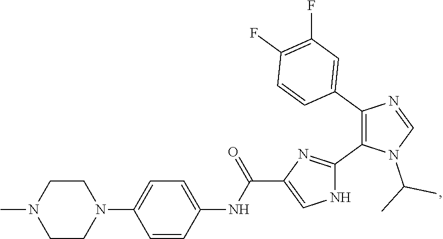 [SMILES: CC(C)n1cnc(-c2ccc(F)c(F)c2)c1-c1nc(C(=O)Nc2ccc(N3CCN(C)CC3)cc2)c[nH]1]

or a pharmaceutically acceptable salt thereof. In some embodiments, the compound is

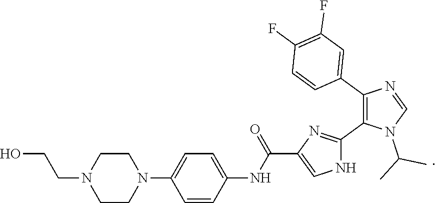 [SMILES: CC(C)n1cnc(-c2ccc(F)c(F)c2)c1-c1nc(C(=O)Nc2ccc(N3CCN(CCO)CC3)cc2)c[nH]1]

or a pharmaceutically acceptable salt thereof. In some embodiments, the compound is

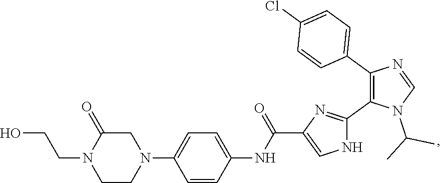 [SMILES: CC(C)n1cnc(-c2ccc(Cl)cc2)c1-c1nc(C(=O)Nc2ccc(N3CCN(CCO)C(=O)C3)cc2)c[nH]1]

or a pharmaceutically acceptable salt thereof. In some embodiments, the compound is

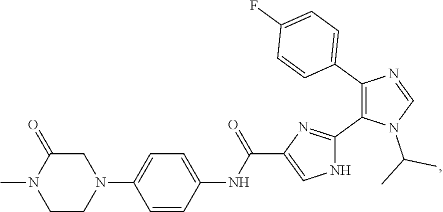 [SMILES: CC(C)n1cnc(-c2ccc(F)cc2)c1-c1nc(C(=O)Nc2ccc(N3CCN(C)C(=O)C3)cc2)c[nH]1]

or a pharmaceutically acceptable salt thereof.

In one aspect, provided herein is a method of synthesizing a compound represented by Formula (Ia):

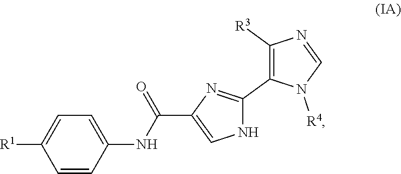

or a pharmaceutically acceptable salt thereof, wherein:
R¹ is piperazine, wherein the piperazine is optionally substituted with one or more substituents independently selected at each occurrence from oxo and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is substituted C₁-C₆ alkyl, wherein the C₁-C₆ alkyl is substituted with one or more halogen.

In some embodiments of Formulas (I), (A), (B), (Ia), (K), (G), (H), (G-a), (J), (G-1), (H-1) and (K), R⁴ is substituted with two or three fluorine.

In some embodiments of Formulas I A), (B), (Ia), (K), (G), (H), (G-a), (J), (G-1), (H-1) and (K), R⁴ is selected from

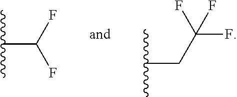

In some embodiments of Formulas (I), (A), (B), (Ia), (F), (G), (H), (G-a), (G), (H), (G-1a), (G-1), and (H-1), R³ is selected from

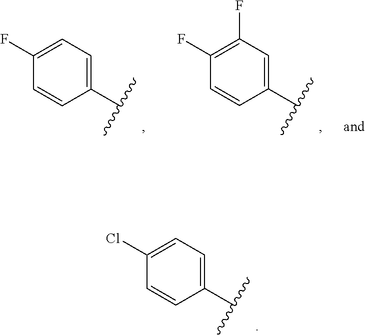 [SMILES: *c1ccc(F)cc1 ; *c1ccc(F)c(F)c1 ; *c1ccc(Cl)cc1]

In some embodiments of Formulas (I), (Ia), and (C), R¹ is piperazine substituted with one or more C₁₋₃ alkyl, wherein the C₁₋₃ alkyl is optionally substituted with one or more halogen.

In some embodiments of Formulas (I), (Ia), and (C), R¹ is selected from

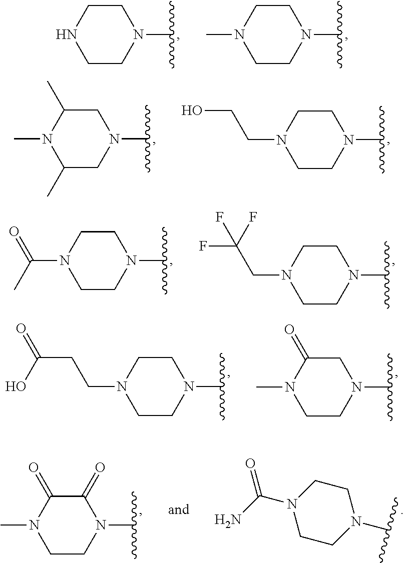

In some embodiments, R¹ is selected from

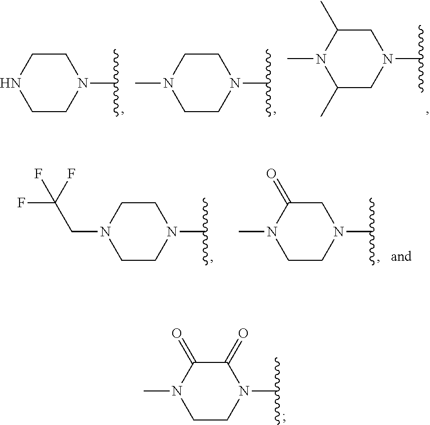

In some embodiments, R¹ is selected from

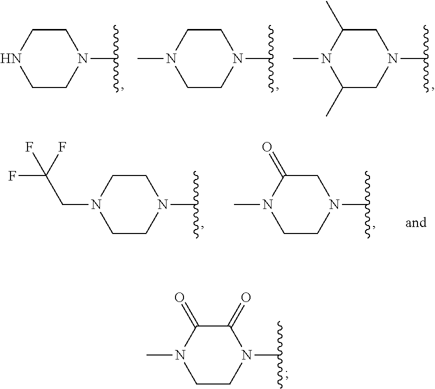

R³ is selected from

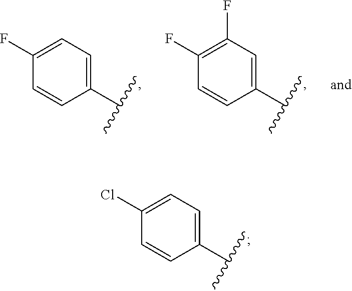 [SMILES: *c1ccc(F)cc1 ; *c1ccc(F)c(F)c1 ; *c1ccc(Cl)cc1]

and
R⁴ is selected from

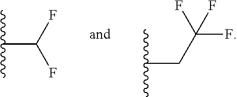

In some embodiments, provided herein is a method of synthesizing a compound, wherein the compound is selected from:

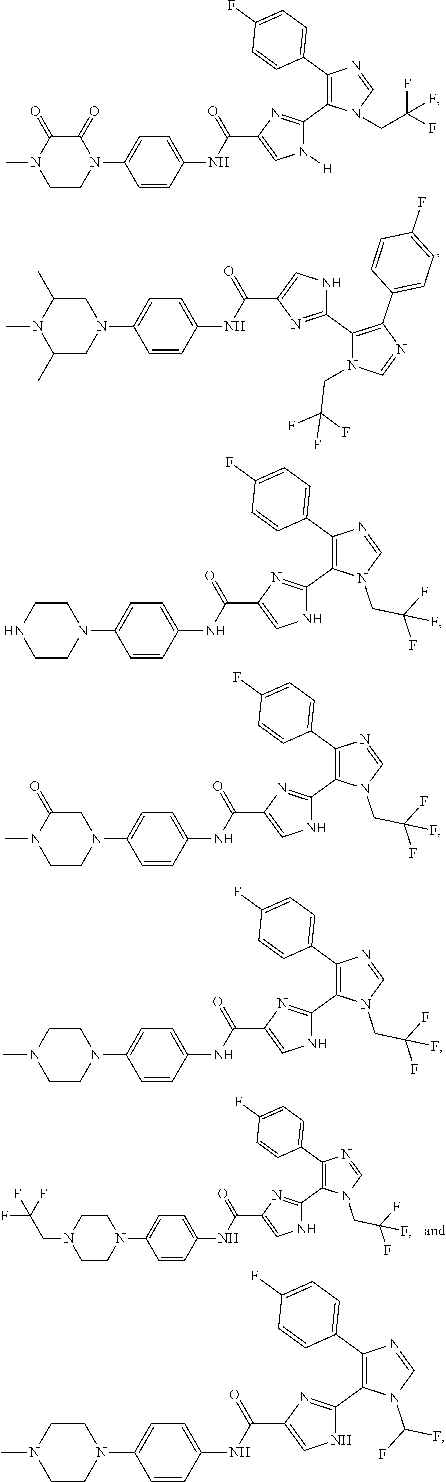

or a pharmaceutically acceptable salt thereof. In some embodiments, the compound is

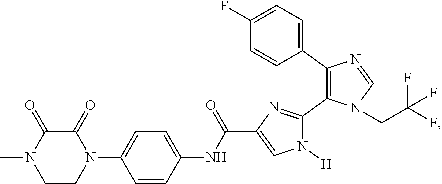 [SMILES: CN1CCN(c2ccc(NC(=O)c3c[nH]c(-c4c(-c5ccc(F)cc5)ncn4CC(F)(F)F)n3)cc2)C(=O)C1=O]

or a pharmaceutically acceptable salt thereof. In some embodiments, the compound is

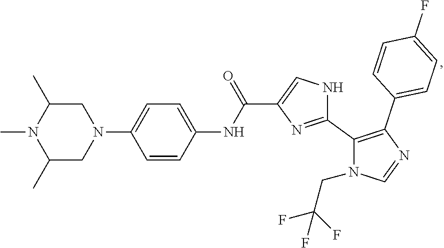 [SMILES: CC1CN(c2ccc(NC(=O)c3c[nH]c(-c4c(-c5ccc(F)cc5)ncn4CC(F)(F)F)n3)cc2)CC(C)N1C]

or a pharmaceutically acceptable salt thereof. In some embodiments, the compound is

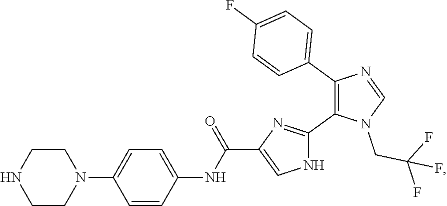 [SMILES: O=C(Nc1ccc(N2CCNCC2)cc1)c1c[nH]c(-c2c(-c3ccc(F)cc3)ncn2CC(F)(F)F)n1]

or a pharmaceutically acceptable salt thereof. In some embodiments, the compound is

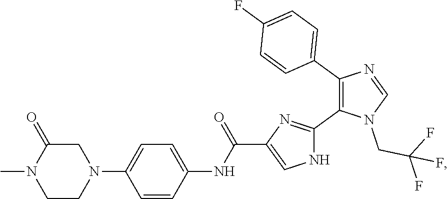 [SMILES: CN1CCN(c2ccc(NC(=O)c3c[nH]c(-c4c(-c5ccc(F)cc5)ncn4CC(F)(F)F)n3)cc2)CC1=O]

or a pharmaceutically acceptable salt thereof. In some embodiments, the compound is

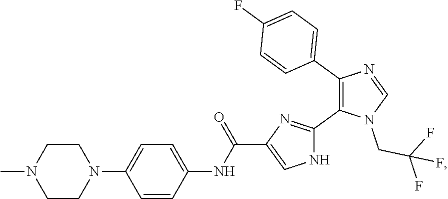 [SMILES: CN1CCN(c2ccc(NC(=O)c3c[nH]c(-c4c(-c5ccc(F)cc5)ncn4CC(F)(F)F)n3)cc2)CC1]

or a pharmaceutically acceptable salt thereof. In some embodiments, the compound is

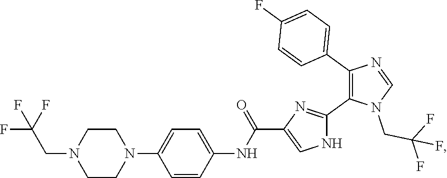 [SMILES: O=C(Nc1ccc(N2CCN(CC(F)(F)F)CC2)cc1)c1c[nH]c(-c2c(-c3ccc(F)cc3)ncn2CC(F)(F)F)n1]

or a pharmaceutically acceptable salt thereof.

In certain embodiments, the disclosure provides a method of synthesizing a compound represented by Formula (X):

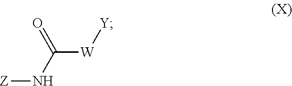

or a pharmaceutically acceptable salt thereof, wherein:
Z is selected from optionally substituted 3- to 12-membered heterocycle and optionally substituted C₃-C₁₂ carbocycle, wherein the substituents on each are independently selected at each occurrence from one or more —N(R¹⁰)₂, halogen, —OH, —CN, —NO₂, —NH₂, oxo, ═S, —C₁₋₁₀haloalkyl, —O—C₁₋₁₀ alkyl, C₂₋₁₀ alkenyl, C₂₋₁₀ alkynyl, C₁-C₁₀ alkyl, C₃₋₁₂ carbocycle, 3- to 12-membered heterocycle;
   wherein the C₁-C₁₀ alkyl is optionally substituted with one or more substituents independently selected at each occurrence from halogen, —OH, —CN, —NO₂, —NH₂, oxo, ═S, —C₁₋₁₀haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, 3- to 12-membered heterocycle;
   wherein the C₃₋₁₂ carbocycle and 3- to 12-membered heterocycle are each optionally substituted at each occurrence from one or more substituents independently selected from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the optional substituents on the C₁₋₁₀ alkyl are independently selected at each occurrence from one or more hydroxy, halogen, oxo, —C₁₋₁₀ haloalkyl, —NH₂, —CN, and —NO₂:
R¹⁰ is selected from optionally substituted C₁-C₆ alkyl, wherein the optional substituents on C₁-C₆ alkyl are independently selected at each occurrence from one or more halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
W is selected from optionally substituted 5- to 8-membered (e.g., 5 or 6 membered) heterocycle and optionally substituted C₃-C₈ carbocycle, wherein the substituents on each are independently selected at each occurrence from one or more halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, wherein the C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl; and
Y is selected from optionally substituted 5- to 8-membered heterocycle and optionally substituted C₃-C₈ carbocycle, wherein the substituents on each are independently selected at each occurrence from one or more halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, wherein the C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl.

In some aspects, the compound of Formula (X) is represented by Formula (X*).

In some embodiments, for a compound or salt of Formula (X), Z is selected from optionally substituted 3- to 12-membered heterocycle and optionally substituted C₃-C₁₂ carbocycle, wherein the substituents on each are independently selected at each occurrence from one or more halogen, —OH, —CN, —NO₂, —NH₂, oxo, ═S, —C₁₋₁₀ haloalkyl, —O—C₁₋₁₀ alkyl. In some cases, for Z, the heterocycle includes at least one nitrogen atom. In some cases, Z is selected from optionally substituted phenyl and optionally substituted pyridine. In some cases, the optional substituents of the optionally substituted phenyl of Z are selected from one or more substituents selected from halogen, —OH, —CN, —NO₂, —NH₂, oxo, ═S, C₁₋₁₀ alkyl, —C₁₋₁₀ haloalkyl, and —O—C₁₋₁₀ alkyl. In some cases, the optional substituents of the optionally substituted phenyl of Z are selected from one or more substituents selected from halogen and C₁₋₁₀ alkyl. In some cases, the heterocycle is unsubstituted. In some cases, Z is selected from substituted phenyl and unsubstituted pyridine. In some cases, the heterocycle has 1 or 2 nitrogen atoms. In some cases, the heterocycle has only 1 nitrogen atom. In some cases, the heterocycle has only 2 nitrogen atoms. In some cases, the heterocycle is a 6-membered heterocycle. In some cases, Z is selected from

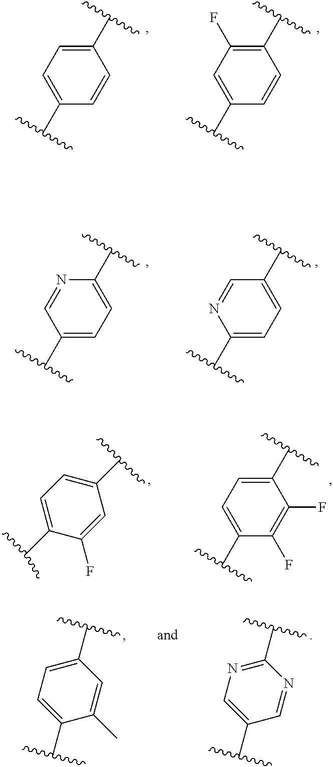

In some cases, the optional substituents of the optionally substituted phenyl of Z is halogen. In some cases, Z is selected from

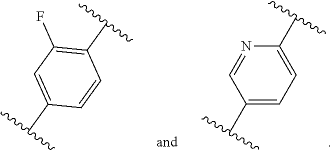 [SMILES: *c1ccc(*)c(F)c1 ; *c1ccc(*)nc1]

In some cases, Z is substituted phenyl. In some cases, Z is phenyl substituted with halogen.

In some aspects, the disclosure provides a method of synthesizing a compound represented by Formula (X*):

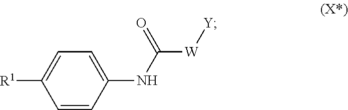

R¹ is selected from:
   —N(R⁵)₂, wherein R⁵ is selected from hydrogen, and optionally substituted C₁-C₆ alkyl, wherein the optional substituents on C₁-C₆ alkyl are independently selected at each occurrence from one or more halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
   optionally substituted C₁-C₆ alkyl, wherein the substituents on C₁-C₆ alkyl are independently selected at each occurrence from one or more halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, 3- to 12-membered heterocycle;
   optionally substituted 3 to 8-membered heterocycle; wherein the optional substituents on the 3 to 8-membered heterocycle are independently selected at each occurrence from one or more halogen, —OH, —CN, —NO₂, —NH₂, oxo, ═S, —S(O₂)NH₂, —C₁₋₁₀haloalkyl, —O—C₁₋₁₀alkyl, C₂₋₁₀alkenyl, C₂₋₁₀ alkynyl, C₃₋₁₂ carbocycle, 3- to 12-membered heterocycle, and optionally substituted C₁₋₁₀ alkyl, wherein the optional substituents on the C₁₋₁₀ alkyl are independently selected at each occurrence from one or more hydroxy, halogen, oxo, —C₁₋₁₀ haloalkyl, —NH₂, —CN, and —NO₂;
W is selected from optionally substituted 5- to 8-membered heterocycle and optionally substituted C₃-C₈ carbocycle, wherein the substituents on each are independently selected at each occurrence from one or more halogen, —OH, —CN, —NO₂, —NH₂, oxo, ═S, —C₁₋₁₀haloalkyl, —O—C₁₋₁₀ alkyl, C₂₋₁₀ alkenyl, C₂₋₁₀ alkynyl, C₃₋₁₂ carbocycle, 3- to 12-membered heterocycle, wherein the C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl; and
Y is selected from optionally substituted 5- to 8-membered heterocycle and optionally substituted C₃-C₅ carbocycle, wherein the substituents on each are independently selected at each occurrence from one or more halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, wherein the C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl.

In some aspects, the compound of Formula (X) or Formula (X*) are represented by Formula (I).

In some aspects, the disclosure provides a method of synthesizing a compound represented by Formula (X*):

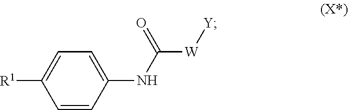

R¹ is selected from:
   —N(R⁵)₂, wherein R⁵ is selected from hydrogen, and optionally substituted C₁-C₆ alkyl, wherein the optional substituents on C₁-C₆ alkyl are independently selected at each occurrence from one or more halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
   optionally substituted C₁-C₆ alkyl, wherein the substituents on C₁-C₆ alkyl are independently selected at each occurrence from one or more halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, 3- to 12-membered heterocycle;
   optionally substituted 3 to 14-membered heterocycle; wherein the optional substituents on the 3 to 8-membered heterocycle are independently selected at each occurrence from one or more halogen, —OH, —CN, —NO₂, —NH₂, oxo, ═S, —S(O₂)NH₂, —C₁₋₁₀ heteroalkyl, —C₁₋₁₀haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the optional substituents on the C₁₋₁₀ alkyl are independently selected at each occurrence from one or more hydroxy, halogen, oxo, —C₁₋₁₀ haloalkyl, —NH₂, —CN, and —NO₂;
W is selected from optionally substituted 5- to 8-membered heterocycle and optionally substituted C₃-C₈ carbocycle, wherein the substituents on each are independently selected at each occurrence from one or more halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, wherein the C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl; and
Y is selected from optionally substituted 5- to 8-membered heterocycle and optionally substituted C₃-C₈ carbocycle, wherein the substituents on each are independently selected at each occurrence from one or more halogen, —OH, —CN, —NO₂, —NH₂, oxo, ═S, —C₁₋₁₀haloalkyl, —O—C₁₋₁₀ alkyl, C₂₋₁₀ alkenyl, C₂₋₁₀ alkynyl, C₃₋₁₂ carbocycle, 3- to 12-membered heterocycle, wherein the C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl.

In some aspects, the disclosure provides a method of synthesizing a compound represented by Formula (X*):

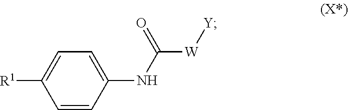

R¹ is selected from:
   —N(R⁵)₂, wherein R⁵ is selected from hydrogen and optionally substituted C₁-C₆ alkyl; optionally substituted C₁-C₆ alkyl;
   optionally substituted 3 to 14-membered heterocycle or optionally substituted 3 to 14-membered carbocycle;
W is selected from optionally substituted 5- to 8-membered heterocycle and optionally substituted C₃-C₈ carbocycle;
and Y is selected from optionally substituted 5- to 8-membered heterocycle and optionally substituted C₃-C₈ carbocycle.

In some embodiments, R is optionally substituted 3 to 14-membered heterocycle.

In some embodiments, provided herein is a method of synthesizing a compound, or a salt thereof, wherein the compound is

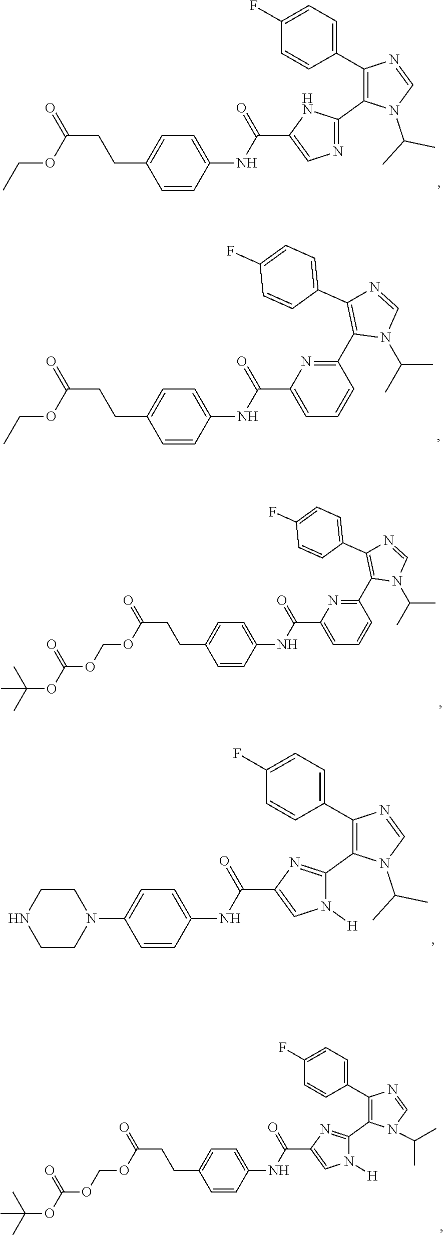

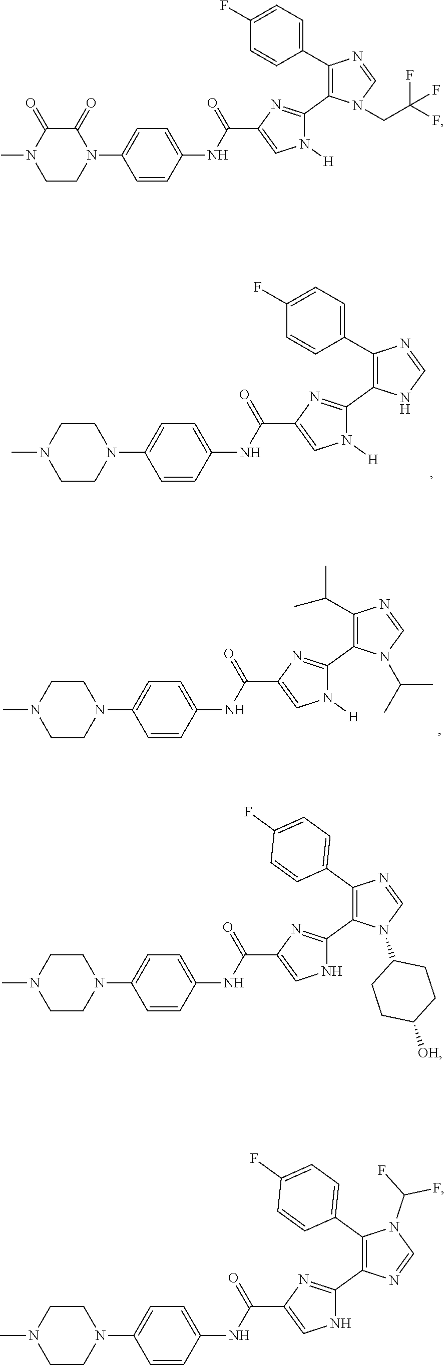

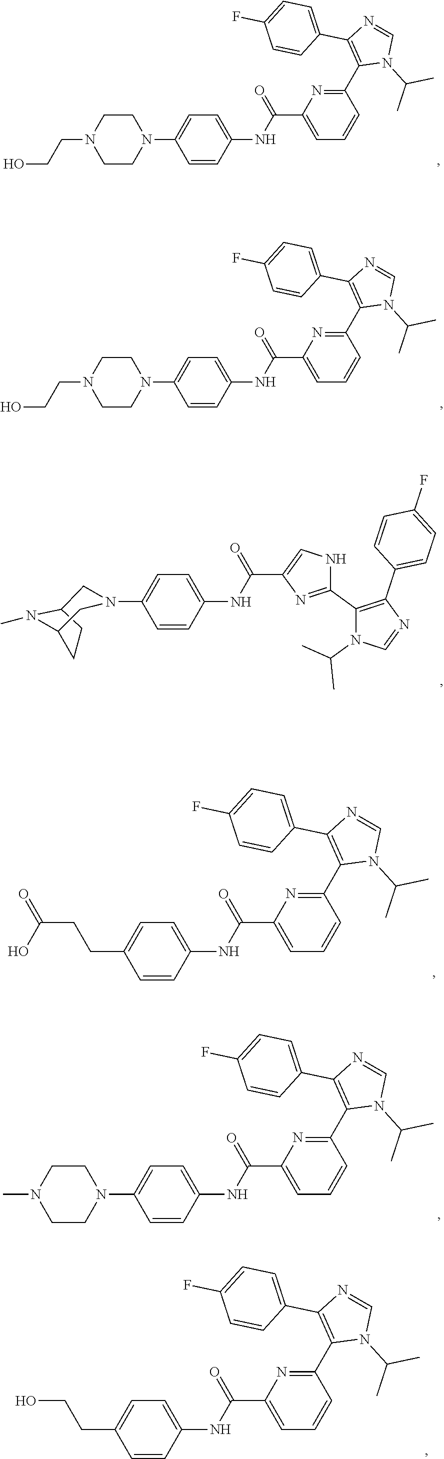

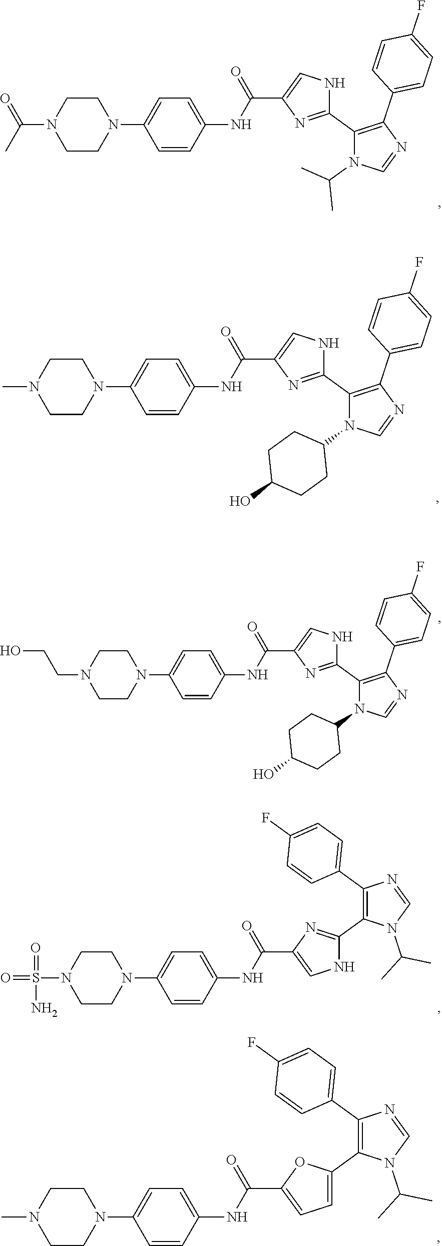

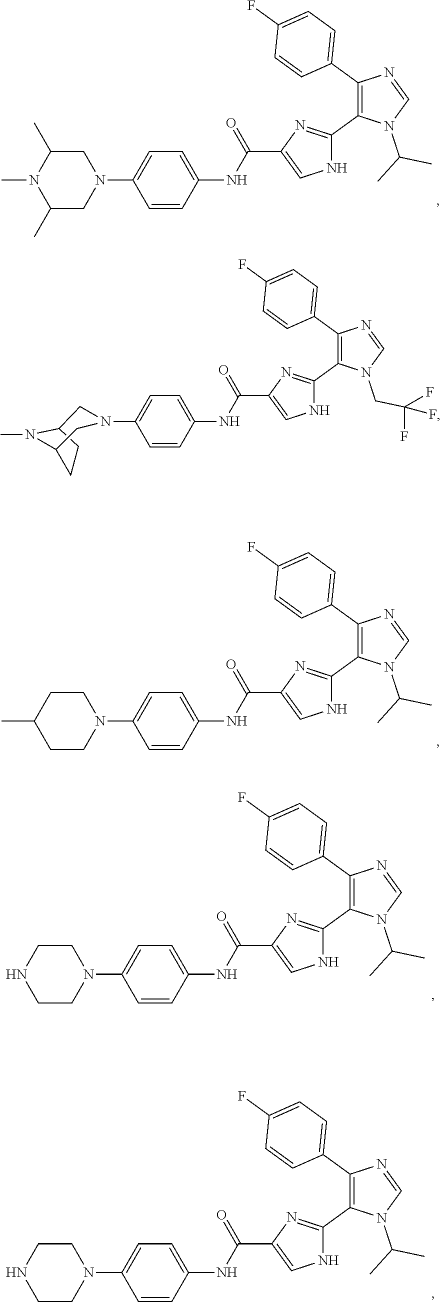

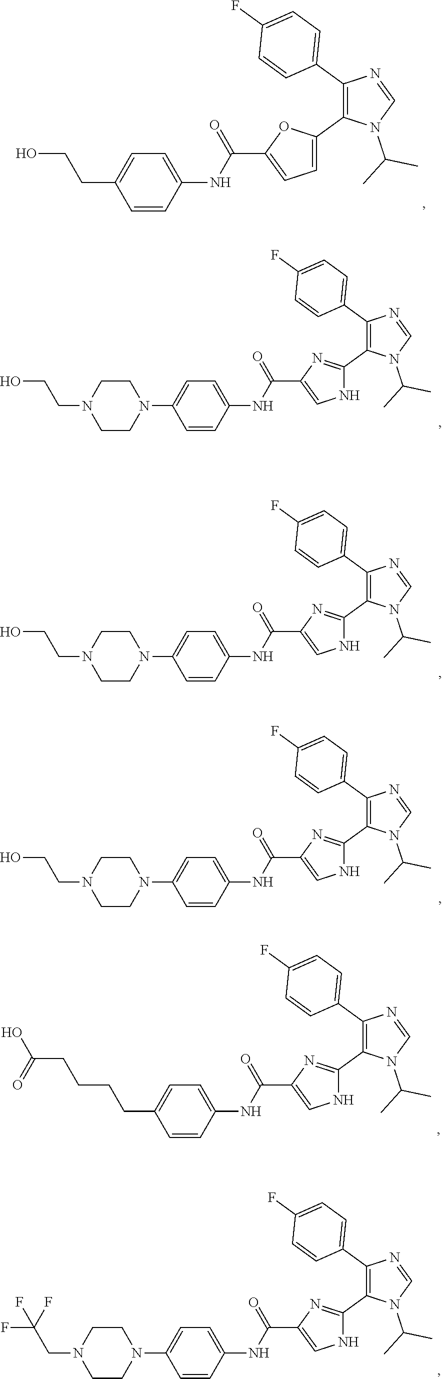 [SMILES: CC(C)n1cnc(-c2ccc(F)cc2)c1-c1ccc(C(=O)Nc2ccc(CCO)cc2)o1 ; CC(C)n1cnc(-c2ccc(F)cc2)c1-c1nc(C(=O)Nc2ccc(N3CCN(CCO)CC3)cc2)c[nH]1 ; CC(C)n1cnc(-c2ccc(F)cc2)c1-c1nc(C(=O)Nc2ccc(N3CCN(CCO)CC3)cc2)c[nH]1 ; CC(C)n1cnc(-c2ccc(F)cc2)c1-c1nc(C(=O)Nc2ccc(N3CCN(CCO)CC3)cc2)c[nH]1 ; CC(C)n1cnc(-c2ccc(F)cc2)c1-c1nc(C(=O)Nc2ccc(CCCCC(=O)O)cc2)c[nH]1 ; CC(C)n1cnc(-c2ccc(F)cc2)c1-c1nc(C(=O)Nc2ccc(N3CCN(CC(F)(F)F)CC3)cc2)c[nH]1]

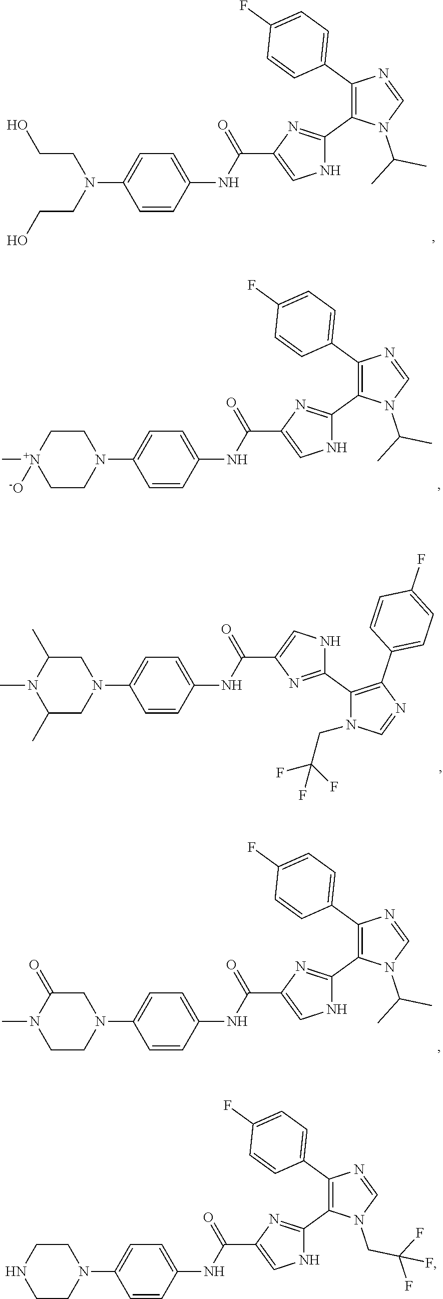

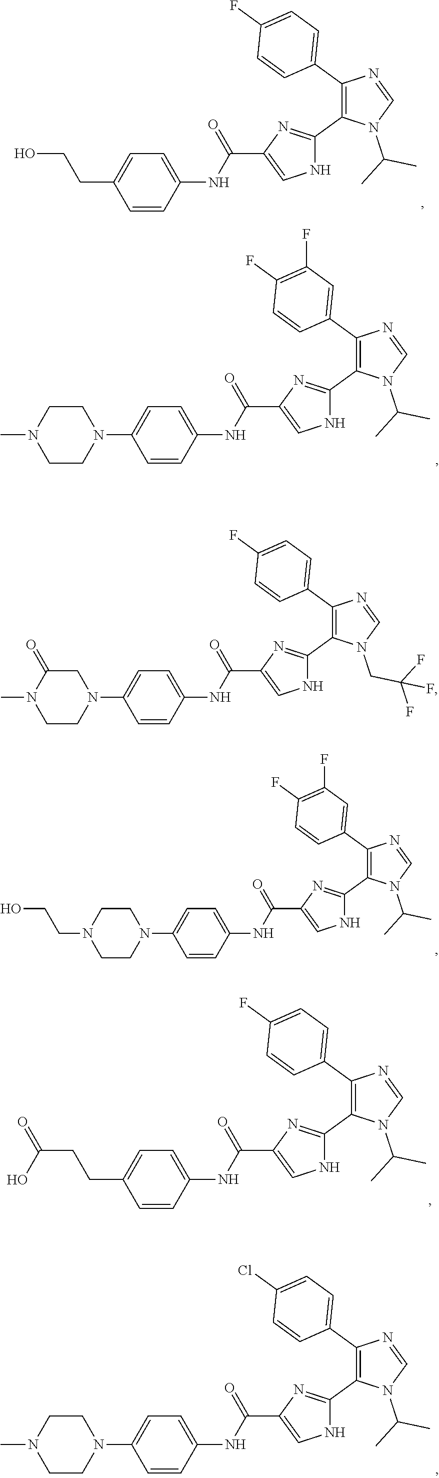

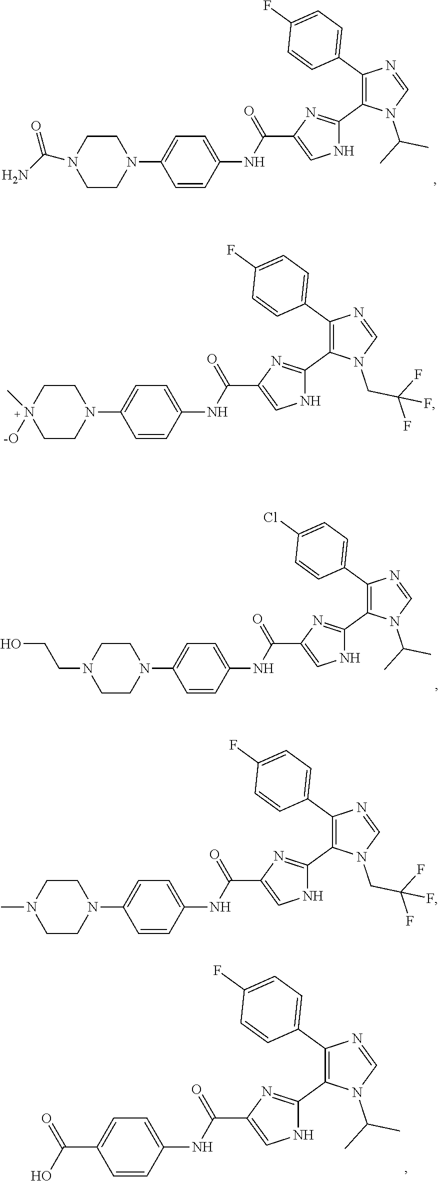

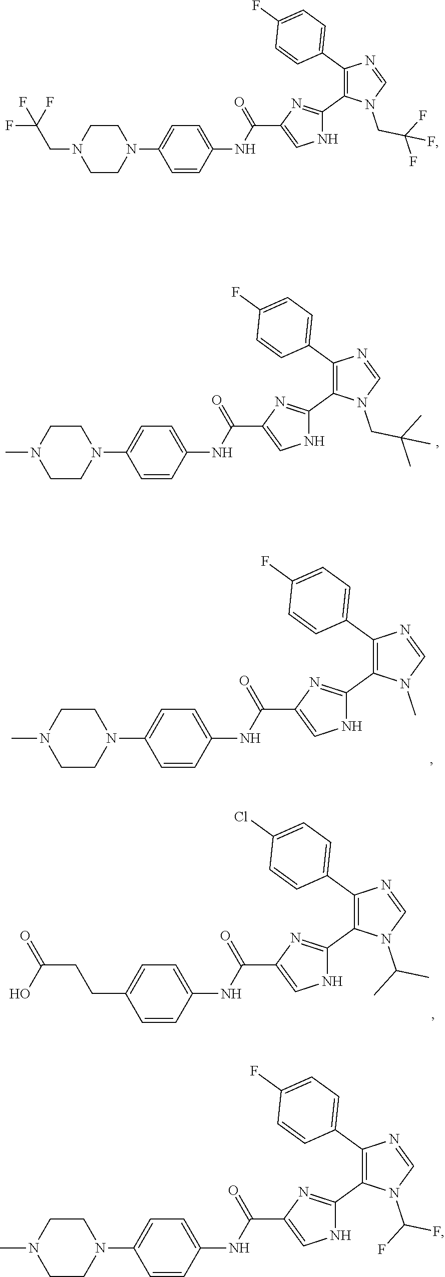

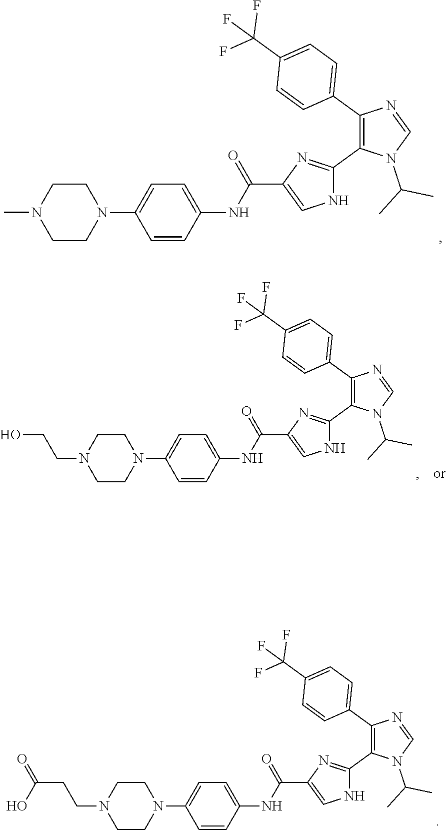 [SMILES: CC(C)n1cnc(-c2ccc(C(F)(F)F)cc2)c1-c1nc(C(=O)Nc2ccc(N3CCN(C)CC3)cc2)c[nH]1 ; CC(C)n1cnc(-c2ccc(C(F)(F)F)cc2)c1-c1nc(C(=O)Nc2ccc(N3CCN(CCO)CC3)cc2)c[nH]1 ; CC(C)n1cnc(-c2ccc(C(F)(F)F)cc2)c1-c1nc(C(=O)Nc2ccc(N3CCN(CCC(=O)O)CC3)cc2)c[nH]1]

In some embodiments, provided herein is a method of synthesizing a compound, or a salt thereof, wherein the compound is

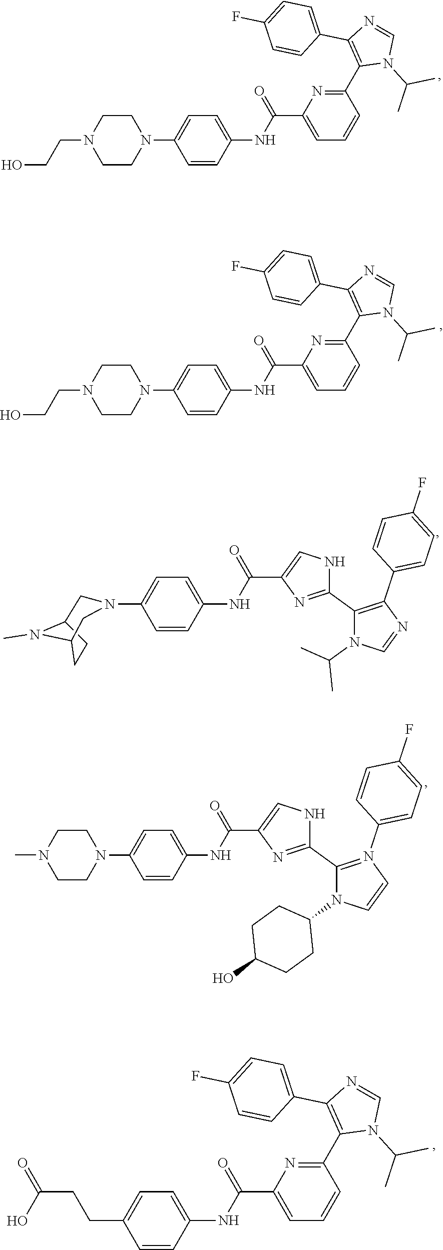

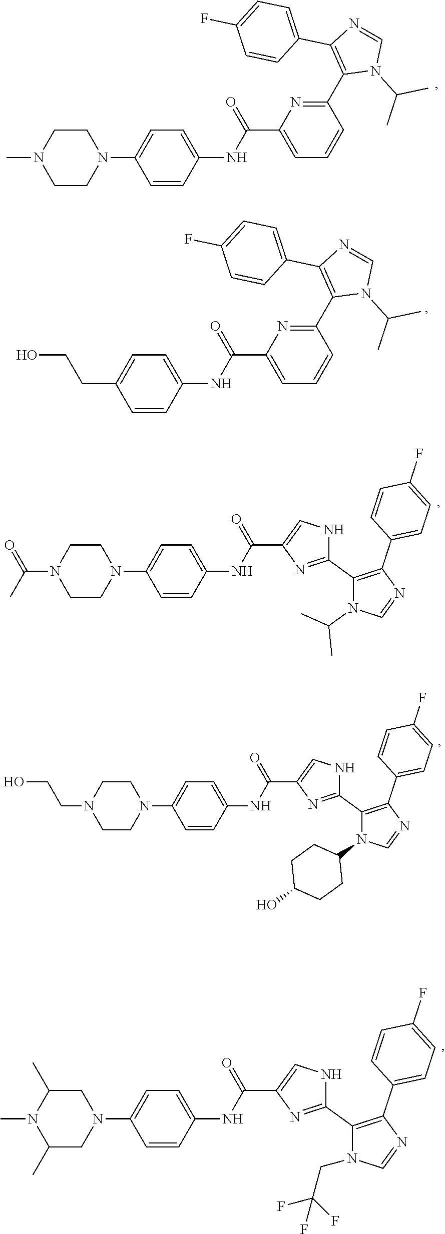

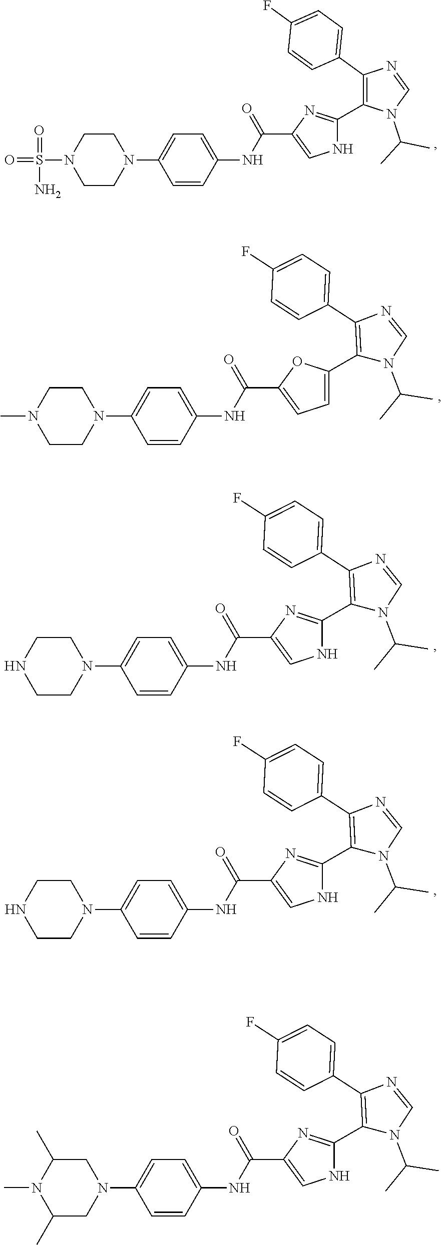

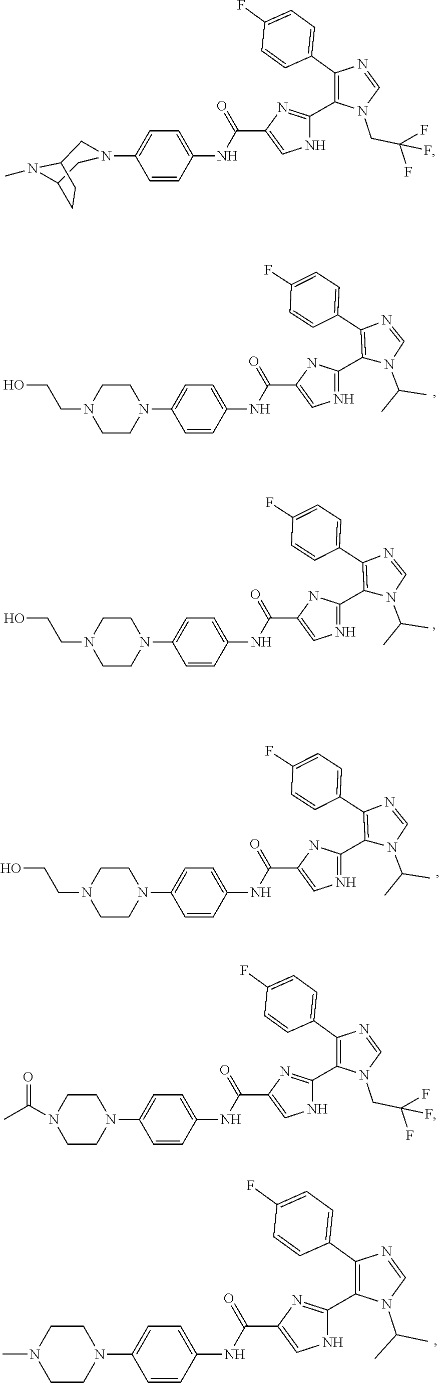

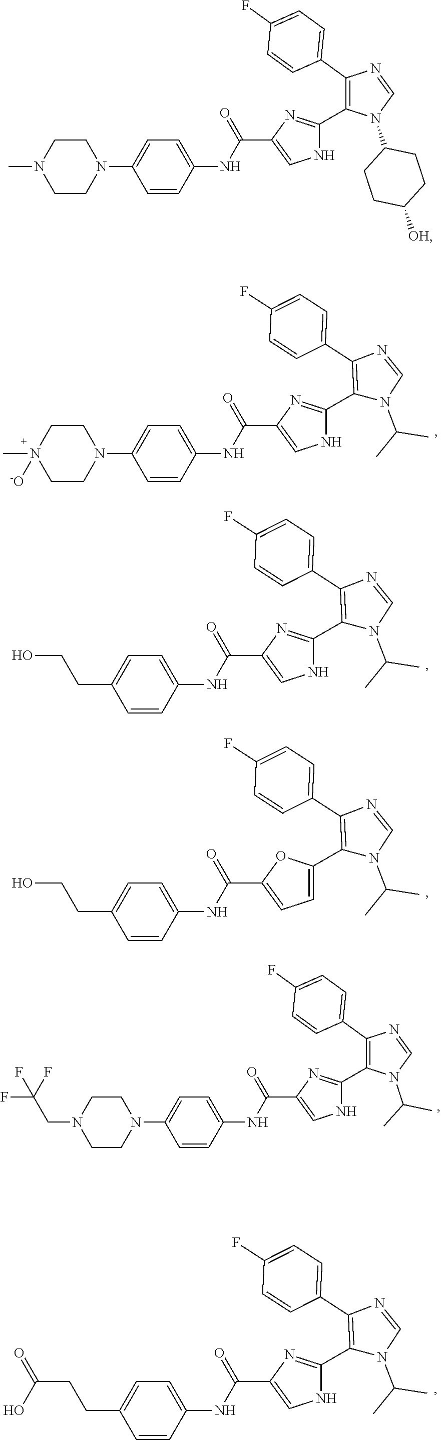

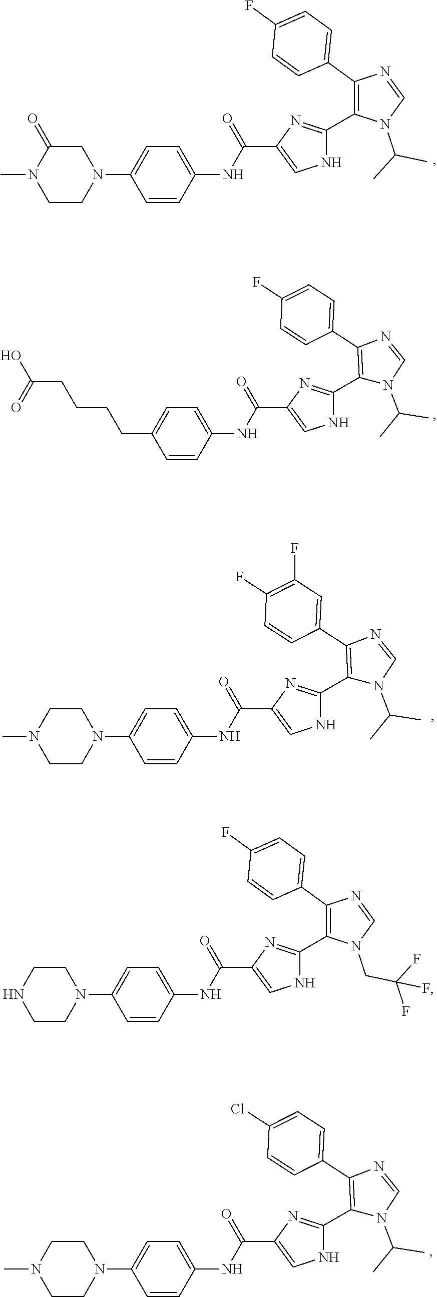

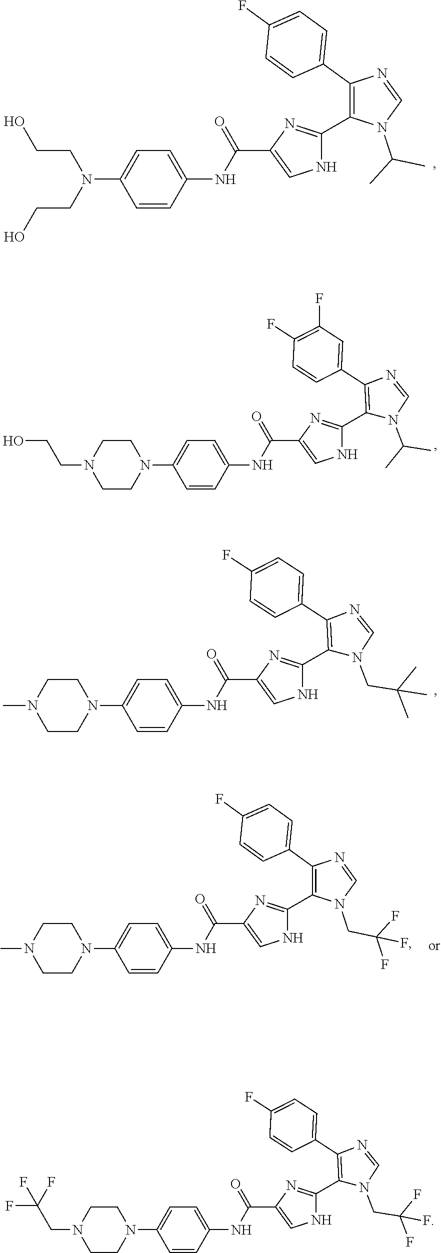

In some embodiments, provided herein is a method of synthesizing a compound, or a salt thereof, wherein the compound is

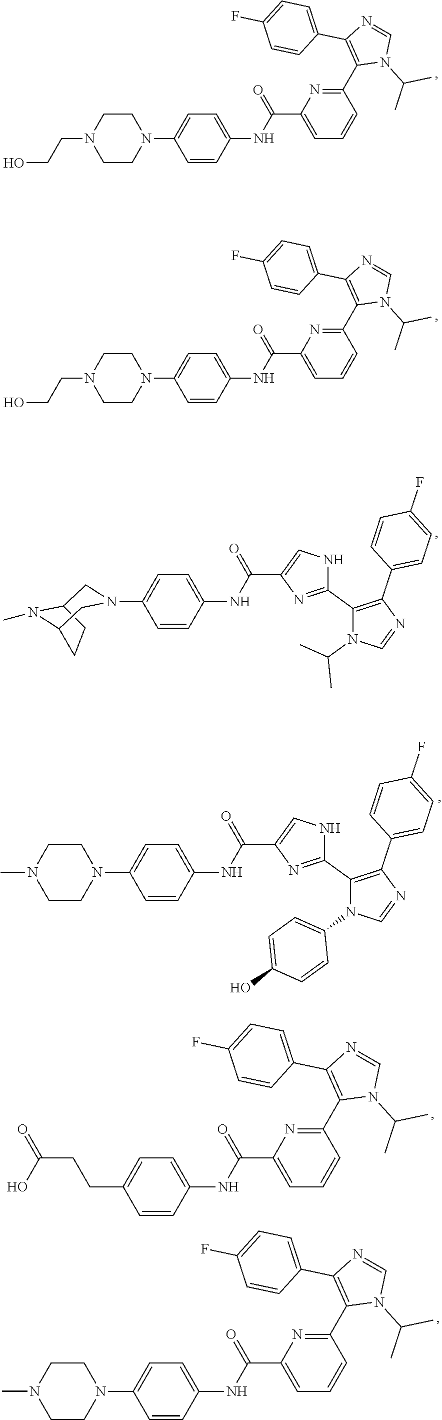

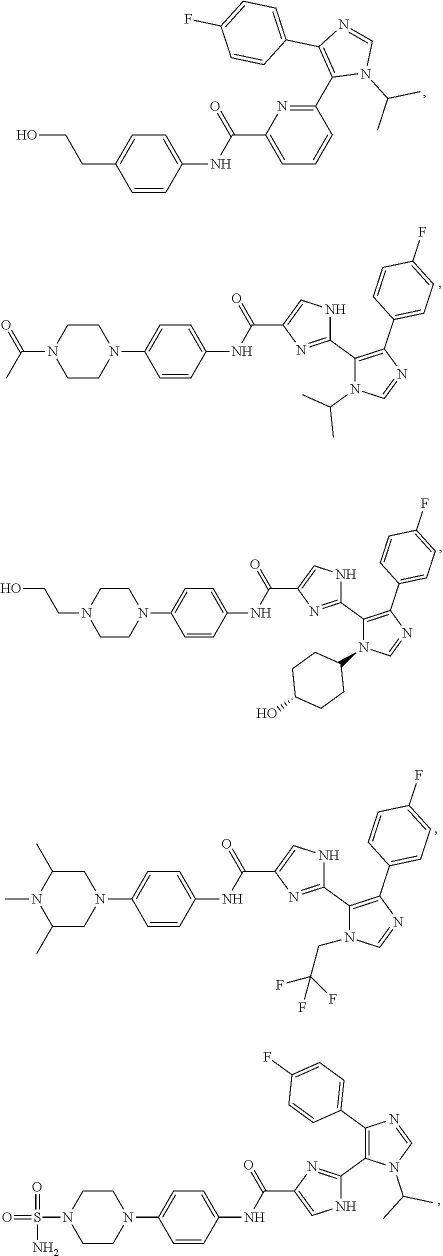

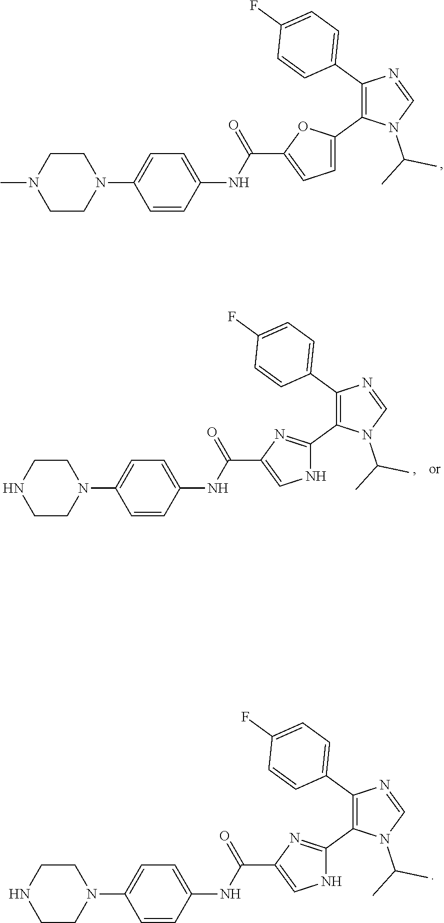 [SMILES: CC(C)n1cnc(-c2ccc(F)cc2)c1-c1ccc(C(=O)Nc2ccc(N3CCN(C)CC3)cc2)o1 ; CC(C)n1cnc(-c2ccc(F)cc2)c1-c1nc(C(=O)Nc2ccc(N3CCNCC3)cc2)c[nH]1 ; CC(C)n1cnc(-c2ccc(F)cc2)c1-c1nc(C(=O)Nc2ccc(N3CCNCC3)cc2)c[nH]1]

In some embodiments, provided herein is a method of synthesizing a compound, or a salt thereof, wherein the compound is

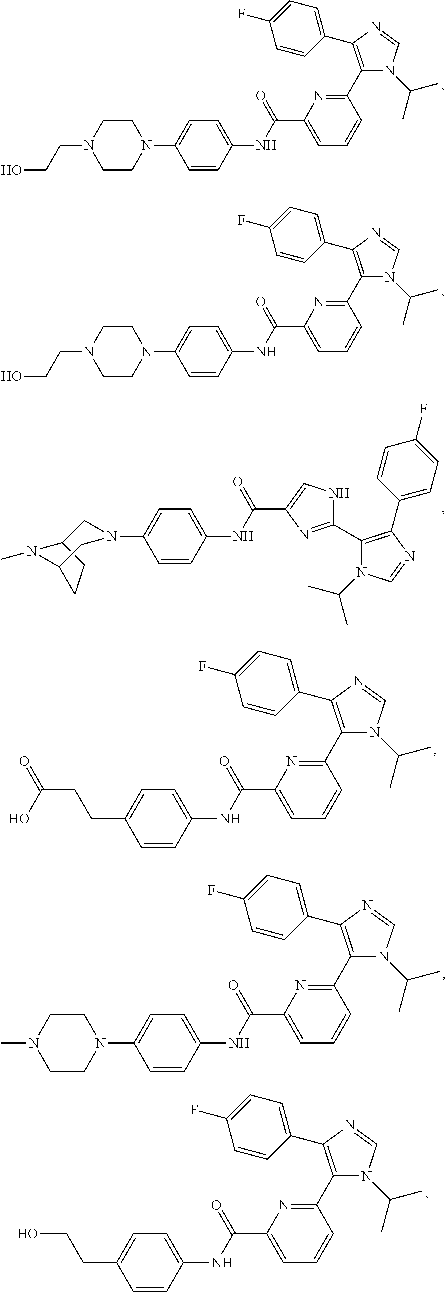

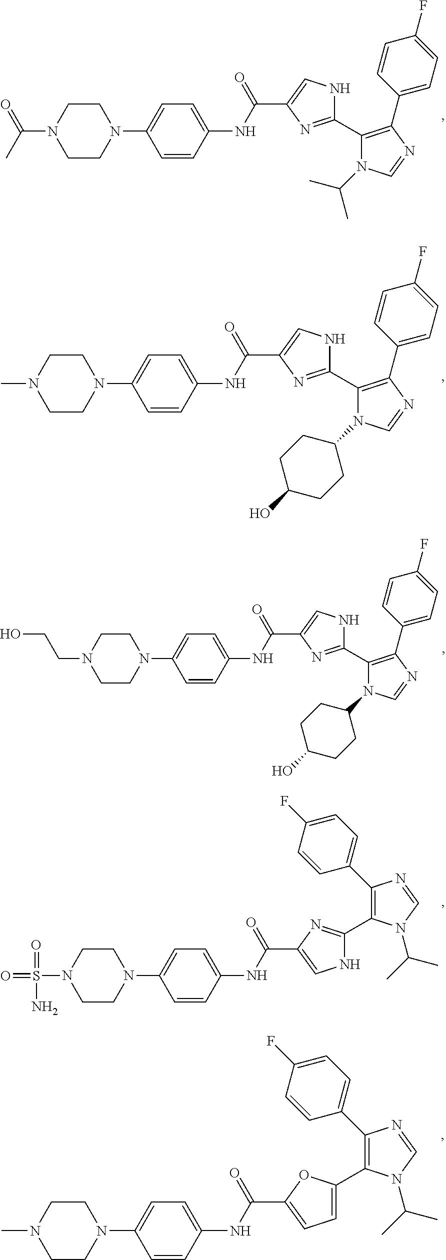

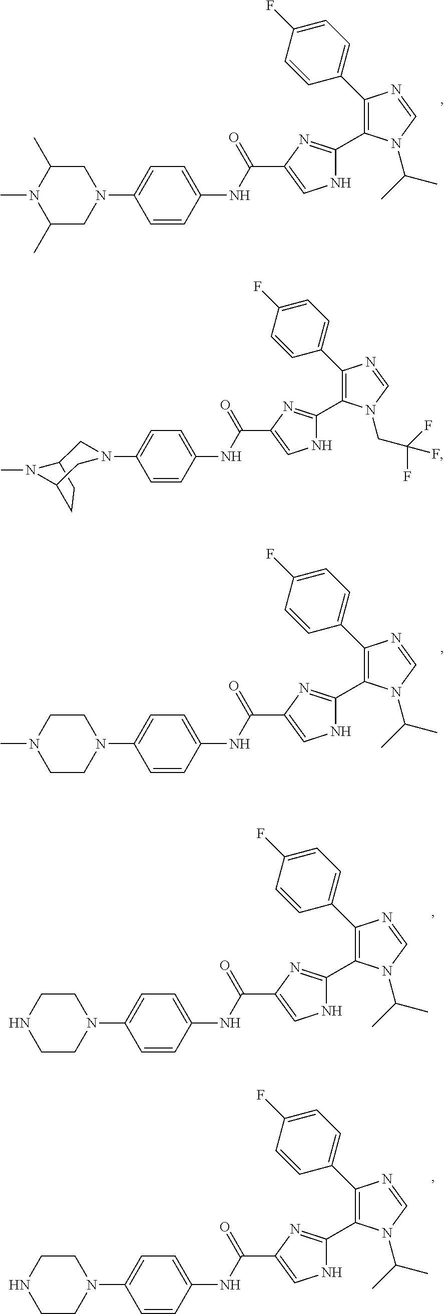

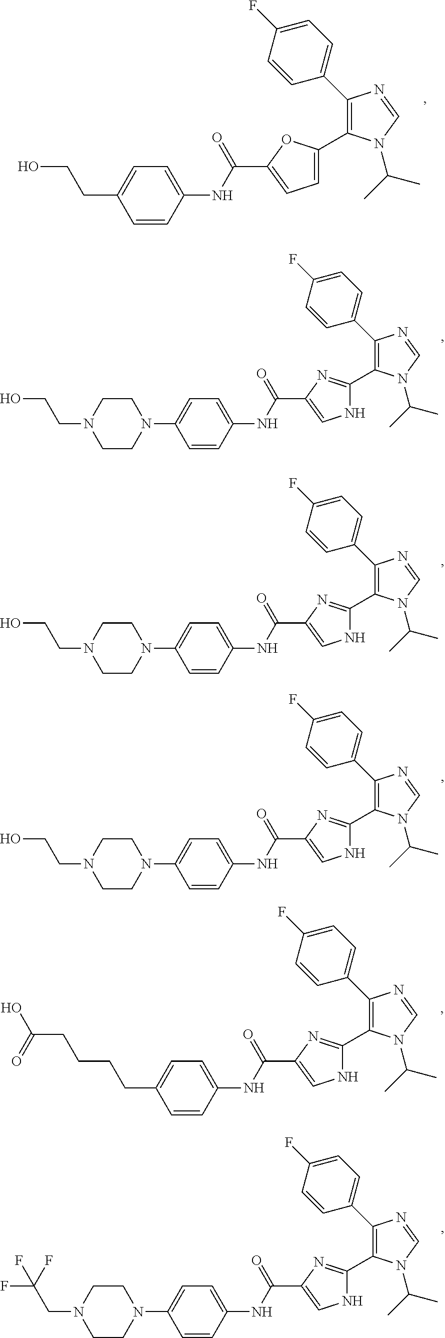 [SMILES: CC(C)n1cnc(-c2ccc(F)cc2)c1-c1ccc(C(=O)Nc2ccc(CCO)cc2)o1 ; CC(C)n1cnc(-c2ccc(F)cc2)c1-c1nc(C(=O)Nc2ccc(N3CCN(CCO)CC3)cc2)c[nH]1 ; CC(C)n1cnc(-c2ccc(F)cc2)c1-c1nc(C(=O)Nc2ccc(N3CCN(CCO)CC3)cc2)c[nH]1 ; CC(C)n1cnc(-c2ccc(F)cc2)c1-c1nc(C(=O)Nc2ccc(N3CCN(CCO)CC3)cc2)c[nH]1 ; CC(C)n1cnc(-c2ccc(F)cc2)c1-c1nc(C(=O)Nc2ccc(CCCCC(=O)O)cc2)c[nH]1 ; CC(C)n1cnc(-c2ccc(F)cc2)c1-c1nc(C(=O)Nc2ccc(N3CCN(CC(F)(F)F)CC3)cc2)c[nH]1]

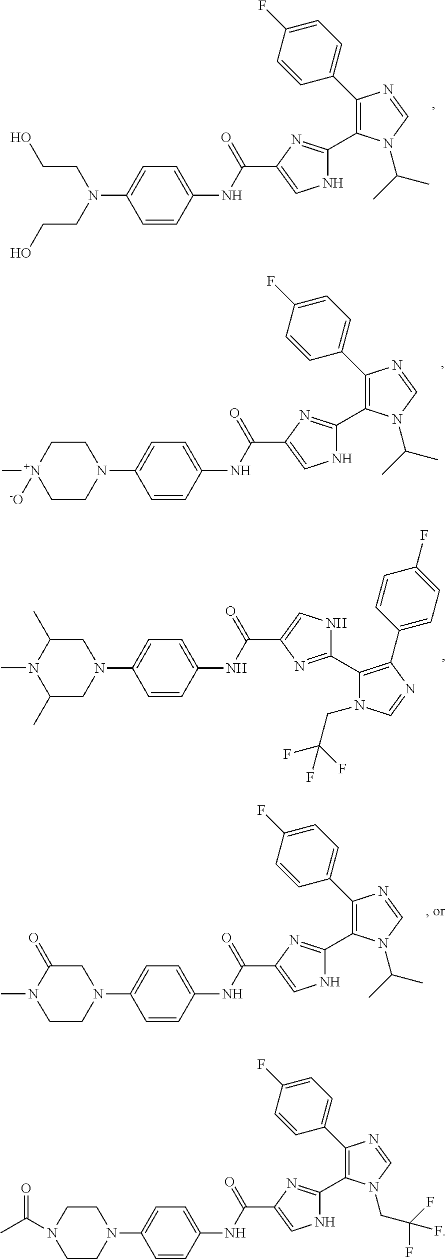

In some embodiments, provided herein is a method of synthesizing a compound, or a salt thereof wherein the compound is

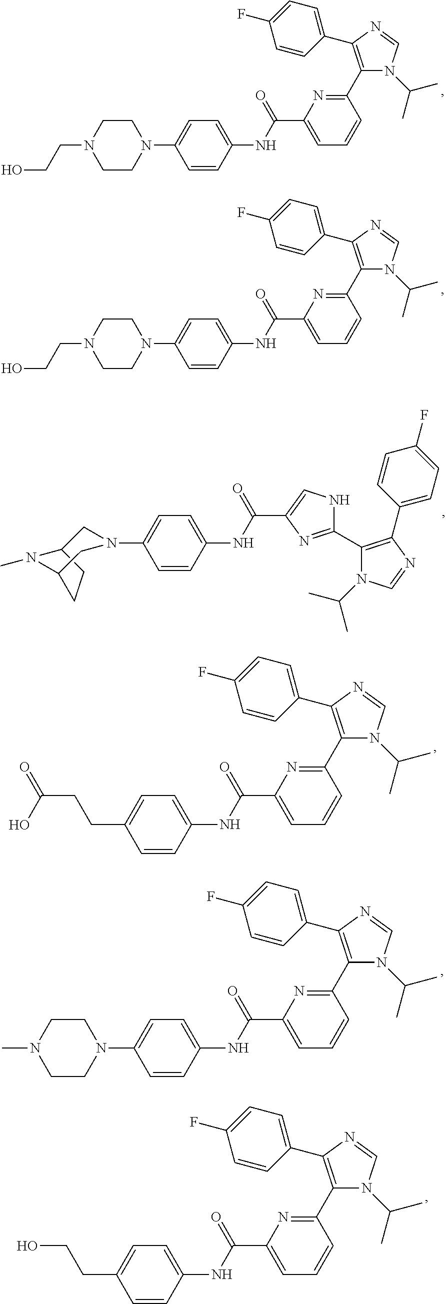

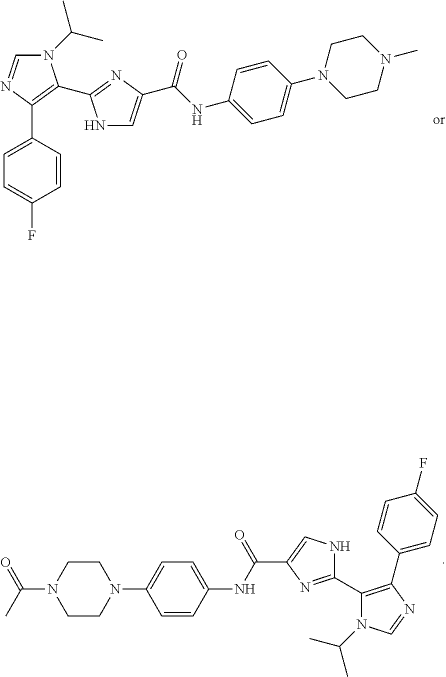

In some embodiments, provided herein is a method of synthesizing a compound, or a salt thereof, wherein the compound is selected from Table 1.

TABLE 1
Exemplary Compounds

| Com- | | Com- | |
|---|---|---|---|
| pound | | pound | |
| # | Compound Structure | # | Compound Structure |
| 172 | 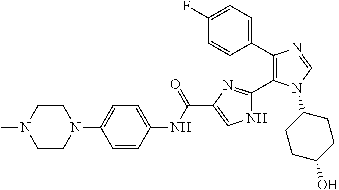 | 111 | 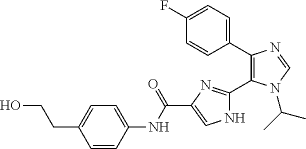 [SMILES: CC(C)n1cnc(-c2ccc(F)cc2)c1-c1nc(C(=O)Nc2ccc(CCO)cc2)c[nH]1] |
| 175 | 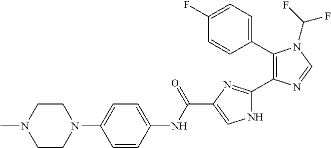 [SMILES: CN1CCN(c2ccc(NC(=O)c3c[nH]c(-c4ncn(C(F)F)c4-c4ccc(F)cc4)n3)cc2)CC1] | 156 | 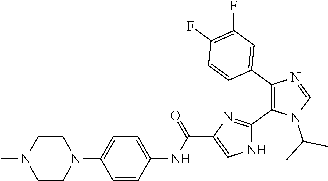 [SMILES: CC(C)n1cnc(-c2ccc(F)c(F)c2)c1-c1nc(C(=O)Nc2ccc(N3CCN(C)CC3)cc2)c[nH]1] |
| 128 | 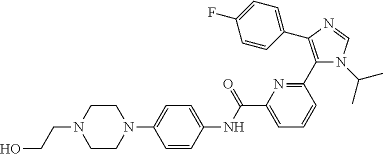 [SMILES: CC(C)n1cnc(-c2ccc(F)cc2)c1-c1cccc(C(=O)Nc2ccc(N3CCN(CCO)CC3)cc2)n1] | 194 | 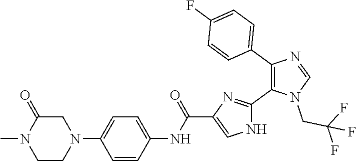 [SMILES: CN1CCN(c2ccc(NC(=O)c3c[nH]c(-c4c(-c5ccc(F)cc5)ncn4CC(F)(F)F)n3)cc2)CC1=O] |
| 186 | 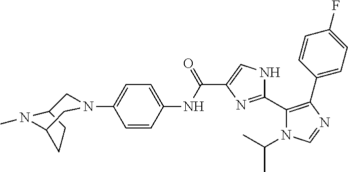 [SMILES: CC(C)n1cnc(-c2ccc(F)cc2)c1-c1nc(C(=O)Nc2ccc(N3CC4CCC(C3)N4C)cc2)c[nH]1] | 146 | 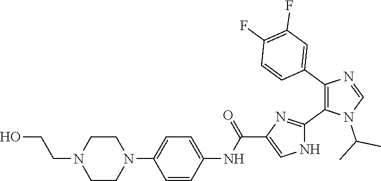 [SMILES: CC(C)n1cnc(-c2ccc(F)c(F)c2)c1-c1nc(C(=O)Nc2ccc(N3CCN(CCO)CC3)cc2)c[nH]1] |
| 131 | 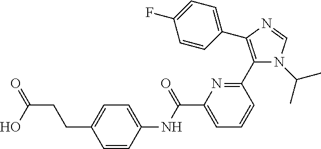 [SMILES: CC(C)n1cnc(-c2ccc(F)cc2)c1-c1cccc(C(=O)Nc2ccc(CCC(=O)O)cc2)n1] | 102 | 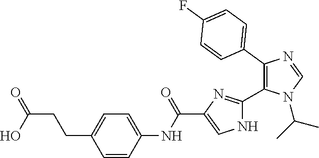 [SMILES: CC(C)n1cnc(-c2ccc(F)cc2)c1-c1nc(C(=O)Nc2ccc(CCC(=O)O)cc2)c[nH]1] |
| 127 | 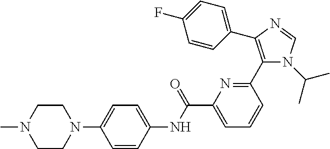 [SMILES: CC(C)n1cnc(-c2ccc(F)cc2)c1-c1cccc(C(=O)Nc2ccc(N3CCN(C)CC3)cc2)n1] | 150 | 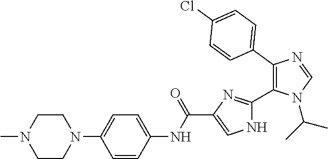 [SMILES: CC(C)n1cnc(-c2ccc(Cl)cc2)c1-c1nc(C(=O)Nc2ccc(N3CCN(C)CC3)cc2)c[nH]1] |
| 125 | 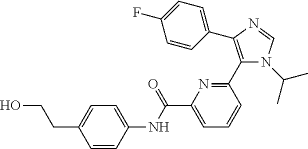 [SMILES: CC(C)n1cnc(-c2ccc(F)cc2)c1-c1cccc(C(=O)Nc2ccc(CCO)cc2)n1] | 99 | 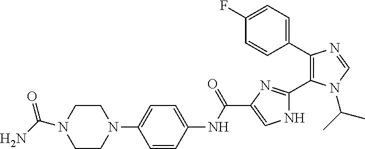 [SMILES: CC(C)n1cnc(-c2ccc(F)cc2)c1-c1nc(C(=O)Nc2ccc(N3CCN(C(N)=O)CC3)cc2)c[nH]1] |
| 188 | 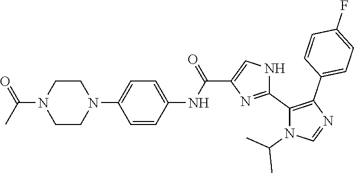 [SMILES: CC(=O)N1CCN(c2ccc(NC(=O)c3c[nH]c(-c4c(-c5ccc(F)cc5)ncn4C(C)C)n3)cc2)CC1] | 166 | 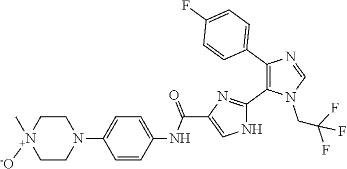 |
| 159 | 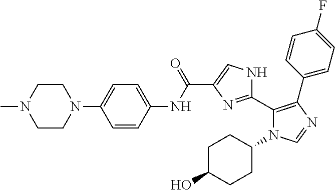 [SMILES: CN1CCN(c2ccc(NC(=O)c3c[nH]c(-c4c(-c5ccc(F)cc5)ncn4[C@H]4CC[C@H](O)CC4)n3)cc2)CC1] | 139 | 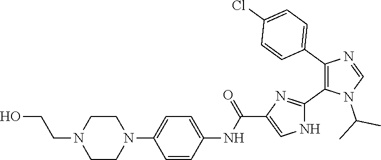 [SMILES: CC(C)n1cnc(-c2ccc(Cl)cc2)c1-c1nc(C(=O)Nc2ccc(N3CCN(CCO)CC3)cc2)c[nH]1] |
| 158 | 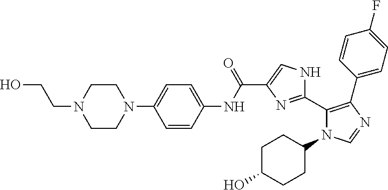 | 120 | 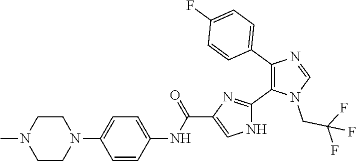 [SMILES: CN1CCN(c2ccc(NC(=O)c3c[nH]c(-c4c(-c5ccc(F)cc5)ncn4CC(F)(F)F)n3)cc2)CC1] |
| 97 | 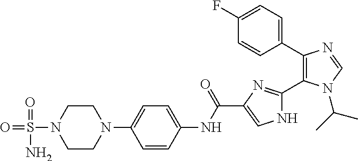 [SMILES: CC(C)n1cnc(-c2ccc(F)cc2)c1-c1nc(C(=O)Nc2ccc(N3CCN(S(N)(=O)=O)CC3)cc2)c[nH]1] | 113 | 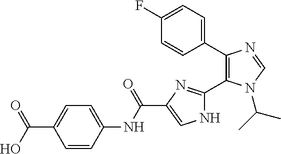 [SMILES: CC(C)n1cnc(-c2ccc(F)cc2)c1-c1nc(C(=O)Nc2ccc(C(=O)O)cc2)c[nH]1] |
| 121 | 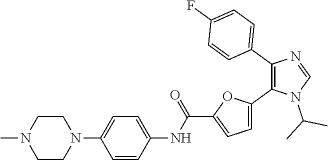 [SMILES: CC(C)n1cnc(-c2ccc(F)cc2)c1-c1ccc(C(=O)Nc2ccc(N3CCN(C)CC3)cc2)o1] | 192 | 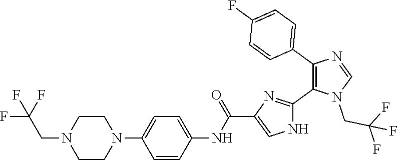 [SMILES: O=C(Nc1ccc(N2CCN(CC(F)(F)F)CC2)cc1)c1c[nH]c(-c2c(-c3ccc(F)cc3)ncn2CC(F)(F)F)n1] |
| 196 | 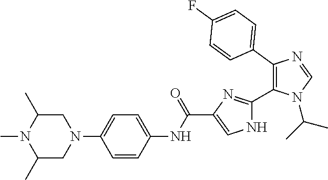 [SMILES: CC1CN(c2ccc(NC(=O)c3c[nH]c(-c4c(-c5ccc(F)cc5)ncn4C(C)C)n3)cc2)CC(C)N1C] | 135 | 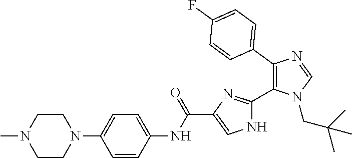 [SMILES: CN1CCN(c2ccc(NC(=O)c3c[nH]c(-c4c(-c5ccc(F)cc5)ncn4CC(C)(C)C)n3)cc2)CC1] |
| 193 | 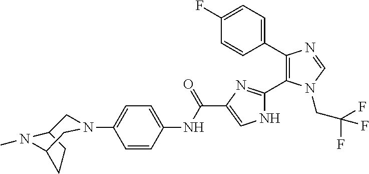 [SMILES: CN1C2CCC1CN(c1ccc(NC(=O)c3c[nH]c(-c4c(-c5ccc(F)cc5)ncn4CC(F)(F)F)n3)cc1)C2] | 119 | 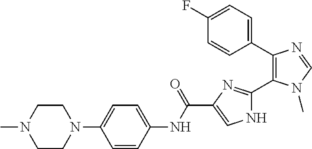 [SMILES: CN1CCN(c2ccc(NC(=O)c3c[nH]c(-c4c(-c5ccc(F)cc5)ncn4C)n3)cc2)CC1] |
| 112 | 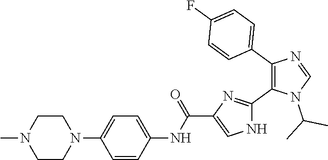 [SMILES: CC(C)n1cnc(-c2ccc(F)cc2)c1-c1nc(C(=O)Nc2ccc(N3CCN(C)CC3)cc2)c[nH]1] | 137 | 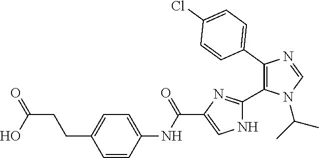 [SMILES: CC(C)n1cnc(-c2ccc(Cl)cc2)c1-c1nc(C(=O)Nc2ccc(CCC(=O)O)cc2)c[nH]1] |
| 181 | 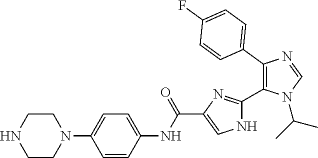 [SMILES: CC(C)n1cnc(-c2ccc(F)cc2)c1-c1nc(C(=O)Nc2ccc(N3CCNCC3)cc2)c[nH]1] | 130 | 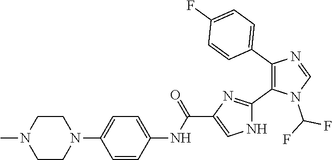 [SMILES: CN1CCN(c2ccc(NC(=O)c3c[nH]c(-c4c(-c5ccc(F)cc5)ncn4C(F)F)n3)cc2)CC1] |
| 126 | 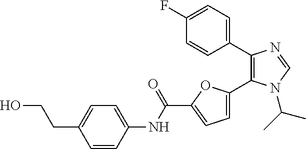 [SMILES: CC(C)n1cnc(-c2ccc(F)cc2)c1-c1ccc(C(=O)Nc2ccc(CCO)cc2)o1] | 142 | 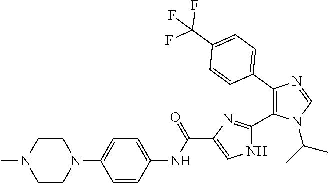 [SMILES: CC(C)n1cnc(-c2ccc(C(F)(F)F)cc2)c1-c1nc(C(=O)Nc2ccc(N3CCN(C)CC3)cc2)c[nH]1] |
| 117 | 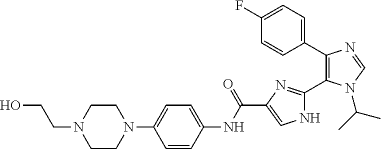 [SMILES: CC(C)n1cnc(-c2ccc(F)cc2)c1-c1nc(C(=O)Nc2ccc(N3CCN(CCO)CC3)cc2)c[nH]1] | 134 | 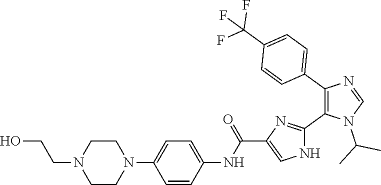 [SMILES: CC(C)n1cnc(-c2ccc(C(F)(F)F)cc2)c1-c1nc(C(=O)Nc2ccc(N3CCN(CCO)CC3)cc2)c[nH]1] |
| 132 | 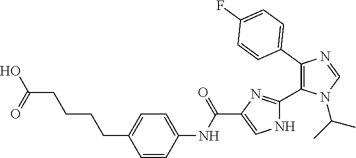 [SMILES: CC(C)n1cnc(-c2ccc(F)cc2)c1-c1nc(C(=O)Nc2ccc(CCCCC(=O)O)cc2)c[nH]1] | 133 | 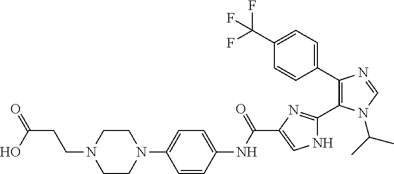 [SMILES: CC(C)n1cnc(-c2ccc(C(F)(F)F)cc2)c1-c1nc(C(=O)Nc2ccc(N3CCN(CCC(=O)O)CC3)cc2)c[nH]1] |
| 189 | 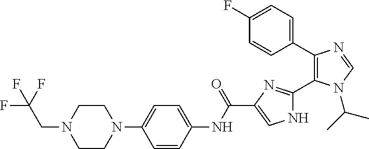 [SMILES: CC(C)n1cnc(-c2ccc(F)cc2)c1-c1nc(C(=O)Nc2ccc(N3CCN(CC(F)(F)F)CC3)cc2)c[nH]1] | 164 | 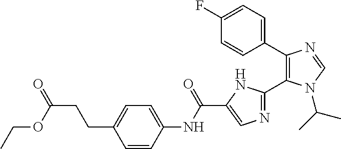 [SMILES: CCOC(=O)CCc1ccc(NC(=O)c2cnc(-c3c(-c4ccc(F)cc4)ncn3C(C)C)[nH]2)cc1] |
| 116 | 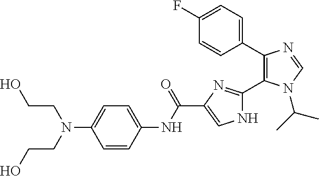 [SMILES: CC(C)n1cnc(-c2ccc(F)cc2)c1-c1nc(C(=O)Nc2ccc(N(CCO)CCO)cc2)c[nH]1] | 168 | 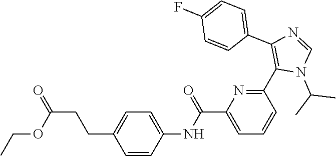 [SMILES: CCOC(=O)CCc1ccc(NC(=O)c2cccc(-c3c(-c4ccc(F)cc4)ncn3C(C)C)n2)cc1] |
| 153 | 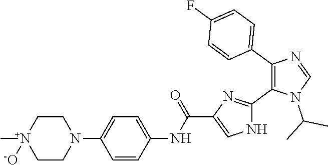 | 171 | 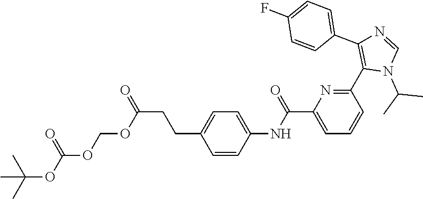 [SMILES: CC(C)n1cnc(-c2ccc(F)cc2)c1-c1cccc(C(=O)Nc2ccc(CCC(=O)OCOC(=O)OC(C)(C)C)cc2)n1] |
| 190 | 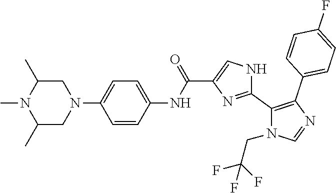 [SMILES: CC1CN(c2ccc(NC(=O)c3c[nH]c(-c4c(-c5ccc(F)cc5)ncn4CC(F)(F)F)n3)cc2)CC(C)N1C] | 198 | 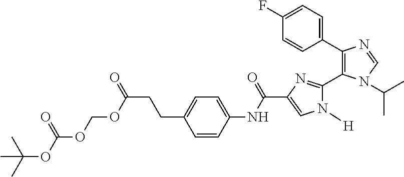 [SMILES: CC(C)n1cnc(-c2ccc(F)cc2)c1-c1nc(C(=O)Nc2ccc(CCC(=O)OCOC(=O)OC(C)(C)C)cc2)c[nH]1] |
| 185 | 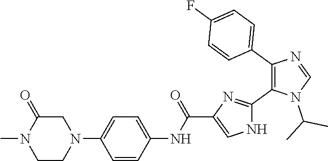 [SMILES: CC(C)n1cnc(-c2ccc(F)cc2)c1-c1nc(C(=O)Nc2ccc(N3CCN(C)C(=O)C3)cc2)c[nH]1] | 199 | 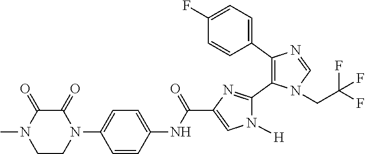 [SMILES: CN1CCN(c2ccc(NC(=O)c3c[nH]c(-c4c(-c5ccc(F)cc5)ncn4CC(F)(F)F)n3)cc2)C(=O)C1=O] |
| 191 | 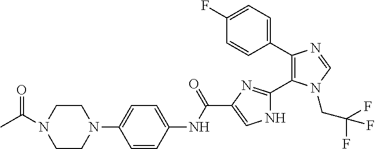 [SMILES: CC(=O)N1CCN(c2ccc(NC(=O)c3c[nH]c(-c4c(-c5ccc(F)cc5)ncn4CC(F)(F)F)n3)cc2)CC1] | 200 | 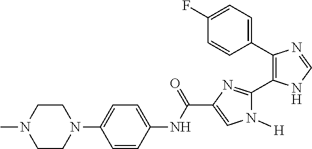 [SMILES: CN1CCN(c2ccc(NC(=O)c3c[nH]c(-c4[nH]cnc4-c4ccc(F)cc4)n3)cc2)CC1] |
| 167 | 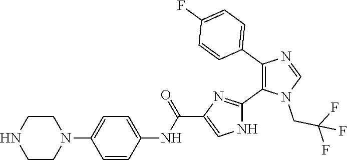 [SMILES: O=C(Nc1ccc(N2CCNCC2)cc1)c1c[nH]c(-c2c(-c3ccc(F)cc3)ncn2CC(F)(F)F)n1] | 205 | 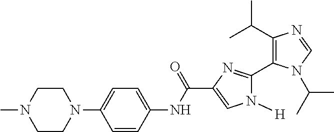 [SMILES: CC(C)c1ncn(C(C)C)c1-c1nc(C(=O)Nc2ccc(N3CCN(C)CC3)cc2)c[nH]1] |

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

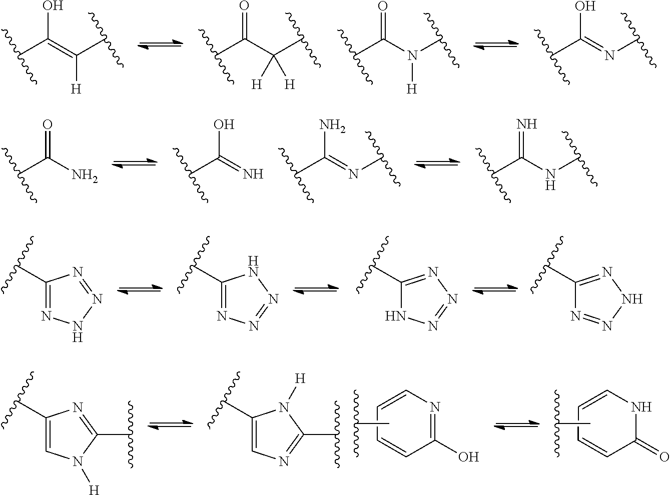

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³S, ³⁶S, ³⁵Cl, ³⁷Cl, ⁷⁹Br, ⁸¹Br, and ¹²⁵I are all contemplated. All isotopic variations of the compounds of the present disclosure, whether radioactive or not, are encompassed within the scope of the present disclosure. In some embodiments, where isotopic variations are illustrated, the remaining atoms of the compound may optionally contain unnatural portions of atomic isotopes.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

In some embodiments of a compound disclosed herein, one or more of R¹, R³, R⁴, R⁵, R^{A}, R^{A2}, R^{A3}, R^{EN}, R^{EN′}, R^{F}, R^{F1}, R^{E1}, R^{J}, W, Z, Y, and R¹⁰ groups comprise deuterium at a percentage higher than the natural abundance of deuterium.

In some embodiments of a compound disclosed herein, one or more ¹H are replaced with one or more deuteriums in one or more of the following groups R¹, R³, R⁴, R⁵, R^{A1}, R^{A2}, R^{A3}, R^{EN}, R^{EN′}, R^{F}, R^{F1}, R^{E1}, R, W, Z, Y, and R¹⁰.

In some embodiments of a compound disclosed herein, the abundance of deuterium in each of R¹, R³, R⁴, R⁵, R^{A1}, R^{A2}, R^{A3}, R^{EN}, R^{EN′}, R^{F}, R^{F1}, R^{E1}, R^{J}, W, Z, Y, and R¹⁰ is independently at least 1%, at least 10%, at least 20%, at least 30%, at least 40%, at least 50%, at least 60%, at least 70%, at least 80%, at least 90%, or 100% by molar.

In some embodiments of a compound disclosed herein, one or more ¹H of ring W are replaced with one or more deuteriums.

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may in some cases exist as diastereomers, enantiomers, or other stereoisomeric forms. Where absolute stereochemistry is not specified, the compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrug forms of the herein described compounds, wherein the prodrug is metabolized in vivo to produce a compound as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not.

Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### D. METHODS OF TREATMENT

The compounds synthesized by methods described herein can be used in the preparation of medicaments for the prevention or treatment of diseases or conditions. In addition, a method for treating any of the diseases or conditions described herein in a subject in need of such treatment, involves administration of pharmaceutical compositions containing at least one compound described herein, or a pharmaceutically acceptable salt, pharmaceutically acceptable prodrug, or pharmaceutically acceptable solvate thereof, in therapeutically effective amounts to said subject.

In an aspect provided herein, the disclosure provides for methods of synthesizing inhibitors of TNIK kinase. Accordingly, the TNIK kinase inhibitors can be used to inhibit a biological pathway downstream from inhibiting TNIK. In some aspects, the TNIK inhibitor can inhibit fibrillar collagen, and thereby can inhibit biological activity related to regulation of the extracellular matrix, and regulation of remodeling the extracellular matrix. The TNIK inhibitor can inhibit regulation of cell growth, differentiation, cell migration, proliferation, and metabolism.

In certain embodiments, the disclosure provides a method of synthesizing a compound that can be used in treating or preventing a disease, state or condition in a patient in need thereof comprising administering to the patient an effective amount of a compound of any one of embodiments of the disclosure or a pharmaceutically acceptable salt thereof. The disease, state or condition may be selected from the group consisting of colorectal cancer, gastric cancer, breast cancer, lung cancer, pancreatic cancer, prostate cancer, multiple myeloma, chronic myelogenous leukemia, cancer metastasis, fibrosis and psychiatric disorders. In some embodiments, the cancer is colorectal cancer. In some embodiments, the cancer is gastric cancer. In some embodiments, the cancer is breast cancer. In some embodiments, the cancer is lung cancer. In some embodiments, the cancer is pancreatic cancer. In some embodiments, the cancer is prostate cancer. In some embodiments, the cancer is multiple myeloma. In some embodiments, the cancer is chronic myelogenous leukemia. In some embodiments, the cancer is cancer metastasis. In certain embodiments, the cancer is a solid tumor. In certain embodiments, the cancer is not a solid tumor.

In certain embodiments, the inhibiting of TNIK inhibits embryonic development. As such, the TNIK inhibitor can inhibit pregnancy progression and thereby be used for terminating a pregnancy. In some embodiments, the inhibiting of TNIK inhibits TGF beta signaling. The TGF beta signaling pathway is involved in a various processes, and thereby inhibiting the TGF beta signaling pathway can inhibit these processes, some of which are described herein. This can include inhibiting development of an embryo as described herein for inhibiting progression of pregnancy. This can include inhibiting cell growth, cell differentiation, which may be used to inhibit pregnancy progression as well as inhibiting cancer.

In certain embodiments, the disclosure provides for methods of synthesizing a compound that can be used for treating or preventing a fibrotic disease or condition. In some embodiments, the fibrotic disease or condition is selected from pulmonary fibrosis, cystic fibrosis, liver fibrosis, myocardial fibrosis, kidney fibrosis, brain fibrosis, arterial fibrosis, arthrofibrosis, intestinal fibrosis, Dupytren's contracture fibrosis, keloid fibrosis, mediastinal fibrosis, myelofibrosis, peyronie's disease fibrosis, progressive massive fibrosis, retroperitoneal fibrosis, scleroderma sclerosis fibrosis, adhesive capsulitis fibrosis, or combinations thereof. In some embodiments, the fibrotic disease is selected from liver cirrhosis, pulmonary fibrosis, renal interstitial fibrosis, myocardial infarction, systemic sclerosis (SSc), and graft-versus-host disease (GVHD). In some embodiments, the fibrotic disease is kidney fibrosis.

In certain embodiments, the disclosure provides for methods of synthesizing a compound that can be used for treating a kidney disease. In some embodiments, the kidney disease is chronic kidney fibrosis (CKD). In some embodiments, the kidney disease is a kidney fibrosis. In some embodiments, the fibrotic disease is liver cirrhosis. In some embodiments, the fibrotic disease is pulmonary fibrosis. In some embodiments, the fibrotic disease is idiopathic pulmonary fibrosis (IPF). In some embodiments, the fibrotic disease is kidney fibrosis wherein the disease is chronic or acute. In some embodiments, the kidney fibrosis causes glomerulosclerosis or tubulointerstitial fibrosis. In some embodiments, the fibrotic disease is renal interstitial fibrosis. In some embodiments, the fibrotic disease is acute interstitial nephritis (AIN). In some embodiments, the fibrotic disease is systemic sclerosis (SSc). In some embodiments, the fibrotic disease is graft-versus-host disease (GVHD). In some embodiments, the fibrotic disease is hypertrophic scarring (HTS).

### EXAMPLES

The following examples are offered to illustrate, but not to limit the claimed invention. The following examples further illustrate the invention but, of course, should not be construed as in any way limiting its scope.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

In the examples and throughout the applications, certain abbreviations are used. They include:
DIEA=N,N-Diisopropylethylamine;
PE=Petroleum ether;
EA=Ethyl acetate;
¹H-NMR=Proton Nuclear magnetic resonance spectroscopy;
DMA=N,N-Dimethylacetamide;
DCM=Dichloromethane;
NIS=N-Iodosuccinimide;
TFA=Trifluoroacetic acid;
DMSO=Dimethyl sulfoxide;
THF=Tetrahydrofuran;
NBS=N-Bromosuccinimide;
AIBN=azodiisobutyronitrile;
TLC=thin-layer chromatography;
DMF=N,N-Dimethylformamide;
SEM=(trimethyl silicon) ethoxymethyl
HATU=2-(7-Azabenzotriazol-1-yl)-N,N,N′,N′-tetramethyluronium hexafluorophosphate;
2-MeTHF=2-Methyltetrahydrofuran;
MTBE=Methyl tert-butyl ether;
T3P=2,4,6-Tripropyl-1,3,5,2,4,6-Trioxatriphosphorinane-2,4,6-Trioxide;
MCH=Methylcyclohexane;
HPLC=High Performance Liquid Chromatography.

### Illustrative Synthesis Schemes

The compounds and salts of Formulas (I), (Ia), or (IIB) can be synthesized according to one or more illustrative schemes herein and/or techniques known in the art. Materials used herein are either commercially available or prepared by synthetic methods generally known in the art. These schemes are not limited to the compounds listed in the examples or by any particular substituents, which are employed for illustrative purposes. Although various steps are described and depicted in the synthesis schemes below, the steps in some cases may be performed in a different order than the order shown below. Numberings or R groups in each scheme do not necessarily correspond to that of the claims or other schemes or tables herein.

### Example 1. Reference Procedure for Compound 112 (Stille Coupling)

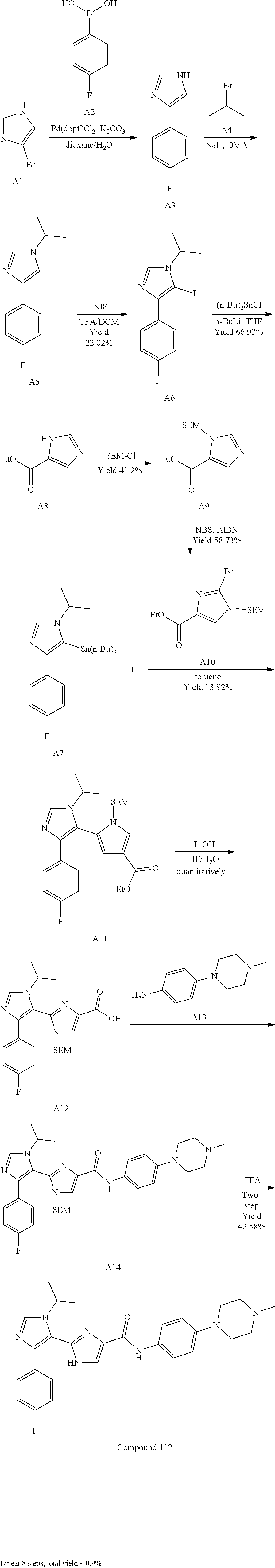

In some cases, the reference procedure of example 1 presents challenges in quality control due to number of steps and/or the use Sn reagent.

### Step 1-1: General Procedure for Preparation of (4-(4-fluorophenyl)-1H-imidazole (Compound A3)

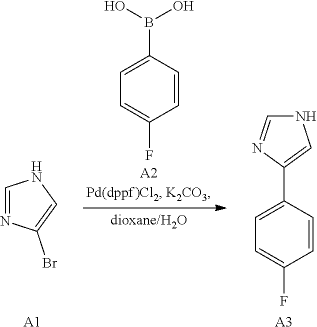

To a solution of 4-bromo-1H-imidazole (compound A1) (60 g, 408.24 mmol, 1 eq) and (4-fluorophenyl)boronic acid (Compound A2) (114.24 g, 816.48 mmol, 2 eq) in dioxane (500 mL) and H₂O (100 mL) was added K₂CO₃ (169.26 g, 1.22 mol, 3 eq) and Pd(dppf)Cl₂ (35.85 g, 48.99 mmol, 0.12 eq) at 30 °C. The mixture was stirred at 110 °C for 16 h. LCMS showed compound A1 was consumed and 57% of desired mass was detected. The mixture was combined with other 3 batches. The mixture was poured to H₂O (1000 mL). The mixture was extracted with EA (1000 mL×2). The combined organic phase was poured to 1N HCl (1000 mL). The mixture was extracted with ethyl acetate (1000 mL×2). The aqueous phase was basified with Na₂CO₃ to pH=8. The mixture was extracted with ethyl acetate (1000 mL×3). The combined organic phase was concentrated in vacuo to afford crude product. Compound A3 (102 g, crude) was obtained as brown solid, which was determined by ¹H-NMR. ¹H-NMR: (400 MHz, DMSO-d₆), δ=7.85-7.76 (m, 2H), 7.73 (d, J=0.9 Hz, 1H), 7.57 (d, J=0.7 Hz, 1H), 7.27-7.12 (m, 2H).

### Step 1-2: General Procedure for Preparation of 4-(4-fluorophenyl)-1-isopropyl-1H-imidazole (Compound A5)

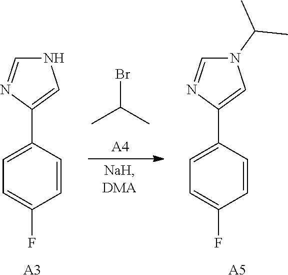

To a solution of NaH (49.08 g, 1.23 mol, 60% purity, 2 eq) in DMA (1000 mL) was added compound A3 (99.5 g, 613.58 mmol, 1 eq) at 0 °C. The mixture was stirred at 0 °C for 0.5 h. 2-Bromopropane (Compound A4) (113.20 g, 920.37 mmol, 86.41 mL, 1.5 eq) was added to the mixture at 0 °C. The mixture was stirred at 80 °C for 16 h. LCMS showed 20% of compound A3 remained and 71% of desired mass was detected. The mixture was poured to aq NH₄Cl (2 L). The mixture was extracted with ethyl acetate (2000 mL×2). The combined organic phase was concentrated in vacuo to afford crude product. Compound A5 (140 g, crude) was obtained as brown oil, which was determined by LCMS and ¹H-NMR.

LCMS: Retention time: 0.521 min, (M+H)=205.1. ¹H-NMR: (400 MHz, CHLOROFORM-d) δ=7.76-7.71 (m, 2H), 7.57 (d, J=1.2 Hz, 1H), 7.21 (d, J=1.2 Hz, 1H), 7.11-7.05 (m, 2H), 4.36 (td, J=6.7, 13.4 Hz, 1H), 1.53 (d, J=6.7 Hz, 6H).

### Step 1-3: General Procedure for Preparation of 4-(4-fluorophenyl)-5-iodo-1-isopropyl-1H-imidazole (Compound A6)

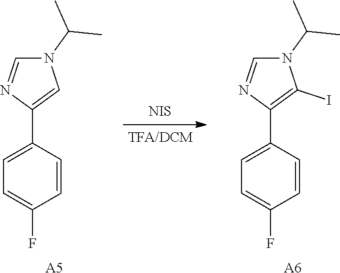

To a solution of compound A5 (136 g, 665.87 mmol, 1 eq) of DCM (1 L) was added NIS (449.42 g, 2.00 mol, 3.0 eq) and TFA (22.78 g, 199.76 mmol, 14.79 mL, 0.3 eq), it was stirred at 25 TC for 16 h. TLC (PE:EA 3:1) showed compound A5 (Rf=0.1) was consumed and a new spot (Rf=0.4) was detected. LCMS showed compound A5 was consumed and desired mass was detected. Saturated Na₂SO₃ aqueous (1 L) was added, the organic layer was washed with brine (1 L), dried over Na₂SO₄, filtered and concentrated in vacuum. The crude was purified by column chromatography (SiO₂, PE:EA 20:1~2:1). Compound A6 (75 g, 227.18 mmol, 34.12% yield) was obtained as a white solid, which was confirmed by LCMS and ¹H-NMR. LCMS: Retention time: 0.729 min, (M+H)=331.0. ¹H-NMR: (400 MHz, DMSO-d₆) δ=8.15 (s, 1H), 7.95-7.84 (m, 2H), 7.32-7.20 (m, 2H), 4.41 (spt, J=6.7 Hz, 1H), 1.47 (d, J=6.6 Hz, 6H).

### Step 1-4: General Procedure for Preparation of 4-(4-fluorophenyl)-1-isopropyl-5-(tributylstannyl)-1H-imidazole (Compound A7)

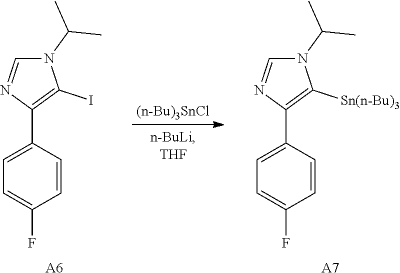

To a solution of compound A6 (65 g, 196.89 mmol, 1 eq) in THE (2000 mL) was added n-BuLi (2.5 M, 102.38 mL, 1.3 eq) dropwise at −70 °C under N₂, after stirring at −70 °C for 15 min, tributyl(chloro)stannane (96.13 g, 295.33 mmol, 79.45 mL, 1.5 eq) was added dropwise at −70 °C, the mixture was stirred at −40-−50 °C for 0.5 h. LCMS showed compound A6 was consumed and desired mass was detected. It was poured into saturated KF aqueous (2 L), EA (2 L) was added, the organic layer was washed with saturated NH₄Cl aqueous (1 L) and brine (1 L), dried over Na₂SO₄, filtered and concentrated in vacuum. The crude was purified by column chromatography (SiO₂, PE:EA 50:1~1:1). Compound A7 (40 g, 72.27 mmol, 36.71% yield, 89.123% purity) was obtained as a yellow oil, which was confirmed by LCMS. LCMS: Retention time: 1.007 min, (M+H)=494.9.

### Step 1-6: General Procedure for Preparation of ethyl 1-((2-(trimethylsilyl)ethoxy)methyl)-1H-imidazole-5-carboxylate (Compound A9)

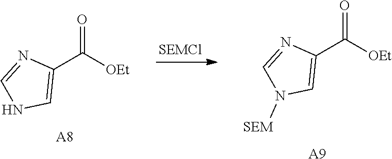

To a suspend of NaH (42.81 g, 1.07 mol, 60% purity, 1.5 eq) in THF (1 L) was added ethyl ethyl 1H-imidazole-5-carboxylate (compound A8) (100 g, 713.57 mmol, 1 eq) in portions at 0 °C, it was stirred at 20 °C for 0.5 h, SEM-C₁ (178.45 g, 1.07 mol, 189.44 mL, 1.5 eq) was added dropwise at 0 °C, it was stirred at 25 °C for 16 h. TLC (PE:EA 1:1) showed compound A8 (Rf=0.1) was consumed completely and a new spot (Rf=0.3) was detected. It was poured into saturated aq. NH₄Cl (1 L) aqueous, and extracted with ethyl acetate (1 L), the organic layer was washed with brine (1 L), dried over Na₂SO₄, filtered and concentrated in vacuum. The crude was purified by column chromatography (SiO₂, PE:EA 10:1~0:1). Compound A9 (136 g, 502.96 mmol, 70.48% yield) was obtained as a yellow solid, which was confirmed by ¹H-NMR. ¹H-NMR: (400 MHz, CHLOROFORM-d) 6=7.74 (d, J=1.3 Hz, 1H), 7.65 (d, J=1.1 Hz, 1H), 5.32 (s, 2H), 4.43-4.36 (m, 2H), 3.54-3.44 (m, 2H), 1.44-1.38 (m, 3H), 1.29-1.26 (m, 2H), 0.96-0.89 (m, 3H), 0.02~−0.02 (m, 9H)

### Step 1-7: General Procedure for Preparation of ethyl 2-bromo-1-((2-(trimethylsilyl)ethoxy)methyl)-1H-imidazole-4-carboxylate (Compound A10)

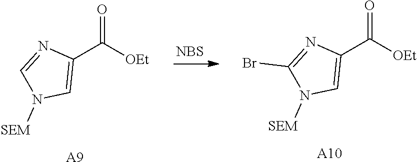

To a solution of ethyl compound A9 (116 g, 429.00 mmol, 1 eq) in CHCl₃ (1 L) was added NBS (83.99 g, 471.89 mmol, 1.1 eq) and AIBN (8.45 g, 51.48 mmol, 0.12 eq), it was stirred at 65 °C for 4 h. TLC (PE:EA 3:1) showed compound A9 (Rf=0.1) was consumed and a new peak (Rf=0.4) was detected. The reaction mixture was quenched with brine (1 L), the organic layer was dried over Na₂SO₄, filtered and concentrated in vacuum. The crude was purified by column chromatography (SiO₂, PE:EA 20:1~2:1). Compound A10 (80 g, 229.03 mmol, 53.39% yield) was obtained as a yellow solid, which was confirmed by ¹H-NMR. ¹H-NMR: (400 MHz, DMSO-d₆) δ=8.30-8.24 (m, 1H), 8.20 (s, 1H), 5.38 (s, 2H), 4.30-4.22 (m, 2H), 3.63-3.53 (m, 2H), 1.33-1.28 (m, 3H), 0.89 (t, J=7.9 Hz, 2H), 0.02~−0.03 (m, 9H).

### Step 1-8: General Procedure for Preparation of ethyl 5′-(4-fluorophenyl)-3′-isopropyl-1-((2-(trimethylsilyl)ethoxy)methyl)-1H,3′H-[2,4′-biimidazole]-4-carboxylate (Compound A11)

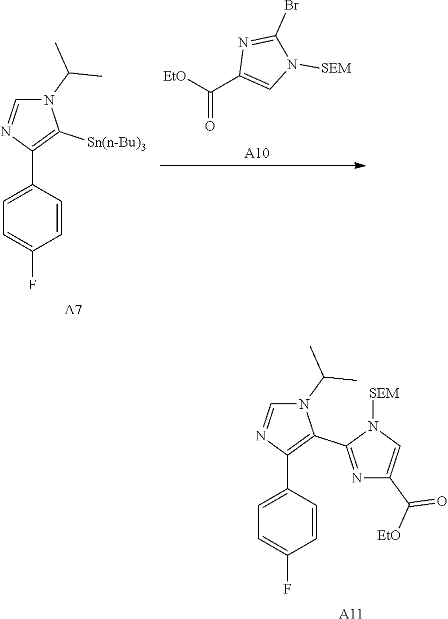

To a solution of compound A7 (10 g, 20.27 mmol, 1 eq) and compound A10 (10.62 g, 30.41 mmol, 1.5 eq) in toluene (100 mL) was added [2-(2-aminophenyl)phenyl]-chloro-palladium; bis(1-adamantyl)-butyl-phosphane (4.07 g, 6.08 mmol, 0.3 eq) at 25 T, it was charged with N₂ three times and stirred at 110 °C for 16 h. TLC (PE:EA 1:1) showed compound A7 (Rf=0.4) was consumed and a new spot (Rf=0.3) was detected. LCMS showed compound A7 was consumed and desired mass was detected. It was filtered and poured into water (300 ml), the pH of the mixture was adjusted to 3 by IN HCl, the organic layer was washed with saturated NaHCO₃ aqueous (300 ml) and brine (300 ml), filtered, dried over Na₂SO₄ and concentrated in vacuum. The crude was purified by column chromatography (SiO₂, PE:EA 100:1~1:2). Compound A11 (20 g, 25.00 mmol, 41.11% yield, 59.089% purity) was obtained as a yellow oil, which was confirmed by LCMS. LCMS: Retention time: 0.927 min, (M+H)=473.3.

### Step 1-9: General Procedure for Preparation of 5′-(4-fluorophenyl)-3′-isopropyl-1-((2-(trimethylsilyl)ethoxy)methyl)-1H,3′H-[2,4′-biimidazole]-4-carboxylic acid (Compound A12)

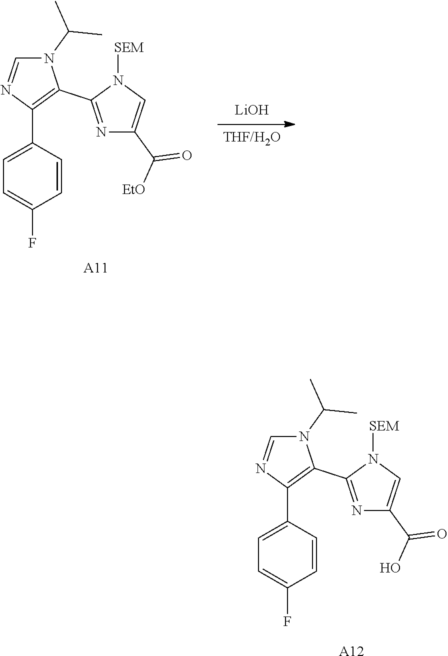 [SMILES: CCOC(=O)c1cn(COCC[Si](C)(C)C)c(-c2c(-c3ccc(F)cc3)ncn2C(C)C)n1 ; "LiOH" ; "A11" ; CC(C)n1cnc(-c2ccc(F)cc2)c1-c1nc(C(=O)O)cn1COCC[Si](C)(C)C ; "A12"]

To a solution of compound A11 (20 g, 42.32 mmol, 1 eq) in THF (60 mL), MeOH (60 mL) and H₂O (60 mL) was added LiOH·H₂O (17.76 g, 423.17 mmol, 10 eq), it was stirred at 25 °C for 2 h. TLC (PE:EA 1:1) showed compound A11 was consumed and a new spot (Rf=0) was detected. The crude was concentrated in vacuum to remove THF and MeOH, water 0.5 L was added, the pH of the mixture was adjusted to 6 by IN HCl, ethyl acetate (1 L) was added, the organic layer was washed with brine (0.5 L), dried over Na₂SO₄ and concentrated in vacuum. The crude was triturated with petroleum ether (70 ml) and MTBE (25 m 1), the mixture was filtered and the cake was washed with petroleum ether (20 ml) and dried in vacuum, the cake was purified by reversed-phase HPLC (1% TFA condition). Compound A12 (11.5 g, 25.01 mmol, 59.10% yield, 96.686% purity) was obtained as an off-white solid, which was confirmed by LCMS and ¹H-NMR. LCMS: Retention time: 0.877 min, (M+H)=445.2. ¹H-NMR: (400 MHz, DMSO-d₆) δ=13.40-11.99 (m, 1H), 8.34 (s, 1H), 8.24 (s, 1H), 7.41-7.36 (m, 2H), 7.27-7.20 (m, 2H), 5.12-4.96 (m, 2H), 4.22-4.15 (m, 1H), 3.33 (br d, J=4.5 Hz, 2H), 1.58-1.43 (m, 6H), 0.68 (br dd, J=5.0, 8.8 Hz, 2H), 0.03~−0.02 (m, 9H),

### Step 1-10: General Procedure for Preparation of 5′-(4-fluorophenyl)-3′-isopropyl-N-(4-(4-methylpiperazin-1-yl)phenyl)-1-((2-(trimethylsilyl)ethoxy)methyl)-1H,3′H-[2,4′-biimidazole]-4-carboxamide (Compound A14)

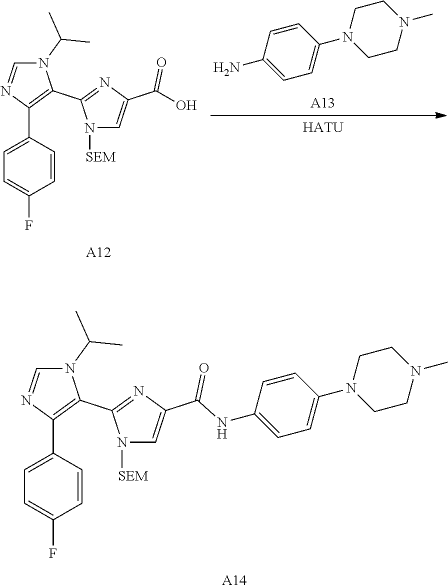

To a solution of Compound A12 (3.5 g, 7.87 mmol, 1 eq) and 4-(4-methylpiperazin-1-yl)aniline (Compound A13) (1.81 g, 9.45 mmol, 1.2 eq) in DMF (35 mL) was added HATU (4.49 g, 11.81 mmol, 1.5 eq) and DIEA (3.05 g, 23.62 mmol, 4.11 mL, 3.0 eq), it was stirred at 25 °C for 2 h. LCMS showed Compound A12 was consumed and desired mass was detected. It was combined with other two batches for work-up, the mixture was poured into water (300 ml), ethyl acetate (300 ml) was added, the organic layer was washed with brine (200 ml×3), dried over Na₂SO₄ and concentrated in vacuum. The crude was used in the next step directly. Compound A14 (9 g, crude) was obtained as a yellow solid, which was confirmed by LCMS. LCMS: Retention time: 0.801 min, (M+H)=618.4.

### Step 1-11: General Procedure for Preparation of 5′-(4-fluorophenyl)-3′-isopropyl-N-(4-(4-methylpiperazin-1-yl)phenyl)-1H,3′H-[2,4′-biimidazole]-4-carboxamide (Compound 112)

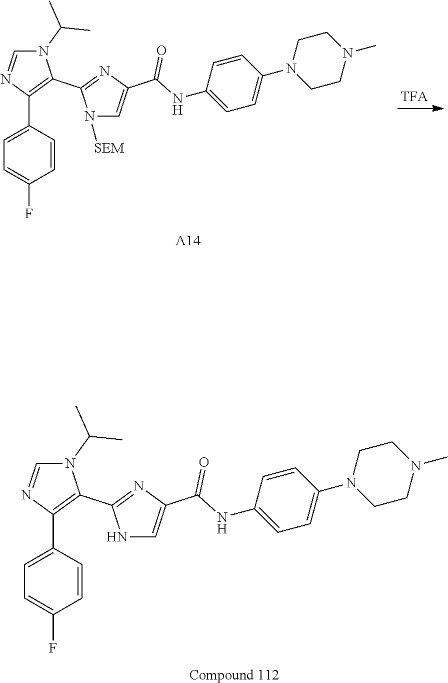 [SMILES: CC(C)n1cnc(-c2ccc(F)cc2)c1-c1nc(C(=O)Nc2ccc(N3CCN(C)CC3)cc2)cn1COCC[Si](C)(C)C ; "A14" ; CC(C)n1cnc(-c2ccc(F)cc2)c1-c1nc(C(=O)Nc2ccc(N3CCN(C)CC3)cc2)c[nH]1 ; "Compound 112"]

To a solution of compound A14 (9 g, 14.57 mmol, 1 eq) in DCM (10 mL) was added TFA (90 mL), it was stirred at 25 °C for 2 h. LCMS showed compound A14 was consumed and desired mass was detected. It was concentrated in vacuum. The crude was purified by Prep-HPLC (column: Kromasil Eternity XT 250×80 mm×10 um; mobile phase: [water (0.05% ammonia hydroxide v/v)-ACN]; B %: 30%-60%, 15 min) and lyophylizated. Compound 112 (4.42 g, 8.45 mmol, 58.03% yield, 93.215% purity) was obtained, which was confirmed by ¹H-NMR, and LCMS. LCMS: Retention time: 0.611 min, (M±H)=488.3. ¹H-NMR: (400 MHz, DMSO-d₆) δ=13.64-12.60 (m, 1H), 9.67 (br s, 1H), 8.03 (s, 1H), 7.93 (s, 1H), 7.65 (br d, J=8.9 Hz, 2H), 7.43 (dd, J=5.7, 8.6 Hz, 2H), 7.20-7.06 (m, 2H), 6.89 (d, J=9.0 Hz, 2H), 4.26 (td, J=6.7, 13.4 Hz, 1H), 3.16-3.02 (m, 4H), 2.47-2.41 (m, 4H), 2.22 (s, 3H), 1.39 (d, J=6.7 Hz, 6H).

### Example 2: New Synthesis Route 1 for Compound 112

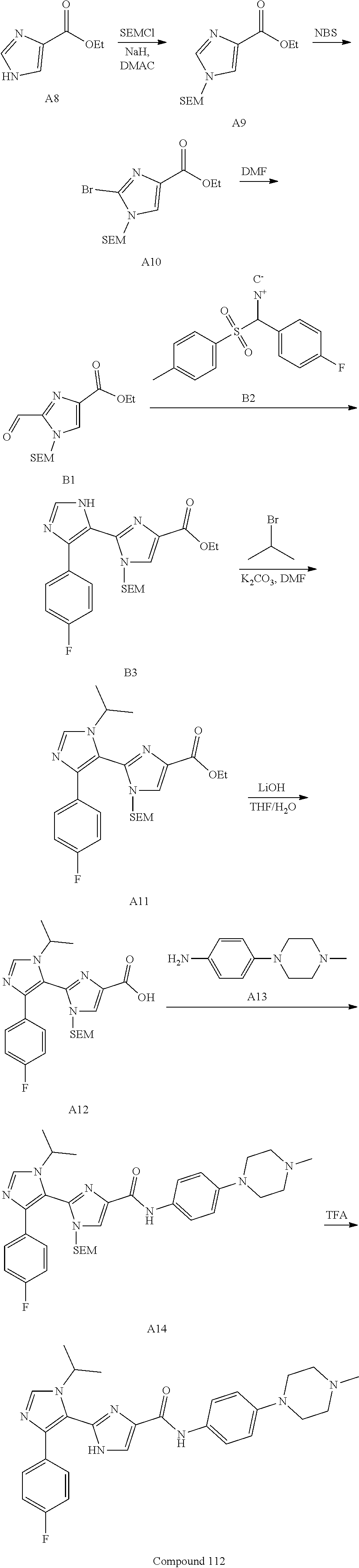

### Step 2-1: General Procedure for Preparation of ethyl 1-((2-(trimethylsilyl)ethoxy)methyl)-1H-imidazole-5-carboxylate (Compound A9)

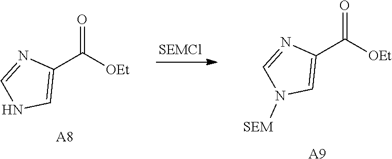

To a suspend of NaH (42.81 g, 1.07 mol, 60% purity, 1.5 eq) in THF (1 L) was added ethyl ethyl 1H-imidazole-5-carboxylate (compound A8) (100 g, 713.57 mmol, 1 eq) in portions at 0 °C, it was stirred at 20 °C for 0.5 h, SEM-Cl (178.45 g, 1.07 mol, 189.44 mL, 1.5 eq) was added dropwise at 0 °C, it was stirred at 25 °C for 16 h. TLC (PE:EA 1:1) showed compound A8 (Rf=0.1) was consumed completely and a new spot (Rf=0.3) was detected. It was poured into saturated NH₄Cl (1 L) aqueous, and extracted with ethyl acetate (1 L), the organic layer was washed with brine (1 L), dried over Na₂SO₄, filtered and concentrated in vacuum. The crude was purified by column chromatography (SiO₂, PE:EA 10:1~0:1). Compound A9 (136 g, 502.96 mmol, 70.48% yield) was obtained as a yellow solid, which was confirmed by ¹H-NMR. ¹H-NMR: (400 MHz, CHLOROFORM-d) 6=7.74 (d, J=1.3 Hz, 1H), 7.65 (d, J=1.1 Hz, 1H), 5.32 (s, 2H), 4.43-4.36 (m, 2H), 3.54-3.44 (m, 2H), 1.44-1.38 (m, 3H), 1.29-1.26 (m, 2H), 0.96-0.89 (m, 3H), 0.02~−0.02 (m, 9H)

Further examples of Step 2-1: The ethyl ester group of Compound A8 could be replaced with methyl ester, n-PrOH ester, i-PrOH ester, t-BuOH ester, phenyl ester, benzyl ester, p-methoxybenzyl ester, etc.

The protection group SEM could be replaced by commercially available protection group, eg., methyloxycarbonyl, ethyloxycarbonyl, Cbz (benzyloxycarbonyl), Boc (tert-butoxycarbonyl), Fmoc (9-fluorenylmethyloxycarbonyl), Alloc (allyloxycarbonyl), Teoc (2-(trimethylsilyl)ethyloxycarbonyl), Troc (2,2,2-trichloroethoxycarbonyl), Tos (para-toluenesulfonyl), Tfa (2,2,2-trifluoroacetyl), Trt (trityl), Dmb (2,4-dimethocybenzyl), Pmb (p-Methoxybenzyl), Bn (benzyl).

### Step 2-2: General Procedure for Preparation of ethyl 2-bromo-1-((2-(trimethylsilyl)ethoxy)methyl)-1H-imidazole-4-carboxylate (Compound A10)

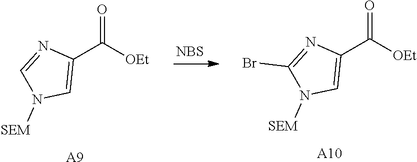

To a solution of ethyl compound A9 (116 g, 429.00 mmol, 1 eq) in CHCl₃ (1 L) was added NBS (83.99 g, 471.89 mmol, 1.1 eq) and AIBN (8.45 g, 51.48 mmol, 0.12 eq), it was stirred at 65 °C for 4 h. TLC (PE:EA 3:1) showed compound A9 (Rf=0.1) was consumed and a new peak (Rf=0.4) was detected. The reaction mixture was quenched with brine (1 L), the organic layer was dried over Na₂SO₄, filtered and concentrated in vacuum. The crude was purified by column chromatography (SiO₂, PE:EA 20:1~2:1). Compound A10 (80 g, 229.03 mmol, 53.39% yield) was obtained as a yellow solid, which was confirmed by ¹H-NMR. ¹H-NMR: (400 MHz, DMSO-d₆) δ=8.30-8.24 (m, 1H), 8.20 (s, 1H), 5.38 (s, 2H), 4.30-4.22 (m, 2H), 3.63-3.53 (m, 2H), 1.33-1.28 (m, 3H), 0.89 (t, J=7.9 Hz, 2H), 0.02~−0.03 (m, 9H).

### Step 2-3: General Procedure for Preparation of ethyl 2-formyl-1-((2-(trimethylsilyl)ethoxy)methyl)-1H-imidazole-4-carboxylate (Compound B1)

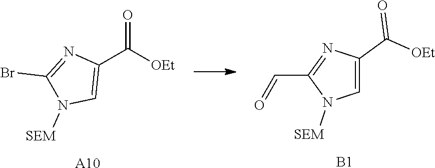

To a solution of Compound A10 (28 g, 80.16 mmol, 1 eq) in THF (300 mL) was added i-PrMgCl (2 M, 120.24 mL, 3 eq) at −40′C. The mixture was stirred at −40 °C for 10 min. To the mixture was added DMF (35.16 g, 480.97 mmol, 37.01 mL, 6 eq) at −70 °C. The mixture was stirred at 20 °C for 1 hrs. HPLC was showed Compound A10 was consumed. LCMS was showed desired mass was detected. The mixture was poured to IN HCl (500 mL). The mixture was extracted with EA (300 mL×2). The combined organic layers were dried over Na₂SO₄, filtered and concentrated under reduced pressure to give a residue. The residue was purified by column chromatography on silica gel (PE:EA=10:1~3:1). Compound B1 (15 g, 50.27 mmol, 62.71% yield) was obtained as yellow oil, which was checked with LCMS and ¹H-NMR. LCMS: Retention time: 0.955 min, (M+H)=299.2. ¹H-NMR: (400 MHz, CHLOROFORM-d) 8=8.03-7.95 (m, 1H), 5.80 (s, 2H), 4.47-4.41 (m, 2H), 3.63-3.57 (m, 2H), 1.46-1.40 (m, 3H), 0.99-0.93 (m, 2H), 0.00 (s, 8H)

In some case, the strong base i-PrMgCl for bromine-metal exchange reaction could be replaced with commercially available strong base, e.g. LDA, n-BuLi, t-BuLi, LiHMDS, KHMDS, i-PrMgBr, MeMgCl, MeMgBr, EtMgCl, EtMgBr, etc.

DMF could be replaced with other formylation reagent, e.g., N,N-diethylformamide, etc.

### Step 2-4: General Procedure for Preparation of ethyl 5′-(4-fluorophenyl)-1-((2-(trimethylsilyl)ethoxy)methyl)-1H,3′H-[2,4′-biimidazole]-4-carboxylate (Compound B3)

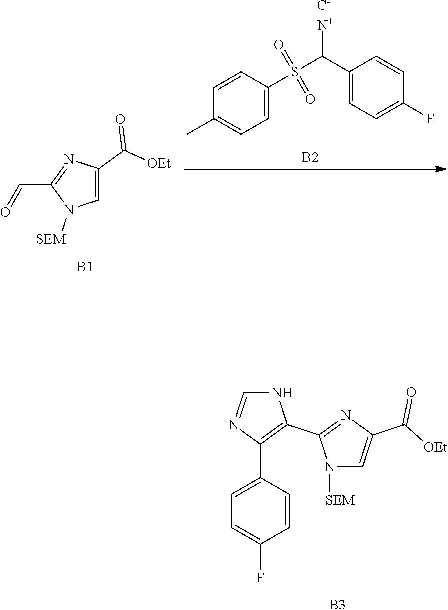

To a mixture of Compound B1 (12.5 g, 41.89 mmol, 1 eq) in THF (200 mL) was added NH₃·H₂O (22.73 g, 162.11 mmol, 24.97 mL, 25% purity, 3.87 eq). The mixture was stirred at 20 °C for 4 hours. To the mixture was added Compound B2 (14.54 g, 50.27 mmol, 1.2 eq) and DIEA (16.24 g, 125.67 mmol, 21.89 mL, 3 eq) at 20 TC. The mixture was stirred at 20 °C for 2 hrs. TLC (PE:EA=0:1) showed Compound B1 was consumed and a new spot (Rf=0.3) was detected. The residue was poured into water (150 mL) and extracted with ethyl acetate (150 mL×3). The combined organic phase was washed with brine (200 mL×1), dried with anhydrous Na₂SO₄, filtered and concentrated in vacuum. The crude product was purified by column chromatography on silica gel (PE:EA=1:1~0:1). Compound B3 (10 g, 22.51 mmol, 53.74% yield, 96.914% purity) was obtained as yellow solid, which was checked with LCMS. LCMS: Retention time: 0.799 min, (M+H)=431.2.

### Step 2-5: General Procedure for Preparation of ethyl 5′-(4-fluorophenyl)-3′-isopropyl-1-((2-(trimethylsilyl)ethoxy)methyl)-1H,3′H-[2,4′-biimidazole]-4-carboxylate (Compound A11)

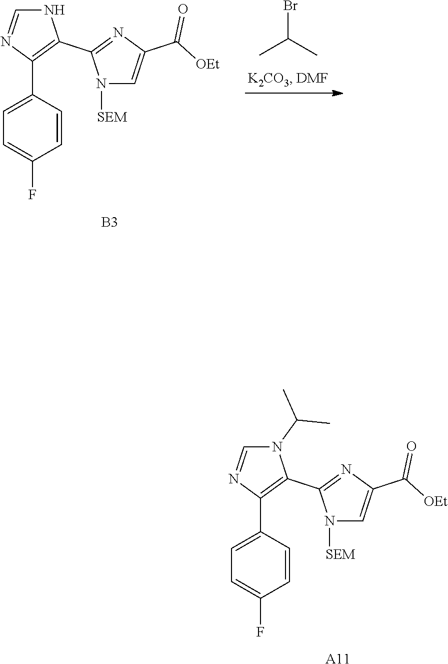

To a mixture of Compound B3 (10 g, 23.23 mmol, 1 eq) in DMF (60 mL) was added K₂CO₃ (9.63 g, 69.68 mmol, 3 eq) and 2-bromopropane (8.57 g, 69.68 mmol, 6.54 mL, 3 eq). The mixture was stirred at 60 °C for 6 hours. LCMS showed Compound B3 was consumed and the desired mass was detected. The residue was poured into ice-water (500 mL) and extracted with ethyl acetate (100 mL×3). The combined organic phase was washed with brine (200 mL×1), dried with anhydrous Na₂SO₄, filtered and concentrated in vacuum. The residue was purified by silica gel chromatography (PE:EA=1:1~1:5). Compound A11 (7.6 g, crude) was obtained as yellow solid, which was checked with LCMS and ¹H-NMR. LCMS: Retention time: 0.826 min, (M+H)=473.3. ¹H-NMR: (400 MHz, DMSO-d₆) δ=8.30 (s, 1H), 8.16-8.09 (m, 1H), 7.27 (br dd, J=5.6, 8.8 Hz, 2H), 7.17-7.06 (m, 2H), 5.03-4.83 (m, 2H), 4.29 (br d, J=6.4 Hz, 2H), 4.06 (br s, 1H), 3.28-3.18 (m, 2H), 1.39 (br s, 6H), 1.32 (t, J=7.2 Hz, 3H), 0.61-0.50 (m, 2H), −0.07~−0.14 (m, 9H)

### Step 2-6: General Procedure for Preparation of 5′-(4-fluorophenyl)-3′-isopropyl-1-((2-(trimethylsilyl)ethoxy)methyl)-1H,3′H-[2,4′-biimidazole]-4-carboxylic acid (Compound A12)

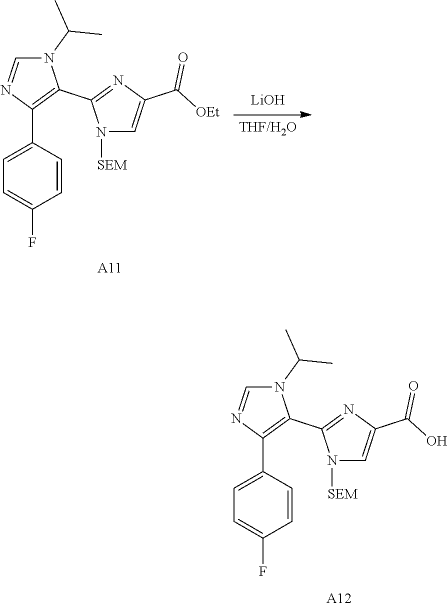 [SMILES: CCOC(=O)c1cn(COCC[Si](C)(C)C)c(-c2c(-c3ccc(F)cc3)ncn2C(C)C)n1 ; "LiOH" ; "A11" ; CC(C)n1cnc(-c2ccc(F)cc2)c1-c1nc(C(=O)O)cn1COCC[Si](C)(C)C ; "A12"]

To a mixture of Compound A11 (7.6 g, 16.08 mmol, 1 eq) in THF (80 mL) and H₂O (16 mL) was added LiOH (2.70 g, 112.56 mmol, 7 eq). The mixture was stirred at 20 °C for 3 hours. LCMS showed reactant was consumed, and desired mass was detected. The residue was acidified with the aqueous 1 N HCl and the resulting mixture was extracted with ethyl acetate (100 mL×3). The combined organic phase was washed with brine (150 mL), dried with anhydrous Na₂SO₄, filtered and concentrated in vacuum. The crude compound was used into the next step without further purification. Compound A12 (6.1 g, crude) was obtained as yellow solid, which was checked with LCMS and ¹H-NMR. LCMS: Retention time: 0.763 min, (M+H)=445.3. ¹H-NMR: (400 MHz, DMSO-d₆) δ=8.98-8.76 (m, 1H), 8.27 (s, 1H), 7.42-7.16 (m, 4H), 5.00 (br s, 2H), 4.16 (td, J=6.8, 13.2 Hz, 1H), 3.21 (br s, 2H), 1.43 (br d, J=6.8 Hz, 6H), 0.53 (br d, J=3.2 Hz, 2H), −0.04~−0.15 (m, 9H)

### General Procedure for Preparation of 5′-(4-fluorophenyl)-3′-isopropyl-N-(4-(4-methylpiperazin-1-yl)phenyl)-1-((2-(trimethylsilyl)ethoxy)methyl)-1H,3′H-[2,4′-biimidazole]-4-carboxamide (Compound A14)

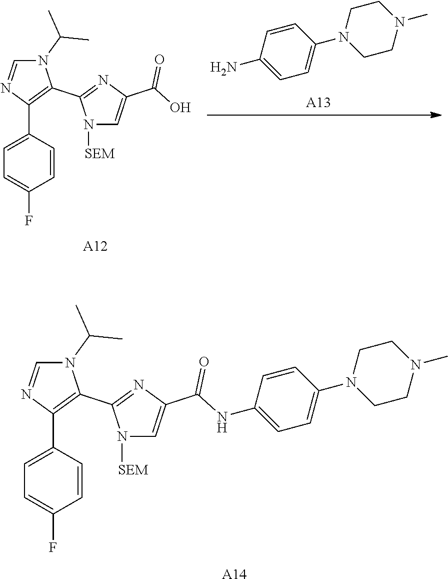

To a mixture of Compound A12 (6.5 g, 14.62 mmol, 1 eq) and Compound A13 (4.19 g, 21.93 mmol, 1.5 eq) in DMF (50 mL) was added HATU (8.34 g, 21.93 mmol, 1.5 eq) and DIEA (5.67 g, 43.86 mmol, 7.64 mL, 3 eq). The mixture was stirred at 20 TC for 3 hours. LCMS showed Compound A12 was consumed and desired mass was detected. The residue was poured into water (500 mL) and extracted with ethyl acetate (150 mL×3). The combined organic phase was washed with brine (200 mL), dried with anhydrous Na₂SO₄, filtered and concentrated in vacuum. The crude compound was used into the next step without further purification. Compound A14 (9 g, crude) was obtained as brown oil, which was checked with LCMS. LCMS: Retention time: 0.753 min, (M+H)=618.4.

### Step 2-8: General Procedure for Preparation of 5′-(4-fluorophenyl)-3′-isopropyl-N-(4-(4-methylpiperazin-1-yl)phenyl)-1H,3′H-[2,4′-biimidazole]-4-carboxamide (Compound 112)

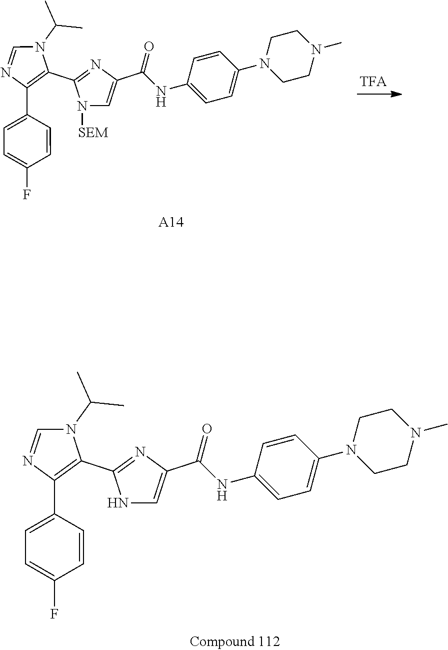 [SMILES: CC(C)n1cnc(-c2ccc(F)cc2)c1-c1nc(C(=O)Nc2ccc(N3CCN(C)CC3)cc2)cn1COCC[Si](C)(C)C ; "A14" ; CC(C)n1cnc(-c2ccc(F)cc2)c1-c1nc(C(=O)Nc2ccc(N3CCN(C)CC3)cc2)c[nH]1]

To a solution of compound A14 (9 g, 14.57 mmol, 1 eq) in DCM (10 mL) was added TFA (90 mL), it was stirred at 25 °C for 2 h. LCMS showed compound A14 was consumed and desired mass was detected. It was concentrated in vacuum. The crude was purified by Prep-HPLC (column: Kromasil Eternity XT 250×80 mm×10 um; mobile phase: [water (0.05% ammonia hydroxide v/v)-ACN]; B %: 30%-60%, 15 min) and lyophylizated. Compound 112 (4.42 g, 8.45 mmol, 58.03% yield, 93.215% purity) was obtained, which was confirmed by ¹H-NMR and LCMS. LCMS: Retention time: 0.611 min, (M+H)=488.3. ¹H-NMR: (400 MHz, DMSO-d₆) δ=13.64-12.60 (m, 1H), 9.67 (br s, 1H), 8.03 (s, 1H), 7.93 (s, 1H), 7.65 (br d, J=8.9 Hz, 2H), 7.43 (dd, J=5.7, 8.6 Hz, 2H), 7.20-7.06 (m, 2H), 6.89 (d, J=9.0 Hz, 2H), 4.26 (td, J=6.7, 13.4 Hz, 1H), 3.16-3.02 (m, 4H), 2.47-2.41 (m, 4H), 2.22 (s, 3H), 1.39 (d, J-6.7 Hz, 6H).

### Example 3: New Synthesis Route 2 for Compound 112

Imidazole ring of Compound A8 was protected by SEM group, and then was brominated at C-2 position to afford Compound A10. Bromine-magnesium exchange with Grignard reagent followed by reaction with DMF introduced the 2-formyl group. A isonitrile chemistry reaction with Compound B2 built the imidazole ring in one step to afford Compound A11. Hydrolysis of the ester group and condensation reaction with Compound A13 gave Compound A14. Final compound was obtained after de-protection of the SEM group.

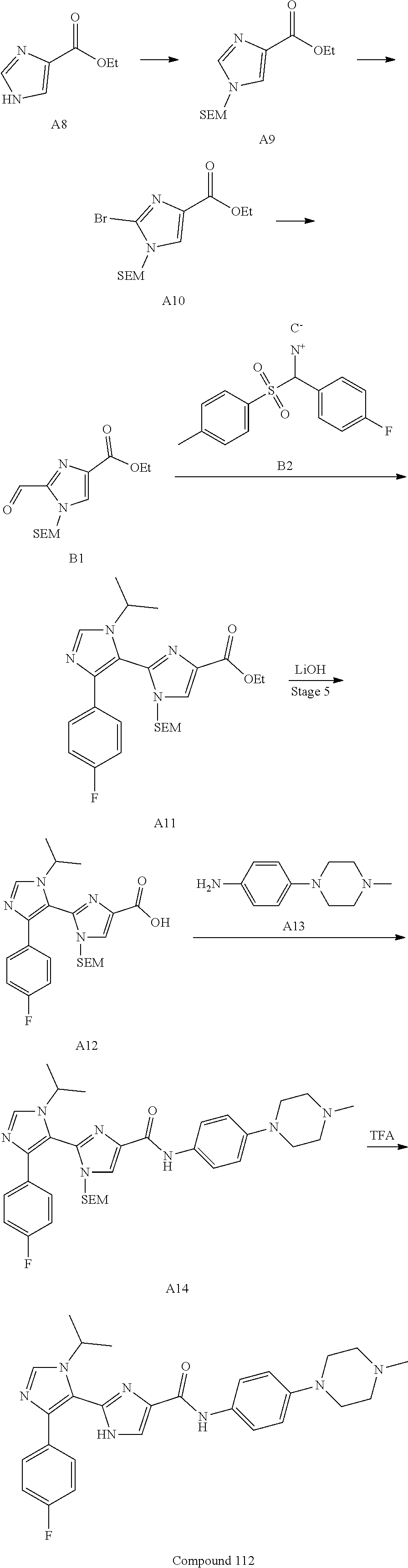

### Step 3-1: General Procedure for Preparation of ethyl 1-((2-(trimethylsilyl)ethoxy)methyl)-1H-imidazole-5-carboxylate (Compound A9)

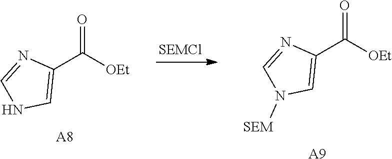

To a suspend of NaH (42.81 g, 1.07 mol, 60% purity, 1.5 eq) in THF (1 L) was added ethyl ethyl 1H-imidazole-5-carboxylate (compound A8) (100 g, 713.57 mmol, 1 eq) in portions at 0 °C, it was stirred at 20 °C for 0.5 h, SEM-Cl (178.45 g, 1.07 mol, 189.44 mL, 1.5 eq) was added dropwise at 0 °C, it was stirred at 25 °C for 16 h. TLC (PE:EA 1:1) showed compound A8 (Rf=0.1) was consumed completely and a new spot (Rf=0.3) was detected. It was poured into saturated NH₄Cl (1 L) aqueous, and extracted with ethyl acetate (1 L), the organic layer was washed with brine (1 L), dried over Na₂SO₄, filtered and concentrated in vacuum. The crude was purified by column chromatography (SiO₂, PE:EA 10:1-0:1). Compound A9 (136 g, 502.96 mmol, 70.48% yield) was obtained as a yellow solid, which was confirmed by ¹H-NMR. ¹H-NMR: (400 MHz, CHLOROFORM-d) 6-7.74 (d, J=1.3 Hz, 1H), 7.65 (d, J=1.1 Hz, 1H), 5.32 (s, 2H), 4.43-4.36 (m, 2H), 3.54-3.44 (m, 2H), 1.44-1.38 (m, 3H), 1.29-1.26 (m, 2H), 0.96-0.89 (m, 3H), 0.02~−0.02 (m, 9H)

In some case, the ethyl ester group of Compound A8 could be replaced with methyl ester, n-PrOH ester, i-PrOH ester, t-BuOH ester, phenyl ester, benzyl ester, p-methoxybenzyl ester, etc.

The protection group SEM could be replaced by commercially available protection group, eg., methyloxycarbonyl, ethyloxycarbonyl, Cbz (benzyloxycarbonyl), Boc (tert-butoxycarbonyl), Fmoc (9-fluorenylmethyloxycarbonyl), Alloc (allyloxycarbonyl), Teoc (2-(trimethylsilyl)ethyloxycarbonyl), Troc (2,2,2-trichloroethoxycarbonyl), Tos (para-toluenesulfonyl), Tfa (2,2,2-trifluoroacetyl), Trt (trityl), Dmb (2,4-dimethocybenzyl), Pmb (p-Methoxybenzyl), Bn (benzyl).

### Step 3-2: General Procedure for Preparation of ethyl 2-bromo-1-((2-(trimethylsilyl)ethoxy)methyl)-1H-imidazole-4-carboxylate (Compound A10)

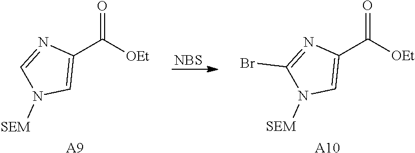

To a solution of ethyl compound A9 (116 g, 429.00 mmol, 1 eq) in CHCl₃ (1 L) was added NBS (83.99 g, 471.89 mmol, 1.1 eq) and AIBN (8.45 g, 51.48 mmol, 0.12 eq), it was stirred at 65 T for 4 h. TLC (PE:EA 3:1) showed compound A9 (Rf=0.1) was consumed and a new peak (Rf=0.4) was detected. The reaction mixture was quenched with brine (1 L), the organic layer was dried over Na₂SO₄, filtered and concentrated in vacuum. The crude was purified by column chromatography (SiO₂, PE:EA 20:1~2:1). Compound A10 (80 g, 229.03 mmol, 53.39% yield) was obtained as a yellow solid, which was confirmed by ¹H-NMR. ¹H-NMR: (400 MHz, DMSO-d₆) δ=8.30-8.24 (m, 1H), 8.20 (s, 1H), 5.38 (s, 2H), 4.30-4.22 (m, 2H), 3.63-3.53 (m, 2H), 1.33-1.28 (m, 3H), 0.89 (t, J=7.9 Hz, 2H), 0.02~−0.03 (m, 9H).

### Step 3-3: General Procedure for Preparation of ethyl 2-formyl-1-((2-(trimethylsilyl)ethoxy)methyl)-1H-imidazole-4-carboxylate (Compound B1)

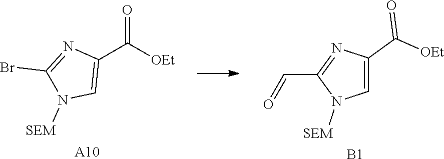

To a solution of Compound A10 (28 g, 80.16 mmol, 1 eq) in THF (300 mL) was added i-PrMgCl (2 M, 120.24 mL, 3 eq) at −40 T. The mixture was stirred at −40 T for 10 min. To the mixture was added DMF (35.16 g, 480.97 mmol, 37.01 mL, 6 eq) at −70 T. The mixture was stirred at 20 °C for 1 hrs. HPLC was showed Compound A10 was consumed. LCMS was showed desired mass was detected. The mixture was poured to IN HCl (500 mL). The mixture was extracted with EA (300 mL×2). The combined organic layers were dried over Na2SO4, filtered and concentrated under reduced pressure to give a residue. The residue was purified by column chromatography on silica gel (PE:EA=10:1-3:1). Compound B1 (15 g, 50.27 mmol, 62.71% yield) was obtained as yellow oil, which was checked with LCMS and ¹H-NMR. LCMS: Retention time: 0.955 min, (M+H)=299.2. ¹H-NMR: (400 MHz, CHLOROFORM-d) 6=8.03-7.95 (m, 1H), 5.80 (s, 2H), 4.47-4.41 (m, 2H), 3.63-3.57 (m, 2H), 1.46-1.40 (m, 3H), 0.99-0.93 (m, 2H), 0.00 (s, 8H).

In some cases, the strong base i-PrMgCl for bromine-metal exchange reaction could be replaced with commercially available strong base, eg. LDA, n-BuLi, t-BuLi, LiHMDS, KHMDS, i-PrMgBr, MeMgCl, MeMgBr, EtMgCl, EtMgBr, etc.

In some cases, DMF could be replaced with other formylation reagent, eg., N,N-diethylformamide, etc.

### Step 3-4: General Procedure for Preparation of ethyl 5′-(4-fluorophenyl)-3′-isopropyl-1-((2-(trimethylsilyl)ethoxy)methyl)-1H,3′H-[2,4′-biimidazole]-4-carboxylate (Compound A11)

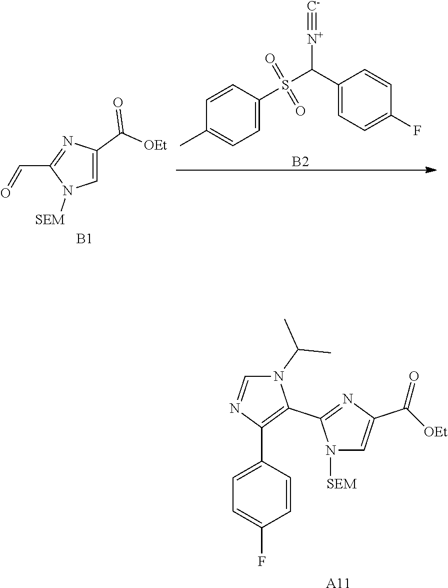

To a mixture of Compound B1 (5 g, 16.76 mmol, 1 eq) in THF (20 mL) was added i-PrNH₂ (3.96 g, 67.02 mmol, 5.76 mL, 4 eq). The mixture was stirred at 20 °C for 4 hours. To the mixture was added Compound B2 (5.82 g, 20.11 mmol, 1.2 eq) and DIEA (6.50 g, 50.27 mmol, 8.76 mL, 3 eq) at 20 °C. The mixture was stirred at 20 °C for 1 hr. LCMS showed 81.7% of Compound A11 formed. The reaction mixture was concentrated under reduced pressure to remove solvent. The residue was purified by flash silica gel chromatography (ISCO®; 80 g SepaFlash® Silica Flash Column, Eluent of 0~100% Ethylacetate/Petroleum ethergradient @120 mL/min). Compound A11 (6.17 g, 13.05 mmol, 77.91% yield, 100% purity) was obtained as a yellow oil.

### Step 3-5: General Procedure for Preparation of 5′-(4-fluorophenyl)-3′-isopropyl-1-((2-(trimethylsilyl)ethoxy)methyl)-1H,3′H-[2,4′-biimidazole]-4-carboxylic acid (Compound A12)

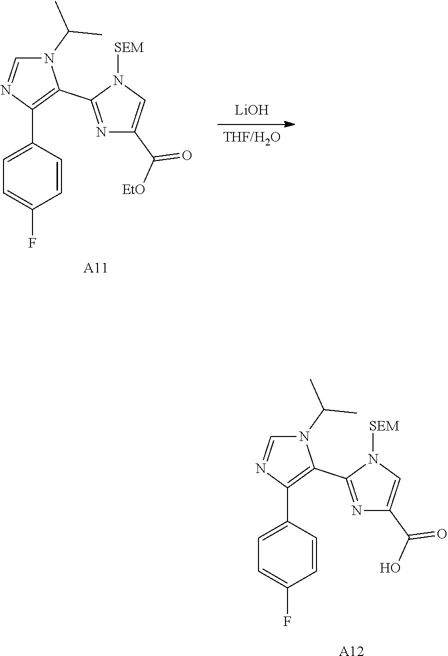 [SMILES: CCOC(=O)c1cn(COCC[Si](C)(C)C)c(-c2c(-c3ccc(F)cc3)ncn2C(C)C)n1 ; "LiOH" ; "A11" ; CC(C)n1cnc(-c2ccc(F)cc2)c1-c1nc(C(=O)O)cn1COCC[Si](C)(C)C ; "A12"]

To a mixture of Compound A11 (7.6 g, 16.08 mmol, 1 eq) in THF (80 mL) and H₂O (16 mL) was added LiOH (2.70 g, 112.56 mmol, 7 eq). The mixture was stirred at 20 °C for 3 hours. LCMS showed reactant was consumed, and desired mass was detected. The residue was acidified with the aqueous 1 M HCl and the resulting mixture was extracted with ethyl acetate (100 mL×3). The combined organic phase was washed with brine (150 mL), dried with anhydrous Na₂SO₄, filtered and concentrated in vacuum. The crude compound was used into the next step without further purification. Compound A12 (6.1 g, crude) was obtained as yellow solid, which was checked with LCMS and ¹H-NMR. LCMS: Retention time: 0.763 min, (M+H)=445.3. ¹H-NMR: (400 MHz, DMSO-d₆) δ=8.98-8.76 (m, 1H), 8.27 (s, 1H), 7.42-7.16 (m, 4H), 5.00 (br s, 2H), 4.16 (td, J=6.8, 13.2 Hz, 1H), 3.21 (br s, 2H), 1.43 (br d, J=6.8 Hz, 6H), 0.53 (br d, J=3.2 Hz, 2H), −0.04~−0.15 (m, 9H) Step 3-6: General Procedure for Preparation of 5′-(4-fluorophenyl)-3′-isopropyl-N-(4-(4-methylpiperazin-1-yl)phenyl)-1-((2-(trimethylsilyl)ethoxy)methyl)-1H,3′H-[2,4′-biimidazole]-4-carboxamide (Compound A14)

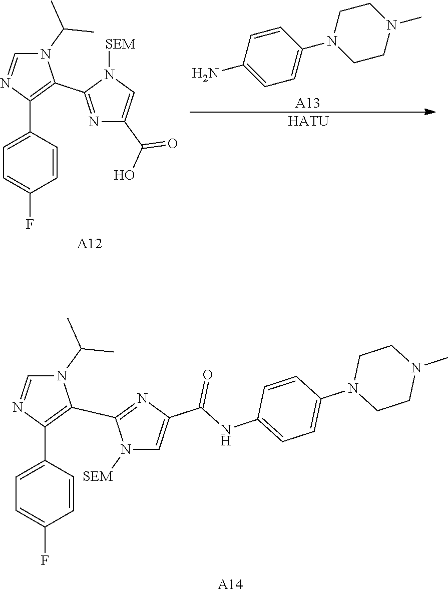

To a mixture of Compound A12 (6.5 g, 14.62 mmol, 1 eq) and Compound A13 (4.19 g, 21.93 mmol, 1.5 eq) in DMF (50 mL) was added HATU (8.34 g, 21.93 mmol, 1.5 eq) and DIEA (5.67 g, 43.86 mmol, 7.64 mL, 3 eq). The mixture was stirred at 20 °C for 3 hours. LCMS showed Compound A12 was consumed and desired mass was detected. The residue was poured into water (500 mL) and extracted with ethyl acetate (150 mL×3). The combined organic phase was washed with brine (200 mL), dried with anhydrous Na₂SO₄, filtered and concentrated in vacuum. The crude compound was used into the next step without further purification. Compound A14 (9 g, crude) was obtained as brown oil, which was checked with LCMS. LCMS: Retention time: 0.753 min, (M+H)=618.4.

### Step 3-7: General Procedure for Preparation of 5′-(4-fluorophenyl)-3′-isopropyl-N-(4-(4-methylpiperazin-1-yl)phenyl)-1H,3′H-[2,4′-biimidazole]-4-carboxamide (Compound 112)

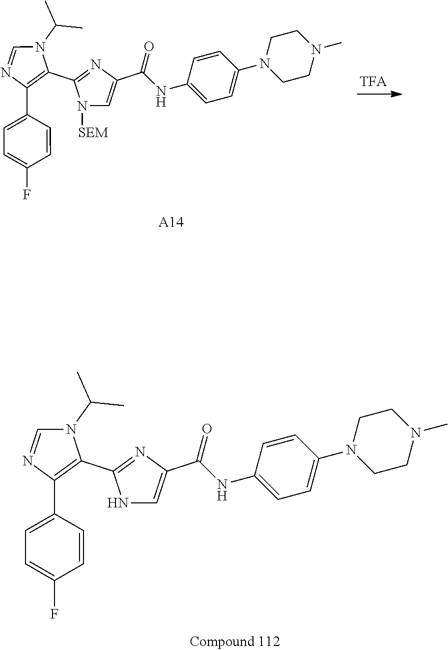 [SMILES: CC(C)n1cnc(-c2ccc(F)cc2)c1-c1nc(C(=O)Nc2ccc(N3CCN(C)CC3)cc2)cn1COCC[Si](C)(C)C ; "A14" ; CC(C)n1cnc(-c2ccc(F)cc2)c1-c1nc(C(=O)Nc2ccc(N3CCN(C)CC3)cc2)c[nH]1]

To a mixture of Compound A14 (9 g, 14.57 mmol, 1 eq) in CH₂Cl₂ (2 mL) was added TFA (30.80 g, 270.12 mmol, 20.00 mL, 18.54 eq). The mixture was stirred at 20 TC for 5 hours. LCMS showed Compound A14 was consumed and the desired mass was detected. The mixture was basified with aqueous NaHCO₃) till pH=8 and the resulting mixture was extracted with ethyl acetate (200 mL×4). The combined organic phase was washed with brine (100 mL), dried with anhydrous Na₂SO₄, filtered and concentrated in vacuum. The residue was purified by reversed-phase HPLC (0.1% NH₃—H₂O). Compound 112 (3.04 g, 6.20 mmol, 42.58% yield, 99.492% purity) was obtained as off-white solid, which was checked with LCMS and ¹H-NMR. LCMS: Retention time: 0.683 min, (M+H)=488.2. ¹H-NMR: (400 MHz, DMSO-d₆) δ=13.14-12.98 (m, 1H), 9.72 (s, 1H), 8.07 (s, 1H), 7.98 (s, 1H), 7.67 (d, J=9.2 Hz, 2H), 7.44-7.34 (m, 2H), 7.14 (t, J=8.8 Hz, 2H), 6.90 (d, J=9.0 Hz, 2H), 4.30-4.18 (m, 1H), 3.13-3.04 (m, 4H), 2.47-2.42 (m, 4H), 2.22 (s, 3H), 1.40 (d, J=6.4 Hz, 6H).

### Example 4: New Synthesis Route 3 for Compound 112

The synthesis from Compound A8 to Compound B1 was same as new route 1. Compound B1 was de-protected, followed by the isonitrile chemistry reaction with Compound B2 to build the imidazole ring in one step. Hydrolysis of the ester group and condensation reaction with Compound A13 gave target compound Compound 112.

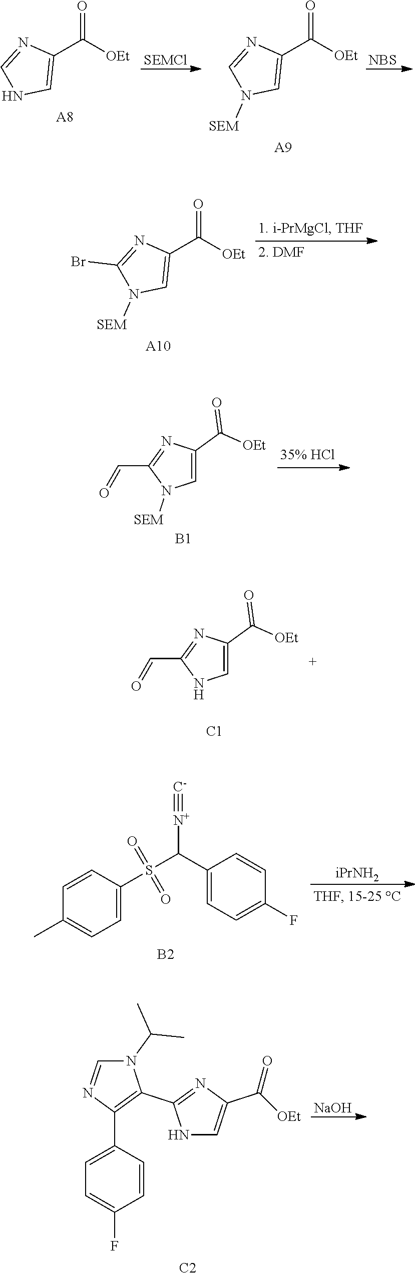

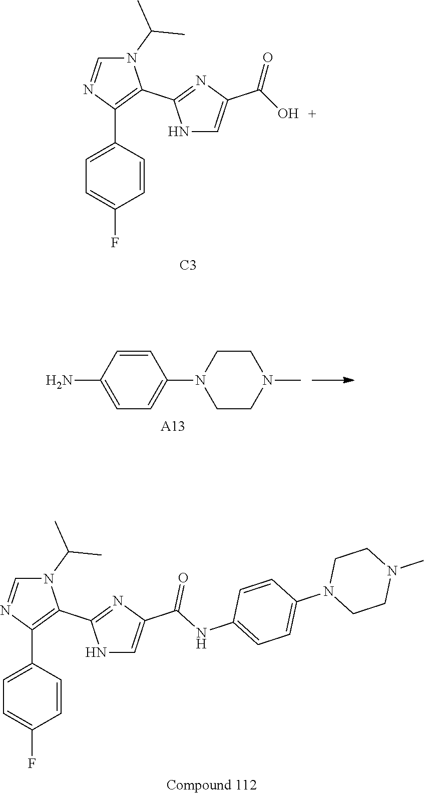

### Step 4-1: General Procedure for Preparation of ethyl 1-((2-(trimethylsilyl)ethoxy)methyl)-1H-imidazole-5-carboxylate (Compound A9)

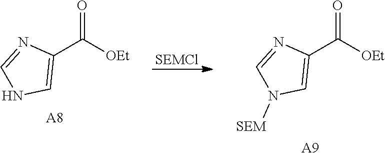

To a suspend of NaH (42.81 g, 1.07 mol, 60% purity, 1.5 eq) in THF (1 L) was added ethyl 1H-imidazole-5-carboxylate (compound A8) (100 g, 713.57 mmol, 1 eq) in portions at 0 °C, it was stirred at 20 T for 0.5 h, SEM-Cl (178.45 g, 1.07 mol, 189.44 mL, 1.5 eq) was added dropwise at 0 T, it was stirred at 25 °C for 16 h. TLC (PE:EA 1:1) showed compound A8 (Rf=0.1) was consumed completely and a new spot (Rf=0.3) was detected. It was poured into saturated NH₄Cl (1 L) aqueous, and extracted with ethyl acetate (1 L), the organic layer was washed with brine (1 L), dried over Na₂SO₄, filtered and concentrated in vacuum. The crude was purified by column chromatography (SiO₂, PE:EA 10:1-0:1). Compound A9 (136 g, 502.96 mmol, 70.48% yield) was obtained as a yellow solid, which was confirmed by ¹H-NMR. ¹H-NMR: (400 MHz, CHLOROFORM-d) 6=7.74 (d, J=1.3 Hz, 1H), 7.65 (d, J=1.1 Hz, 1H), 5.32 (s, 2H), 4.43-4.36 (m, 2H), 3.54-3.44 (m, 2H), 1.44-1.38 (m, 3H), 1.29-1.26 (m, 2H), 0.96-0.89 (m, 3H), 0.02~−0.02 (m, 9H).

In some cases, the ethyl ester group of Compound A8 could be replaced with methyl ester, n-PrOH ester, i-PrOH ester, t-BuOH ester, benzyl ester, benzylmethyl ester, 4-methylbenzylmethyl ester, etc.

The protection group SEM could be replaced by commercially available protection group, eg., methyloxycarbonyl, ethyloxycarbonyl, Cbz (benzyloxycarbonyl), Boc (tert-butoxycarbonyl), Fmoc (9-fluorenylmethyloxycarbonyl), Alloc (allyloxycarbonyl), Teoc (2-(trimethylsilyl)ethyloxycarbonyl), Troc (2,2,2-trichloroethoxycarbonyl), Tos (para-toluenesulfonyl), Tfa (2,2,2-trifluoroacetyl), Trt (trityl), Dmb (2,4-dimethocybenzyl), Pmb (p-Methoxybenzyl), Bn (benzyl).

### Step 4-2: General Procedure for Preparation of ethyl 2-bromo-1-((2-(trimethylsilyl)ethoxy)methyl)-1H-imidazole-4-carboxylate (Compound A10)

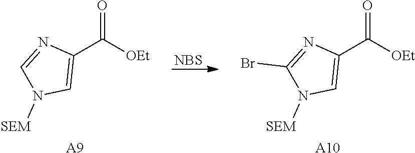

To a solution of compound A9 (116 g, 429.00 mmol, 1 eq) in CHCl₃ (1 L) was added NBS (83.99 g, 471.89 mmol, 1.1 eq) and AIBN (8.45 g, 51.48 mmol, 0.12 eq), it was stirred at 65 T for 4 h. TLC (PE:EA 3:1) showed compound A9 (Rf=0.1) was consumed and a new peak (Rf=0.4) was detected. The reaction mixture was quenched with brine (1 L), the organic layer was dried over Na₂SO₄, filtered and concentrated in vacuum. The crude was purified by column chromatography (SiO₂, PE:EA 20:1~2:1). Compound A10 (80 g, 229.03 mmol, 53.39% yield) was obtained as a yellow solid, which was confirmed by ¹H-NMR. ¹H-NMR: (400 MHz, DMSO-d₆) δ=8.30-8.24 (m, 1H), 8.20 (s, 1H), 5.38 (s, 2H), 4.30-4.22 (m, 2H), 3.63-3.53 (m, 2H), 1.33-1.28 (m, 3H), 0.89 (t, J=7.9 Hz, 2H), 0.02~−0.03 (m, 9H).

### Step 4-3: General Procedure for Preparation of ethyl 2-formyl-1-((2-(trimethylsilyl)ethoxy)methyl)-1H-imidazole-4-carboxylate (Compound B1)

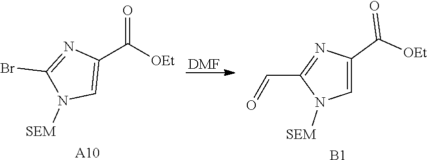

To a solution of Compound A10 (28 g, 80.16 mmol, 1 eq) in THF (300 mL) was added i-PrMgCl (2 M, 120.24 mL, 3 eq) at −40′C. The mixture was stirred at −40 °C for 10 min. To the mixture was added DMF (35.16 g, 480.97 mmol, 37.01 mL, 6 eq) at −70 °C. The mixture was stirred at 20 TC for 1 hrs. HPLC was showed Compound A10 was consumed. LCMS was showed desired mass was detected. The mixture was poured to 1N HCl (500 mL). The mixture was extracted with EA (300 mL×2). The combined organic layers were dried over Na₂SO₄, filtered and concentrated under reduced pressure to give a residue. The residue was purified by column chromatography on silica gel (PE:EA=10:1-3:1). Compound B1 (15 g, 50.27 mmol, 62.71% yield) was obtained as yellow oil, which was checked with LCMS and ¹H-NMR. LCMS: Retention time: 0.955 min, (M+H)=299.2. ¹H-NMR: (400 MHz, CHLOROFORM-d) δ=8.03-7.95 (m, 1H), 5.80 (s, 2H), 4.47-4.41 (m, 2H), 3.63-3.57 (m, 2H), 1.46-1.40 (m, 3H), 0.99-0.93 (m, 2H), 0.00 (s, 8H).

In some cases, the strong base i-PrMgCl for bromine-metal exchange reaction could be replaced with commercially available strong base, eg. LDA, n-BuLi, t-BuLi, LiHMDS, KHMDS, i-PrMgBr, MeMgCl, MeMgBr, EtMgCl, EtMgBr, etc.

DMF could be replaced with other formylation reagent, eg., N,N-diethylformamide, etc.

### Step 4-4: General Procedure for Preparation of ethyl 2-formyl-1H-imidazole-4-carboxylate (Compound C1)

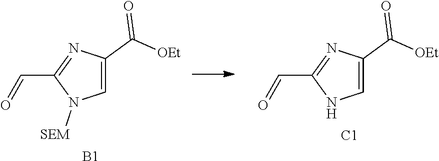

To a mixture of Compound B1 (40 g, 134.04 mmol, 1 eq) in 2-MeTHF (80 mL) was added 36.5% HCl aq. (90 mL, 9.1 eq). The mixture was stirred at 10 °C for 6-8 hours. Reaction mixture was adjusting pH to 7-8 with 15% NaOH aq. (333 mL) and saturated NaHCO₃ aq. (270 mL). Separate and extract the aqueous layers with EA (200 mL) fifth. Combine all organic phase and concentrate to dry below 40 °C and charge EtOH (40 mL). H₂O (400 mL) was charged into EtOH suspension of Compound C1 drop-wisely at 15-25 °C and stir at −5-5 °C for 16 h, and then filter wash with H₂O. Dry the wet cake at 45-55 °C for 20 hr. Assay yield is 85%. Structure was checked with LCMS. LCMS: (M+H)=169.2.

### Step 4-5: General Procedure for Preparation of ethyl 5′-(4-fluorophenyl)-3′-isopropyl-1H,3′H-[2,4′-biimidazole]-4-carboxylate (Compound C2)

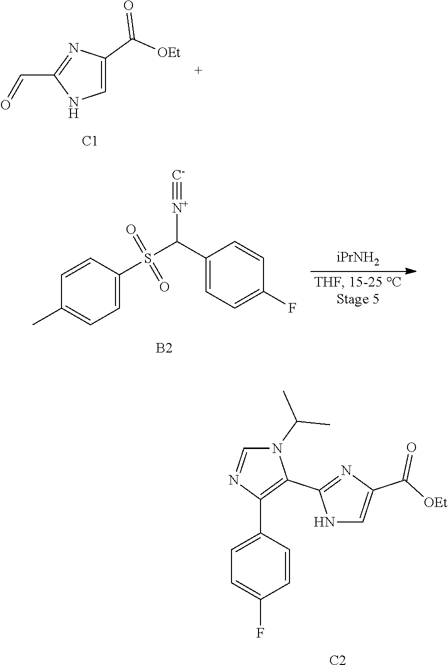

Compound C1 (100 g, 0.59 mol, 1 eq) and THF (1 L) were charged into R1, i-PrNH₂ (140.7 g, 2.38 mol, 4 eq.) was added and the mixture is stirred at 15-25 °C for 1 hour. Charge Compound B2 (172.1 g, 0.59 mol, 1.0 eq.) at 15-25 °C. The mixture was stirred at 15-25 °C for 2-11 hr. 500 mL MTBE and 1 L process water was added to the reaction mixture. Then adjust pH of the mixture to 1-2 with 35% aq. HCl and separated. The organic phase was washed by 500 mL 3.5% aq. HCl twice. Combine the aqueous phase and adjust pH to 7-8 with 30% aq. NaOH. Filter and dry the wet cake at 45-55 °C for 12-24 hr. Assay yield is 89%. Structures was checked with LCMS. LCMS: (M+H)=343.2.

In some cases, the 4-MePh group of Compound B2 could be replaced with other commercially available or easily prepared substituted phenyl group, the substitution could be H, F, Cl, Br, methyl, methoxy, etc.

The reaction solvent could be selected from commercially available solvent, eg. THF, 2-MeTHF, toluene, 1,4-dioxane, 1,2-DCE, DCM, DMF, DMAc, NMP, DMSO, acetone, acetonitrile, EtOH, MeOH, i-PrOH, t-BuOH, etc or their combination.

Organic base, eg. TEA, DIEA etc. could also be added to accelerate the reaction.

The reaction temperature can be −20 °C ~110 °C.

The crystallization solvents can be selected from the following solvent: EA, IPAc, MTBE, THF, Me-THF, ethyl ether, n-heptane, n-hexane, Methylcyclohexane, Petroleum ether, MeOH, EtOH, i-PrOH, acetone, acetonitrile, etc., and their combination.

### Step 4-6: General Procedure for Preparation of 5′-(4-fluorophenyl)-3′-isopropyl-1H,3′H-[2,4′-biimidazole]-4-carboxylic acid (Compound C3)

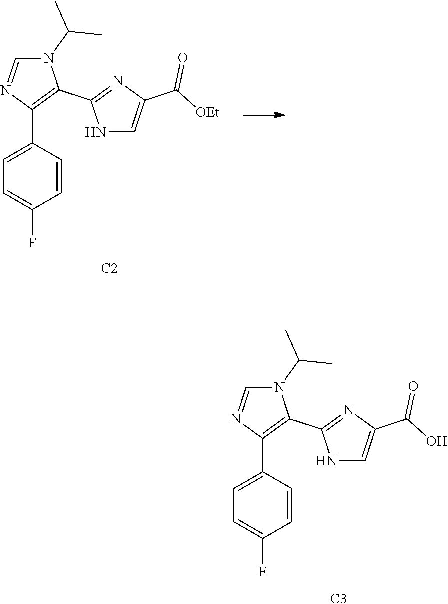 [SMILES: CCOC(=O)c1c[nH]c(-c2c(-c3ccc(F)cc3)ncn2C(C)C)n1 ; "C2" ; CC(C)n1cnc(-c2ccc(F)cc2)c1-c1nc(C(=O)O)c[nH]1 ; "C3"]

Compound C2 (100 g, 0.29 mol, 1 eq) and 1,4-dioxane (500 mL) were charged into Reactor, 13.48% NaOH aq. (346.7 g, 1.17 mol, 4 eq) was added and the mixture is stirred at 75-85 T for 16 hours. Cool to 20-50 °C and 700 mL process water was added to the reaction mixture. Then charge 36-39% HCl aq. (115 g, 1.17 mol, 4.0 eq) at 20-50 °C. Adjust Reactor to 0-10 °C and stir for 2-4 hrs at 0-10 °C. Then filter and wash wet cake with 150 mL process water. Dry the wet cake at 50-55 TC for 24 hr. Assay yield is 90%. Structures was checked with LCMS. LCMS: (M+H)=315.2.

The base NaOH for hydrolysis reaction can be replaced with KOH, LiOH, Ca(OH)₂, K₂CO₃, KHCO₃, Na₂CO₃, NaHCO₃, K₃PO₄, K₂HPO₄, KH₂PO₄, Na₃PO₄, Na₂HPO₄, NaH₂PO₄, etc.

The reaction solvent can be chosen from 1,4-dioxane, MeOH, EtOH, i-PrOH, n-PrOH, THF, water and their combination.

The reaction temperature can be −20 ~110 °C.

### Step 4-7: General Procedure for Preparation of 5′-(4-fluorophenyl)-3′-isopropyl-N-(4-(4-methylpiperazin-1-yl)phenyl)-1H,3′H-[2,4′-biimidazole]-4-carboxamide (Compound 112)

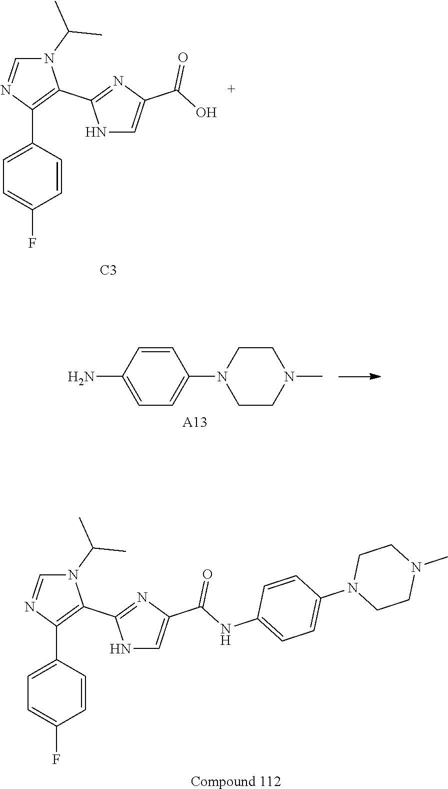

To suspension of Compound C3 (79 g, 76.4% assay, 0.192 mol, 1.0 eq) in THF (790 mL) was added Et₃N (77.6 g, 0.77 mol, 4.0 eq.). The mixture was stirred at 15-25 °C for 30-60 min and then cooled to −5-5 °C. Compound A13 (47.7 g, 0.25 mol, 1.3 eq) was added and then 50% T₃P EA solution (183 g, 0.29 mol, 1.5 eq.) was added drop-wise at −5-5 °C. The mixture was stirred at −5-5 °C until the reaction mixture converted completely. EA (560 mL) and 560 mL 5% Na₂SO₄ aqueous solution was charged and then stirred and separated. The aqueous phase was extracted with 240 mL EA and 400 mL THF. The organic phase was combined and then washed with 7% NaHCO₃ (560 mL) and water (560 mL). Resulting organic phase decolored through CUNO (12 g active cake) and then switch into EtOH (560 mL) and then water (800 mL) was added drop-wise at 15-25 °C. The mixture was stirred at 15-25 °C for 2-4 hrs, and then filtered and washed with water (400 mL). Dissolved the wet cake with 400 mL THF at 45-55 °C and then water (800 mL) was added drop-wise at 20-30 °C. The mixture was stirred at 20-30 °C for 4-10 hrs, and then filtered and washed with water (400 mL). The cake was dried at 45-60 °C for 16-24 hrs under vacuum to afford 36.9 g target Compound 112 with 99.85% purity and 78.8% assay yield. Structure was checked with LCMS and H-NMR. LCMS: Retention time: 0.683 min, (M+H)=488.2. ¹H-NMR: (400 MHz, DMSO-d₆) δ=13.14-12.98 (m, 1H), 9.72 (s, 1H), 8.07 (s, 1H), 7.98 (s, 1H), 7.67 (d, J=9.2 Hz, 2H), 7.44-7.34 (m, 2H), 7.14 (t, J=8.8 Hz, 2H), 6.90 (d, J=9.0 Hz, 2H), 4.30-4.18 (m, 1H), 3.13-3.04 (m, 4H), 2.47-2.42 (m, 4H), 2.22 (s, 3H), 1.40 (d, J=6.4 Hz, 6H).

The condensation reagent T₃P could be replaced by commercially available condensation reagent, eg. HATU, EDCI, PyBOP, HBTU, DPPA, CDI, etc.

T₃P/EA solution could be replaced with other T₃P solution, eg. T₃P, T₃P/THF solution, T₃P/2-MeTHF solution, T₃P/IPAc solution.

### Example 5: New Synthesis Route 4 for Compound 112

Isonitrile chemistry reaction between Compound D1 with Compound B2 and i-PrNH₂ gave Compound D2. Hydrolysis of the CF₃ group to carboxylic acid group and then condensation with Compound A13 gave target compound.

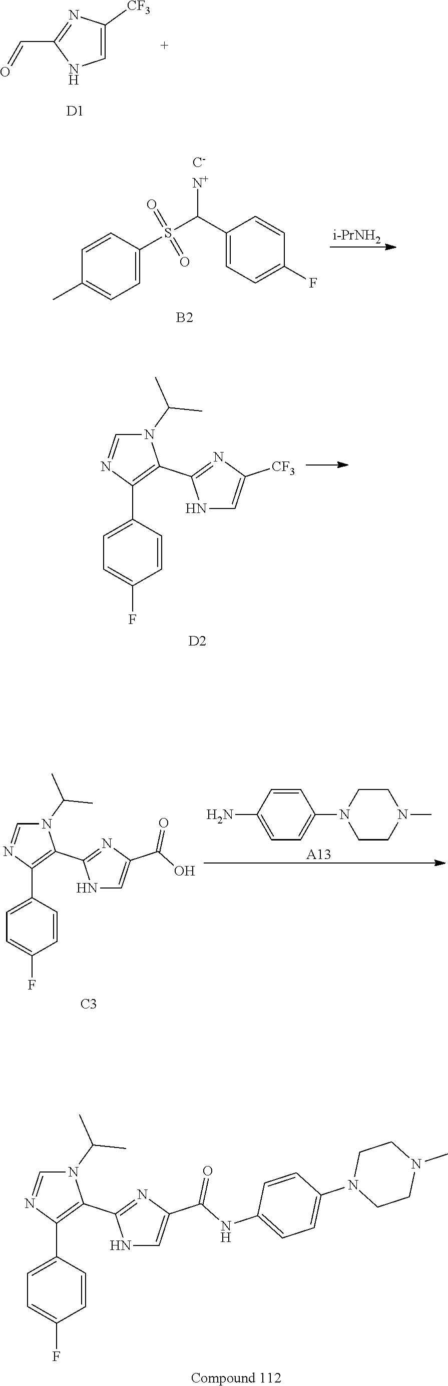

### Step 5-1: General Procedure for Preparation of 5′-(4-fluorophenyl)-3′-isopropyl-4-(trifluoromethyl)-1H,3′H-2,4′-biimidazole (Compound D2)

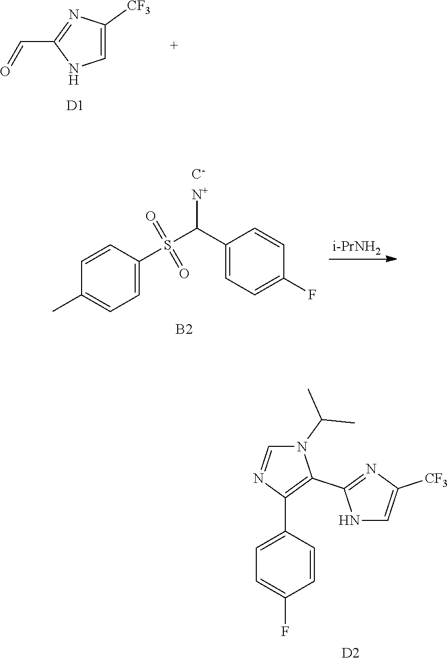

Compound D1 was prepared according to procedure reported in WO2009084614A1.

To a mixture of Compound D1 (10.0 g, 60.94 mmol, 1 eq) in THF (70.0 mL) was added i-PrNH₂ (14.41 g, 243.78 mmol, 4.0 eq). The mixture was stirred at 20 TC for 1-2 hrs. To the mixture was added Compound B2 (18.51 g, 67.05 mmol, 1.05 eq). The mixture was stirred at 20 TC for 2-6 h until the reaction mixture converted completely. EA (50 mL) and water (50 mL) was charged. The pH of mixture was adjusted to 5-6 with 36% HCl aq. (11.5 g) and then separated. The organic phase was adjusted pH to 9-10 with 10% aq. Na₂CO₃ (40 g) aq. and then separated. The organic phase was washed with 5% aq. Na₂CO₃ (20 g) and then separated. The organic phase was switched into EA solution (30 mL) and then n-heptane (30 mL) was charged. The suspension was concentrated to ~30 mL below 45 °C under vacuum and then n-heptane (50 mL) was added drop-wise at 15-25 °C. The mixture was cooled to 0-10 °C with 2.0 hr and stirred at 0-10 °C for 4-6 hrs, then filtered and washed with n-heptane (20 mL). The wet cake was dried at 40-50 °C for 10-18 hrs under vacuum. Assay yield is 91%. Structure was checked with LCMS. LCMS: (M+H)=339.2.

As a further example, the reaction condition could be adjusted as below: The CF₃ group of Compound D1 could be replaced by CR^{A1}R^{A2}R^{A3}, and R^{A1}, R^{A2} and R^{A3} could be the same or different group, selecting from F, Cl, Br, OMe, OEt, e.g., CBr₃, CCl₃, CF₂Cl, CFCl₂, CF₂Br, CFBr₂, C(OMe)₃, C(OEt)₃, etc.

4-MePh group of Compound B2 could be replaced with other commercially available or easily prepared substituted phenyl group, the substitution could be H, F, Cl, Br, methyl, methyloxy, etc.

The reaction solvent can be selected from commercially available solvent, eg. THF, 2-MeTHF, toluene, 1,4-dioxane, 1,2-DCE, DCM, DMF, DMAc, NMP, DMSO, acetone, acetonitrile, EtOH, MeOH, i-PrOH, t-BuOH, etc and their combination.

Organic base, eg. TEA, DIEA etc. can also be added to accelerate the reaction.

The reaction temperature can be −20 °C-110 °C.

The crystallization solvents can be of the following solvent: EA, IPAc, MTBE, THF, Me-THF, ethyl ether, n-heptane, n-hexane, methylcyclohexane, petroleum ether, MeOH, EtOH, i-PrOH, acetone, acetonitrile, etc. and their combination.

### Step 5-2: General Procedure for Preparation of 5′-(4-fluorophenyl)-3′-isopropyl-1H,3′H-[2,4′-biimidazole]-4-carboxylic acid (Compound C3)

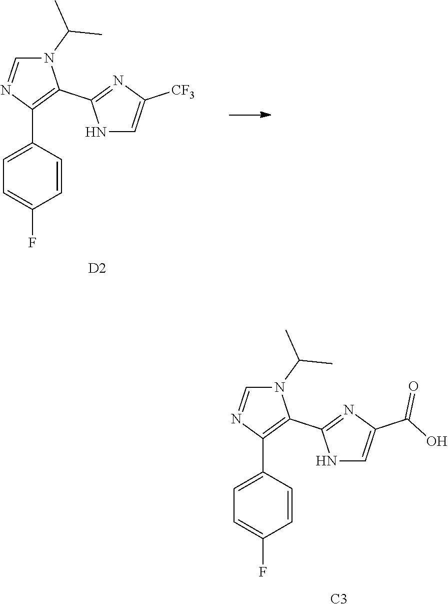 [SMILES: CC(C)n1cnc(-c2ccc(F)cc2)c1-c1nc(C(F)(F)F)c[nH]1 ; "D2" ; CC(C)n1cnc(-c2ccc(F)cc2)c1-c1nc(C(=O)O)c[nH]1 ; "C3"]

To a mixture of Compound D2 (10.0 g, 29.56 mmol, 1 eq) in MeOH (50 mL) was added the solution of NaOH (7.09 g, 177.36 mmol, 6.0 eq) dissolved in water (50 mL) and the mixture was stirred at 60-70 TC for 16 hrs until Compound D2 was consumed completely. The mixture and adjusted to pH 4.7-5.5 by addition aq. HCl and filtered. The cake was washed with water (20 g) and then slurred with water (100 g) for 6-8 hrs at 35-45 °C and stir for 4-6 hrs at 15-25 °C. The wet cake was filter and dried at 40-50 °C for 12-18 hrs under vacuum. Assay yield is 90%. Structure was checked with LCMS.

LCMS: (M+H)=315.3.

In some cases, the base NaOH for hydrolysis reaction could be replaced with KOH, LiOH, Ca(OH)₂, K₂CO₃, KHCO₃, Na₂CO₃, NaHCO₃, K₃PO₄, K₂HPO₄, KH₂PO₄, Na₃PO₄, Na₂HPO₄, NaH₂PO₄, etc.

The reaction solvent can be chosen from 1,4-dioxane, MeOH, EtOH, i-PrOH, n-PrOH, THF, water and their combination.

The reaction temperature can be −20~110 °C.

### Step 5-3: General Procedure for Preparation of 5′-(4-fluorophenyl)-3′-isopropyl-N-(4-(4-methylpiperazin-1-yl)phenyl)-1H,3′H-[2,4′-biimidazole]-4-carboxamide (Compound 112)

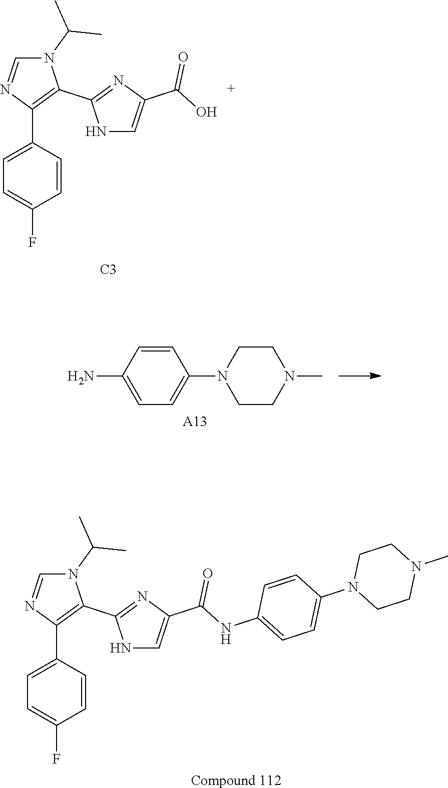

To suspension of Compound C3 (79 g, 1.0 eq, 76.4% assay, 0.192 mol) in THF (790 mL) was added Et₃N (77.6 g, 0.77 mol, 4.0 eq.). The mixture was stirred at 15-25 °C for 30-60 mins and then cooled to −5-5 °C. Compound A13 (47.7 g, 0.25 mol, 1.3 eq) was added and then 50% T3P EA solution (183 g, 0.29 mol, 1.5 eq.) was added drop-wise at −5-5 °C. The mixture was stirred at −5-5 °C until the reaction mixture converted completely. EA (560 mL) and 560 mL 5% Na₂SO₄ aqueous solution was charged and then stirred and separated. The aqueous phase was extracted with 240 mL EA and 400 mL THF. The organic phase was combined and then washed with 7% NaHCO₃ (560 mL) and water (560 mL). Resulting organic phase decolored through CUNO (12 g active cake) and then switch into EtOH (560 mL) and then water (800 mL) was added drop-wise at 15-25 °C. The mixture was stirred at 15-25 °C for 2-4 hrs, and then filtered and washed with water (400 mL). Dissolve the wet cake with 400 mL THF at 45-55 °C and then water (800 mL) was added drop-wise at 20-30 °C. The mixture was stirred at 20-30 °C for 4-10 hrs, and then filtered and washed with water (400 mL). The cake was dried at 45-60 °C for 16-24 hrs under vacuum to afford 36.9 g target Compound 112 with 99.85% purity and 78.8% assay yield. Structure was checked with LCMS and ¹H-NMR. LCMS: Retention time: 0.683 min, (M+H)=488.2. ¹H-NMR: (400 MHz, DMSO-d₆) δ=13.14-12.98 (m, 1H), 9.72 (s, 1H), 8.07 (s, 1H), 7.98 (s, 1H), 7.67 (d, J=9.2 Hz, 2H), 7.44-7.34 (m, 2H), 7.14 (t, J=8.8 Hz, 2H), 6.90 (d, J=9.0 Hz, 2H), 4.30-4.18 (m, 1H), 3.13-3.04 (m, 4H), 2.47-2.42 (m, 4H), 2.22 (s, 3H), 1.40 (d, J=6.4 Hz, 6H).

In some cases, the condensation reagent T₃P could be replaced by commercially available condensation reagent, eg. HATU, EDCI, PyBOP, HBTU, DPPA, CDI, etc.

T₃P/EA solution can be replaced with other T₃P solution, eg. T₃P, T₃P/THF solution, T₃P/2-MeTHF solution, T₃P/IPAc solution.

### Example 6: New Route 4 of Compound 112 could Also be Used in the Synthesis of Compound 120

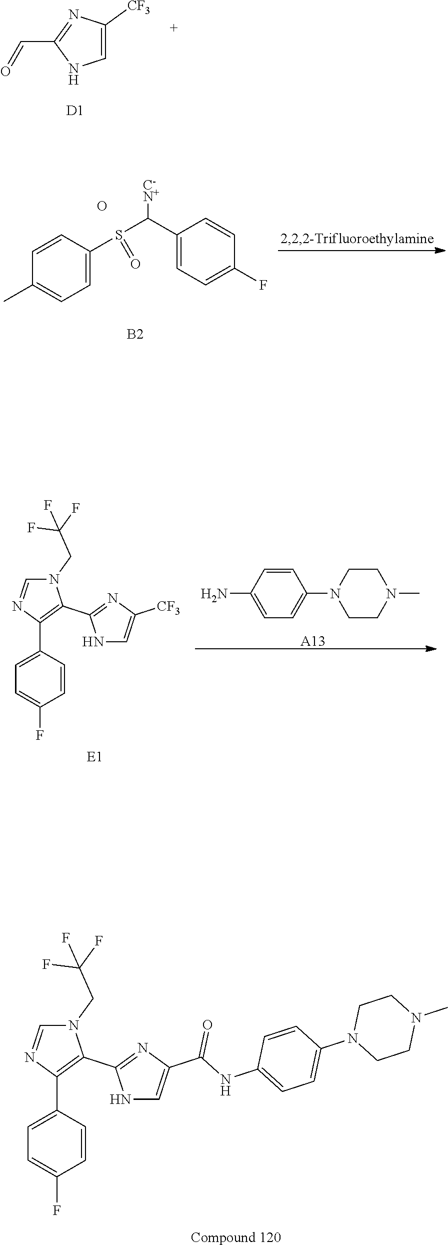

Isonitrile chemistry reaction between Compound D1 with Compound B2 and 2,2,2-trifluoroethylamine gave Compound E1. Hydrolysis of the CF₃ group to carboxylic acid group and then condensation with Compound A13 gave target compound Compound 120.

### Step 6-1: General Procedure for Preparation of 5′-(4-fluorophenyl)-3′-neopentyl-4-(trifluoromethyl)-1H,3′H-2,4′-biimidazole (Compound E1)

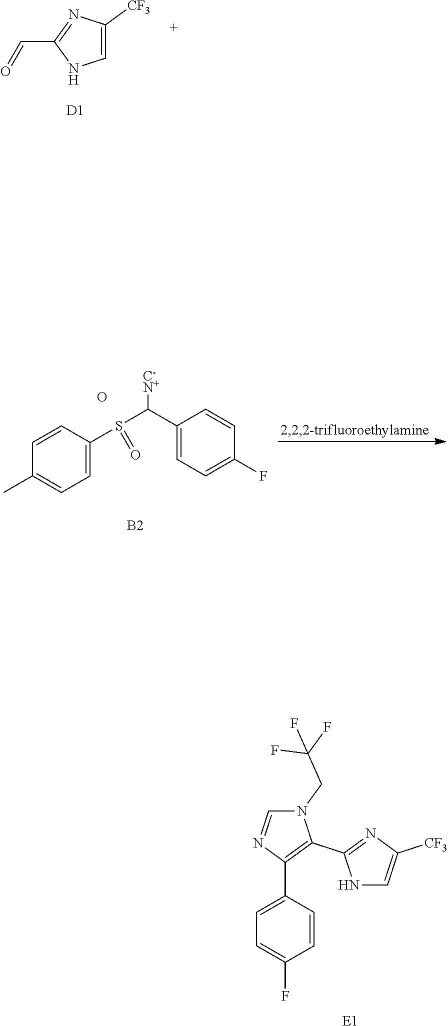

Compound D1 was prepared according to procedure reported in WO2009084614A1.

To a solution of Compound D1 (100 g, 0.61 mol, 1.0 eq.) in THF (1 L), 2,2,2-trifluoroethanamine (362 g, 3.7 mol, 6.0 eq) was added. The mixture was stirred at 15-25 °C for 3-4 hrs, and then DIEA (315 g, 2.4 mol, 4.0 eq.) and Compound B2 (194 g, 0.67 mol, 1.1 eq) was added. The reaction was stirred at 30-35 °C for 40-50 hrs, and then 2,2,2-trifluoroethanamine (122 g, 1.22 mol, 2.0 eq) was added. Resulting mixture was stirred at 30-35 °C until HPLC judged reaction was complete. Then MTBE (500 g) and water (700 g) was added at 20-25 °C. The pH of reaction mixture was adjusted to 5-7 with 35% aq. HCl (170 g) and then stirred and separated. Resulting aqueous phase was extracted with MTBE (500 g) again. Obtained organic layer was combined and washed with water (500 g) and then concentrated and switch to toluene (100 mL) by distillation. Resulting mixture was heated and stirred at 40-45 °C for 1 hr and then cooled slowly to 10-15 °C. MCH (365 g) was added slowly followed by slowly cooling to 0-5 °C. MCH (365 g) was added slowly at 0-5 °C and then stirred for 6-14 hrs. The mixture was filtered. The cake was washed with MCH (150 mL). The cake was dried under vacuum at 45-55 °C to give solid A. Assay yield is 85%. Structure was checked with LCMS. LCMS: (M+H)=367.2.

### Step 6-2: General Procedure for Preparation of 5′-(4-fluorophenyl)-3′-(2,2,2-trifluoroethyl)-1H,3′H-[2,4′-biimidazole]-4-carboxylic acid (Compound E2)

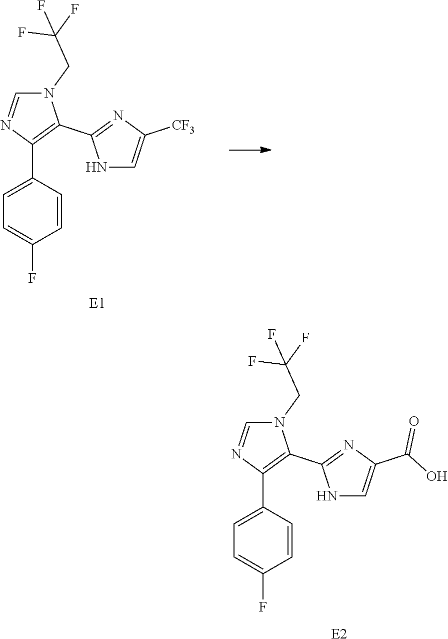 [SMILES: Fc1ccc(-c2ncn(CC(F)(F)F)c2-c2nc(C(F)(F)F)c[nH]2)cc1 ; "E1" ; O=C(O)c1c[nH]c(-c2c(-c3ccc(F)cc3)ncn2CC(F)(F)F)n1 ; "E2"]

To a mixture of Compound E1 (100 g, 0.27 mol, 1.0 eq.) in MeOH (600 mL) was added the solution of 9.56% aq. NaOH (697 g, 1.67 mol, 6.1 eq). The mixture was heated and stirred at 50-55 T until HPLC judged reaction converted completely. Water (500 mL) was added and the mixture was adjusted to pH 4-5 using 35% aq. HCL. The mixture was stirred at 15-25 °C for 2-4 hrs and filtered. The filter cake was washed with water (200 mL) twice and then dried at 40-50 °C for 12-18 hrs under vacuum to give solid Compound E2. Assay yield is 90%. Structure was checked with LCMS. LCMS: (M+H)=355.2.

### Step 6-3: General Procedure for Preparation of 5′-(4-fluorophenyl)-N-(4-(4-methylpiperazin-1-yl)phenyl)-3′-(2,2,2-trifluoroethyl)-1H,3′H-[2,4′-biimidazole]-4-carboxamide (Compound 120)

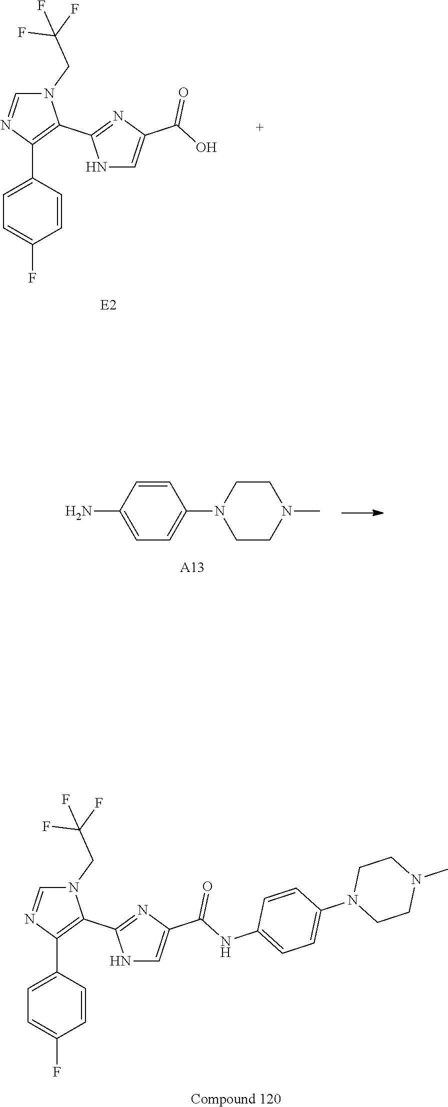

Distill suspension of Compound E2 (100 g, 0.28 mol, 1.0 eq.) with THF (850 mL) twice to reduce water content. To suspension of Compound E2 in THF (1 L) was added Et₃N (114 g, 1.13 mol, 4.0 eq.) and Compound A13 (80.9 g, 0.42 mol, 1.5 eq). The mixture was stirred at 15-25 °C for 30-60 mins and then cooled to −5-5 °C. Compound A13 (72.8 g, 0.38 mol, 1.35 eq) was added and then 50% T₃P EA solution (121 g, 0.38 mol, 1.35 eq.) was added drop-wise at −5-5 °C. The mixture was stirred at −5-5 °C until the reaction mixture converted completely. EA (630 mL), 5% Na₂SO₄ aqueous solution (354 g) and water (320 g) were charged and then stirred and separated. The aqueous phase was extracted with EA (250 mL) and THF (450 mL). The organic phase was combined and then washed with 7% NaHCO₃ (700 g*2) and water (475 g*2). The washed aqueous phase was combined and extracted with EA (70 g*2). The organic phase was combined and decolored through CUNO (15 g active cake). THF (450 g) was washed the CUNO (0.15 g active cake), and combined the Compound 120 EA/THF solution. The Compound 120 EA/THF solution was concentrated and switched into EtOH (700 mL) and then water (1050 mL) was added drop-wise at 15-25 °C. The mixture was stirred at 15-25 °C for 2-4 hrs, and then filtered and washed with water (200 g). The cake was dried at 30-60 °C for 10-18 hrs under vacuum and 20-50% RH. Assay yield is 75%. Structure was checked with LCMS. LCMS: (M+H)=528.2.

As illustrated above, procedures of Examples 2-6 can provide short synthesis routes, relatively higher yield, and easy to scale up. Further, the Sn reagent required for Stille coupling in order to form a biimidazole core is not needed in procedures of Examples 2-6.

In addition, it is easy to perform quality control for procedures of Examples 2-6.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of producing a compound represented by Formula (I), or a pharmaceutically acceptable salt thereof, 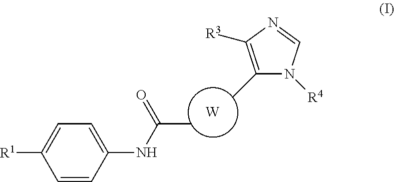
wherein
R¹ is selected from:
N(R⁵)₂, wherein each R⁵ is independently selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle; and
optionally substituted 3 to 8-membered heterocycle; wherein the 3 to 8-membered heterocycle is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, and —NO₂;
R³ is selected from optionally substituted C₁-C₆ alkyl, optionally substituted 3- to 10-membered heterocycle and optionally substituted C₃₋₁₀ carbocycle, wherein each of the alkyl, heterocycle and carbocycle is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R⁴ is selected from:
hydrogen;
optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀alkynyl, C₃₋₁₂ carbocycle, and 3- to 12-membered heterocycle;
Ring W is an optionally substituted 5 or 6-membered heteroaryl, wherein the 5 or 6-membered heteroaryl is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂-10 alkenyl, C₂-10 alkynyl, C₃₋₁₂ carbocycle, and 3- to 12-membered heterocycle,
wherein the method comprises subjecting a compound represented by Formula (A), or a pharmaceutically acceptable salt thereof, 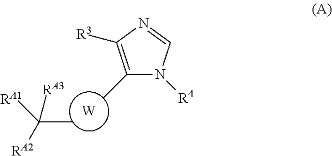
wherein Ring W, R³ and R⁴ are defined above, and
each of R^{A1}, R^{A2} and R^{A3} is independently selected from a halogen and —O—C₁₋₆alkyl;
through a hydrolysis step to produce a compound represented by Formula (B), or a pharmaceutically acceptable salt thereof, 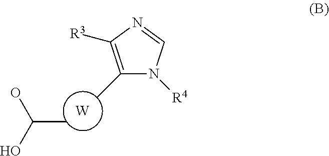
wherein Ring W, R³ and R⁴ are defined above.

2. The method of claim 1, each of R^{A}, R^{A2} and R^{A3} is independently selected from halogen and —O—C₁₋₃alkyl.

3. The method of claim 1, each of R^{A}, R^{A2} and R^{A3} is independently selected from a halogen.

4. The method of claim 1, each of R^{A}, R^{A2} and R^{A3} is independently selected from a —O—C₁₋₆alkyl.

5. The method of claim 1, each of R^{A}, R^{A2} and R^{A3} is independently selected from F, Cl, Br, OMe, and OEt.

6. The method of claim 1, 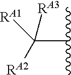
is CBr₃, CCl₃, CF₃, CF₂C₁, CFCl₂, CF₂Br, CFBr₂, C(OMe)₃, or C(OEt)₃.

7. The method of claim 1, 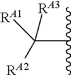
is CF₃.

8. The method of any one of claims 1 to 7, wherein the hydrolysis step is performed in a solvent selected from ether, alcohol, water, and a mixture thereof. (e.g., MeOH/Water).

9. The method of claim 8, wherein the hydrolysis step is performed in a solvent selected from 1,4-dioxane, MeOH, EtOH, i-PrOH, n-PrOH, THF, water, or a mixture thereof.

10. The method of claim 8, wherein the hydrolysis step is performed in a mixture of methanol and water.

11. The method of any one of claims 1 to 10, wherein the hydrolysis step is performed in an alkaline condition.

12. The method of any one of claims 1 to 11, wherein the hydrolysis step is performed in the presence of a base, wherein the base is an alkali, an alkaline salt, or a mixture thereof.

13. The method of claim 12, wherein the alkaline salt is a carbonate salt, bicarbonate salt, or phosphate salt (e.g., phosphate, hydrogenphosphate and dihydrogenphosphate).

14. The method of claim 12, wherein the base is selected from NaOH, KOH, LiOH, Ca(OH)₂, K₂CO₃, KHCO₃, Na₂CO₃, NaHCO₃, K₃PO₄, K₂HPO₄, KH₂PO₄, Na₃PO₄, Na₂HPO₄, NaH₂PO₄, or a combination thereof.

15. The method of claim 12, wherein the base is NaOH.

16. The method of any one of claims 1 to 15, further comprising subjecting the compound represented by Formula (B), or a pharmaceutically acceptable salt thereof, through a condensation reaction with a compound of Formula (C), or a pharmaceutically acceptable salt thereof, 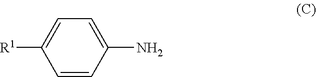
wherein R¹ is defined above in Formula (I).

17. The method of claim 16, wherein the condensation step is performed in the presence of a base (e.g., DIEA), a coupling agent (HATU), or both.

18. The method of claim 16, wherein the condensation step is performed in the presence of a condensation reagent (e.g., T₃P, HATU, EDCI, PyBOP, HBTU, DPPA, CDI).

19. The method of any one of claims 1 to 18, wherein the compound represented by Formula (I) has a structure of Formula (Ia), 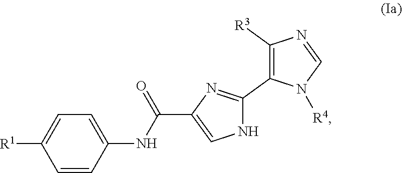
wherein:
R¹ is piperazine, wherein the piperazine is optionally substituted with one or more substituents independently selected at each occurrence from oxo and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is substituted C₁-C₆ alkyl, wherein the C₁-C₆ alkyl is substituted with one or more halogen.

20. The method of any one of claims 1 to 18, wherein the compound represented by Formula (I) has a structure of Formula (Ia), 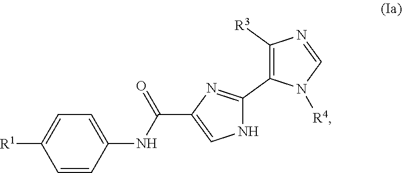
wherein,
R¹ is piperazine, wherein the piperazine is optionally substituted with one or more substituents independently selected at each occurrence from oxo, —S(O₂)NH₂, and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is unsubstituted C₁-C₆ alkyl.

21. A method of producing a compound represented by Formula (I), or a pharmaceutically acceptable salt thereof, 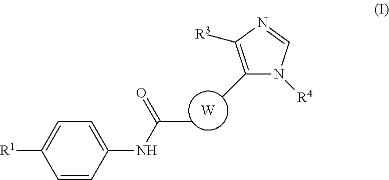
wherein:
R¹ is selected from:
N(R⁵)₂, wherein each R⁵ is independently selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle; and
optionally substituted 3 to 8-membered heterocycle; wherein the 3 to 8-membered heterocycle is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂-10 alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀haloalkyl, —NH₂, —CN, and —NO₂;
R³ is selected from optionally substituted C₁-C₆ alkyl, optionally substituted 3- to 10-membered heterocycle and optionally substituted C₃₋₁₀ carbocycle, wherein each of the alkyl, heterocycle and carbocycle is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R⁴ is selected from:
hydrogen;
optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₁₋₀ alkynyl, C₃₋₁₂ carbocycle, and 3- to 12-membered heterocycle; and
Ring W is an optionally substituted 5 or 6-membered heteroaryl, wherein the 5 or 6-membered heteroaryl is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle,
wherein the method comprises reacting a compound represented by Formula (E), or a pharmaceutically acceptable salt thereof, 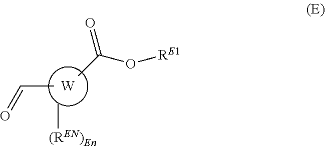
wherein,
Ring W is defined above,
En is 0 or 1,
R^{EN} is a protecting group, and
R^{E1} is C₁-C₆ alkyl (e.g., ethyl), C₁-C₆haloalkyl, C₁-C₆hydroxyalkyl, C₁-C₆aminoalkyl, C₁-C₆heteroalkyl, substituted or unsubstituted aryl, or substituted or unsubstituted heteroaryl;
with a compound represented by Formula (F), or a pharmaceutically acceptable salt thereof, 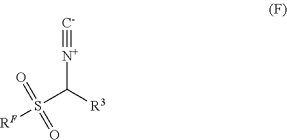
wherein R³ is defined above, and
R^{F} is a substituted or unsubstituted phenyl,
in the presence of a compound of Formula (K), or a pharmaceutically acceptable salt thereof,
R⁴—NH₂ (K),
thereby producing a compound represented by Formula (G), or a pharmaceutically acceptable salt thereof, 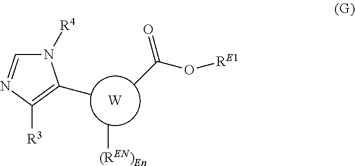
wherein R³, R⁴, Ring W, En, R^{EN} and R^{E1} are defined above.

22. The method of claim 21, wherein R^{E1} is methyl, ethyl, n-propyl, i-propyl, n-butyl, sec-butyl, i-butyl, or t-butyl.

23. The method of claim 21, wherein R^{E1} is ethyl.

24. The method of claim 21, wherein R^{E1} is phenyl, benzyl, or p-methoxybenzyl.

25. The method of any one of claims 21 to 24, wherein En is 0.

26. The method of any one of claims 21 to 24, wherein En is 1.

27. The method of any one of claims 21 to 24, wherein the compound of Formula (E) has a structure of Formula (E-1), 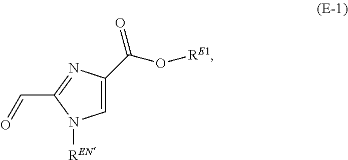
wherein R^{EN′} is hydrogen or R^{EN}.

28. The method of claim 27, wherein the compound of Formula (E-1) has a structure

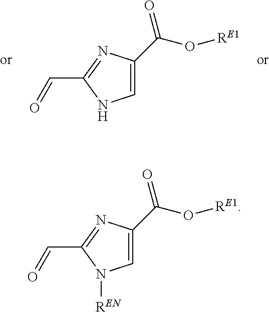

29. The method of any one of claims 21 to 23 or 26 to 28, wherein R^{EN} is selected from (trimethyl silicon)ethoxymethyl (SEM), methyloxycarbonyl, ethyloxycarbonyl, benzyloxycarbonyl (CBz), tert-butoxycarbonyl (Boc), 9-fluorenylmethyloxycarbonyl (Fmoc), allyloxycarbonyl (Alloc), 2-(trimethylsilyl)ethyloxycarbonyl (Teoc), 2,2,2-trichloroethoxycarbonyl (Troc), para-toluenesulfonyl (Tos), 2,2,2-trifluoroacetyl (Tfa), trityl (Trt), 2,4-dimethocybenzyl (Dmb), p-Methoxybenzyl (Pmb), and benzyl (Bn).

30. The method of claim 29, wherein R^{EN} is SEM.

31. The method of any one of claims 21 to 30, wherein R^{F} is optionally substituted with one or more R^{F1}, and each R^{F1} is independent selected from halogen, —NH₂, CN, NO₂, —OH, —SH, SF₅, C₁-C₆alkyl, —OC₁-C₆alkyl, C₁-C₆haloalkyl, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆hydroxyalkyl, C₁-C₆aminoalkyl, and C₁-C₆heteroalkyl.

32. The method of claim 31, wherein R^{F} is optionally substituted with one or more R^{F}a, and each R^{F1} is independent selected from F, Cl, Br, methyl, and methyloxy.

33. The method of claim 31 or 32, wherein the compound of Formula (F) has a structure of Formula 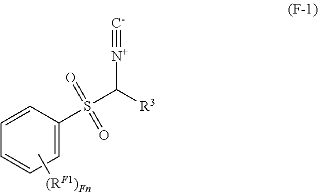
wherein Fn is 0, 1, 2, 3, 4, or 5.

34. The method of claim 31, wherein the compound of Formula (F) is

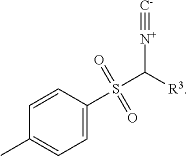

35. The method of any one of claims 21 to 34, wherein the compound represented by Formula (G) has a structure of Formula (G-1) 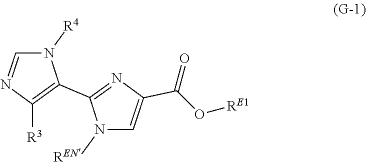
wherein R^{EN′} is hydrogen or R^{EN}.

36. The method of claim 35, wherein the compound represented by Formula (G-1) has a structure of

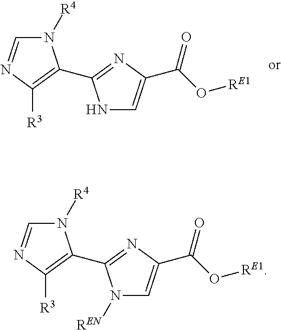

37. The method of any one of claims 21 to 36, further comprising subjecting the compound represented by Formula (G) or a pharmaceutically acceptable salt thereof, to a hydrolysis rection, thereby producing a compound of Formula (H), or a pharmaceutically acceptable salt thereof, 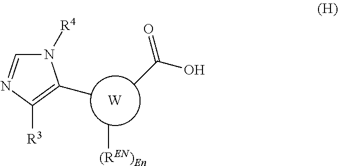
wherein R³, R⁴, Ring W, En, and R^{EN} are defined above.

38. The method of claim 37, wherein the compound represented by Formula (H) has a structure of Formula (H-1),

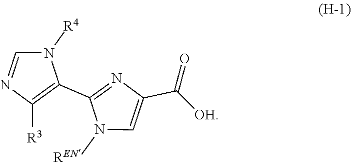

39. The method of claim 38, wherein the compound represented by Formula (H-1) has a structure of

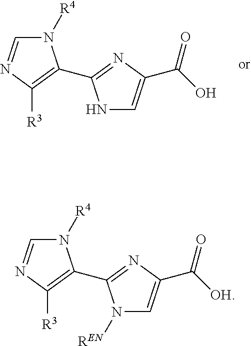

40. The method of any one of claims 21 to 39, further comprising subjecting the compound represented by Formula (H) or a pharmaceutically acceptable salt thereof, through a condensation reaction with a compound of Formula (C), or a pharmaceutically acceptable salt thereof, 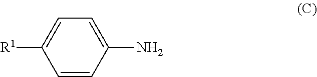
wherein R¹ is defined above in Formula (Ia).

41. The method of any one of claims 21 to 23 or 26 to 40, wherein the method further comprises a deprotecting reaction.

42. The method of claim 41, wherein the deprotecting reaction comprises replacing R^{EN} with a hydrogen.

43. The method of claim 41 or 42, wherein the deprotecting reaction occurs after the condensation reaction.

44. The method of any one of claims 21 to 43, wherein the compound represented by Formula (I) has a structure of Formula (Ia), 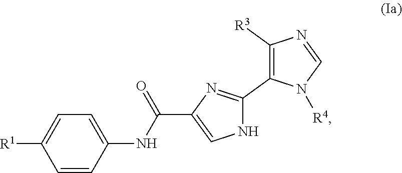
wherein:
R¹ is piperazine, wherein the piperazine is optionally substituted with one or more substituents independently selected at each occurrence from oxo and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is substituted C₁-C₆ alkyl, wherein the C₁-C₆ alkyl is substituted with one or more halogen.

45. The method of any one of claims 21 to 43, wherein the compound represented by Formula (I) has a structure of Formula (Ia), 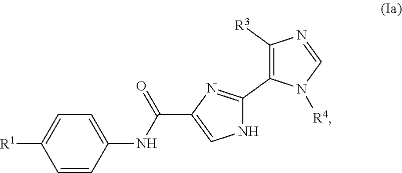
wherein,
R¹ is piperazine, wherein the piperazine is optionally substituted with one or more substituents independently selected at each occurrence from oxo, —S(O₂)NH₂, and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is unsubstituted C₁-C₆ alkyl.

46. A method of producing a compound represented by Formula (I), or a pharmaceutically acceptable salt thereof, 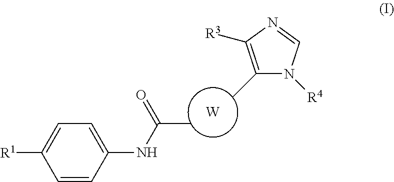
wherein:
R¹ is selected from:
N(R⁵)₂, wherein each R⁵ is independently selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₄₀ alkenyl, C₂₋₁₀ alkynyl, C₃₋₁₂ carbocycle, and 3- to 12-membered heterocycle;
substituted C₁-C₆ alkyl, wherein the C₁-C₆ alkyl is optionally substituted with one or more substituents independently selected at each occurrence from halogen, —OH, —CN, —NO₂, —NH₂, oxo, ═S, -C₁₋₁₀ haloalkyl, —O—C₁₋₁₀ alkyl, —O—C₁₋₁₀ alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle; and
optionally substituted 3 to 8-membered heterocycle; wherein the 3 to 8-membered heterocycle is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, and —NO₂;
R³ is selected from optionally substituted C₁-C₆ alkyl, optionally substituted 3- to 10-membered heterocycle and optionally substituted C₃₋₁₀ carbocycle, wherein each of the alkyl, heterocycle and carbocycle is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
R⁴ is selected from:
hydrogen;
optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
Ring W is an optionally substituted 5 or 6-membered heteroaryl, wherein the 5 or 6-membered heteroaryl is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle,
wherein the method comprises reacting a compound represented by Formula (E), or a pharmaceutically acceptable salt thereof, 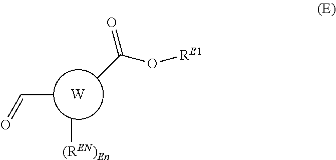
wherein,
Ring W is defined above,
En is 0,
R^{EN} isa protecting group, and
R^{E1} is C₁-C₆ alkyl (e.g., ethyl), C₁-C₆haloalkyl, C₁-C₆hydroxyalkyl, C₁-C₆aminoalkyl, C₁-C₆heteroalkyl, substituted or unsubstituted aryl, or substituted or unsubstituted heteroaryl;
with a compound represented by Formula (F), or a pharmaceutically acceptable salt thereof, 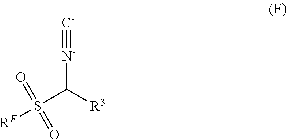
wherein R³ is defined above, and
R^{F} is a substituted or unsubstituted phenyl,
thereby producing a compound of Formula (G-a), or a pharmaceutically acceptable salt thereof, 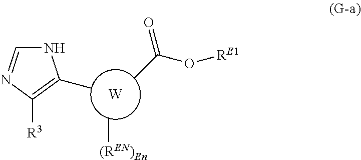
wherein R³, Ring W, En, R^{EN} and are defined above,
and wherein the method further comprises reacting the compound of Formula (G-a), or a pharmaceutically acceptable salt thereof, with a compound of Formula (J), or a pharmaceutically acceptable salt thereof,
R⁴—R^{J} (J)
wherein R is a leaving group,
thereby producing a compound represented by Formula (G), or a pharmaceutically acceptable salt thereof, 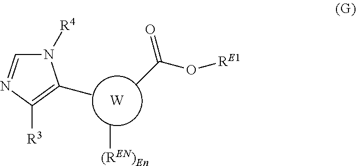
wherein R³, R⁴, R^{EN} and R^{E} are defined above.

47. The method of claim 46, wherein the compound of Formula (E) has a structure of

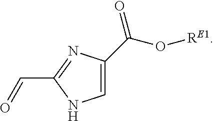

48. The method of claim 46 or 47, wherein the compound of Formula (G-a) has a structure of

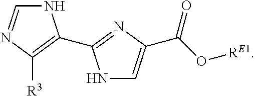

49. The method of claim 46, wherein the compound of Formula (G) has a structure of

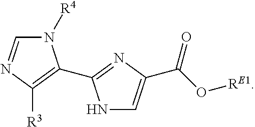

50. The method of any one of claims 46 to 49, wherein R^{J} is a halogen (e.g., Br, Cl, I).

51. The method of any one of claims 46 to 49, wherein R^{J} is alkylsulfonate (e.g., trifluoromethanesulfonate (triflate or TfO⁻)) or arylsulfonate (e.g., 4-methylbenzenesulfonate).

52. The method of any one of claims 46 to 51, wherein the method further comprises a deprotecting reaction.

53. The method of claim 52, wherein the deprotecting reaction occurs before the reacting of the compound represented by Formula (E) with the compound represented by Formula (F).

54. The method of claim 53, wherein the compound represented by Formula (E) is deprotected.

55. The method of any one of claims 46 to 54, further comprising subjecting the compound represented by Formula (G) or a pharmaceutically acceptable salt thereof, to a hydrolysis rection, thereby producing a compound of Formula (H), or a pharmaceutically acceptable salt thereof, 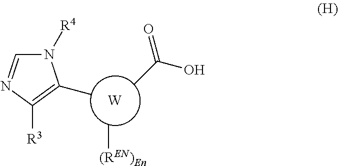
wherein R³, R⁴, En and R^{EN} are defined above.

56. The method of claim 55, wherein the compound of Formula (H) has a structure of

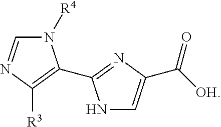

57. The method of claim 55 or 56, further comprising subjecting the compound represented by Formula (H) or a pharmaceutically acceptable salt thereof, through a condensation reaction with a compound of Formula (C), or a pharmaceutically acceptable salt thereof, 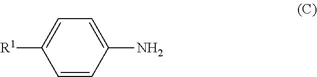
wherein R¹ is defined above in Formula (Ia).

58. The method of any one of claims 46 to 57, wherein the compound represented by Formula (I) has a structure of Formula (Ia), 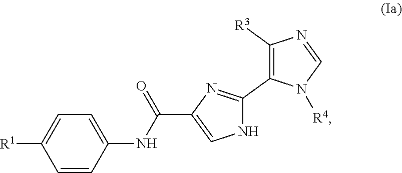
wherein:
R¹ is piperazine, wherein the piperazine is optionally substituted with one or more substituents independently selected at each occurrence from oxo and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is substituted C₁-C₆ alkyl, wherein the C₁-C₆ alkyl is substituted with one or more halogen.

59. The method of any one of claims 46 to 57, wherein the compound represented by Formula (I) has a structure of Formula (Ia), 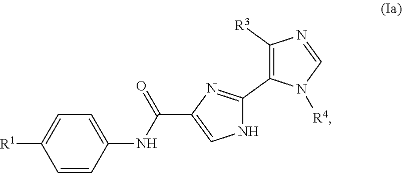
wherein,
R¹ is piperazine, wherein the piperazine is optionally substituted with one or more substituents independently selected at each occurrence from oxo, —S(O₂)NH₂, and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is unsubstituted C₁-C₆ alkyl.

60. A method of producing a compound represented by Formula (Ia), or a pharmaceutically acceptable salt thereof, 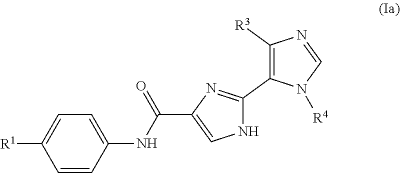
wherein:
R¹ is piperazine, wherein the piperazine is optionally substituted with one or more substituents independently selected at each occurrence from oxo, —S(O₂)NH₂, and C₁₋₁₀ alkyl, wherein the C₁₋₁₀ alkyl is optionally substituted with one or more substituents independently selected at each occurrence from hydroxy, halogen, oxo, and —NH₂;
R³ is phenyl, wherein the phenyl is optionally substituted with one or more halogen; and
R⁴ is unsubstituted C₁-C₆ alkyl;
wherein the method comprises reacting a compound represented by Formula (E-1), or a pharmaceutically acceptable salt thereof, 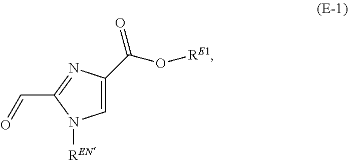
wherein,
R^{EN′} is hydrogen or R^{EN}, and wherein R^{EN} is a protecting group;
R^{E1} is C₁-C₆ alkyl (e.g., ethyl), C₁-C₆haloalkyl, C₁-C₆hydroxyalkyl, C₁-C₆aminoalkyl, C₁-C₆heteroalkyl, substituted or unsubstituted aryl, or substituted or unsubstituted heteroaryl;
with a compound represented by Formula (F), or a pharmaceutically acceptable salt thereof, 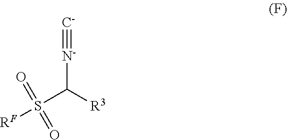
wherein R³ is defined above, and
R^{F} is a substituted or unsubstituted phenyl,
thereby producing a compound of Formula (G-1a), or a pharmaceutically acceptable salt thereof, 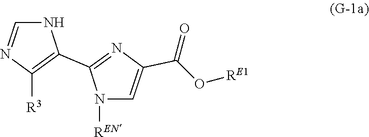
wherein R³, R^{EN′} and R^{E1} are defined above,
and wherein the method further comprises reacting the compound of Formula (G-1a), or a pharmaceutically acceptable salt thereof, with a compound of Formula (J), or a pharmaceutically acceptable salt thereof,
R⁴—R (J)
wherein R is a leaving group,
thereby producing a compound represented by Formula (G-1), or a pharmaceutically acceptable salt thereof, 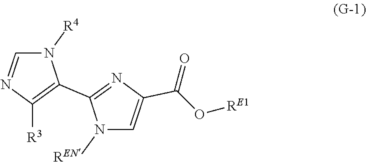
wherein R³, R⁴, R^{EN′} and R^{E1} are defined above.

61. The method of claim 60, further comprising subjecting the compound represented by Formula (G-1) or a pharmaceutically acceptable salt thereof, to a hydrolysis rection, thereby producing a compound of Formula (H-1), or a pharmaceutically acceptable salt thereof, 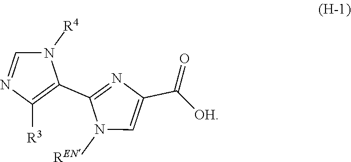
wherein R³, R⁴, and R^{EN′} are defined above.

62. The method of claim 61, further comprising subjecting the compound represented by Formula (H-1) or a pharmaceutically acceptable salt thereof, through a condensation reaction with a compound of Formula (C), or a pharmaceutically acceptable salt thereof, 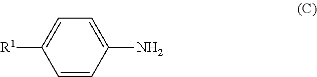
wherein R¹ is defined above in Formula (Ia).

63. The method of any one of claim 60 to 62, wherein R^{EN′} is hydrogen.

64. The method of any one of claim 60 to 62, wherein R^{EN′} is R^{EN}.

65. The method of claim 64, wherein R^{EN} is selected from (trimethyl silicon)ethoxymethyl (SEM), methyloxycarbonyl, ethyloxycarbonyl, benzyloxycarbonyl (CBz), tert-butoxycarbonyl (Boc), 9-fluorenylmethyloxycarbonyl (Fmoc), allyloxycarbonyl (Alloc), 2-(trimethylsilyl)ethyloxycarbonyl (Teoc), 2,2,2-trichloroethoxycarbonyl (Troc), para-toluenesulfonyl (Tos), 2,2,2-trifluoroacetyl (Tfa), trityl (Trt), 2,4-dimethocybenzyl (Dmb), p-Methoxybenzyl (Pmb), and benzyl (Bn).

66. The method of claim 64, wherein R^{EN} is SEM.

67. The method of any one of claims 60 to 65, wherein the method further comprises a deprotecting reaction.

68. The method of claim 67, wherein the deprotecting reaction occurs after the condensation reaction, and wherein the deprotecting reaction comprises replacing R^{EN} with a hydrogen.

69. The method of claim 67, wherein the deprotecting reaction occurs before the reacting of the compound represented by Formula (E-1) with the compound represented by Formula (F).

70. The method of claim 69, wherein the compound represented by Formula (E-1) is deprotected.

71. The method of any one of claims 16, 40, 57 or 62, wherein the compound of Formula (C), or a pharmaceutically acceptable salt thereof, has a structure of

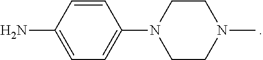

72. A compound represented by Formula (A), or a pharmaceutically acceptable salt thereof, 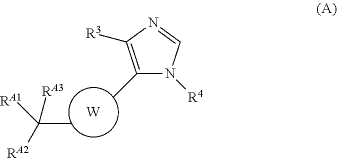
wherein
R³ is selected from optionally substituted C₁-C₆ alkyl, optionally substituted 3- to 10-membered heterocycle and optionally substituted C₃₋₁₀ carbocycle, wherein each of the alkyl, heterocycle and carbocycle is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R⁴ is selected from:
hydrogen;
optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
Ring W is an optionally substituted 5 or 6-membered heteroaryl, wherein the 5 or 6-membered heteroaryl is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
each of R^{A1}, R^{A2} and R^{A3} is independently selected from a halogen and —O—C₁₋₆alkyl.

73. The compound of claim 72, wherein the compound has a structure of [chemical structure]
